(19) United States
(12) Patent Application Publication
(10) Pub. No.: US 2005/0123538 A1
(43) Pub. Date: Jun. 9, 2005

(54) Polynucleotides encoding novel ErbB-2 polypeptides and kits and methods using same

(76) Inventors: Ronen Shemesh, ModiIn (IL); Anat Oren, Tel Aviv (IL); Galit Rotman, Herzlia (IL); Osnat Sella-Tavor, Gat-Rimon (IL); Shira Walach, Hod-HaSharon (IL); Shirley Sameach-Greenwald, Kfar-Saba (IL); Merav Beiman, Rehovot (IL); Dani Eshel, Karkur (IL); Kinneret Savitsky, Tel Aviv (IL)

Correspondence Address: ANTHONY CASTORINA, SUITE 207, 2001 JEFFERSON DAVIS HIGHWAY, ARLINGTON, VA, 22202, US

(21) Appl. No.: 10/956,373

(22) Filed: Oct. 4, 2004

Related U.S. Application Data
(60) Provisional application No. 60/507,953, filed on Oct. 3, 2003.
(60) Provisional application No. 60/518,321, filed on Nov. 10, 2003.
(60) Provisional application No. 60/599,583, filed on Aug. 9, 2004.
(60) Provisional application No. 60/607,326, filed on Sep. 7, 2004.

(51) Int. Cl. C12Q 1/68; C07H 21/04; A61K 39/395; C07K 14/72; C07K 16/28

(52) U.S. Cl. 424/143.1; 435/6; 435/69.1; 435/320.1; 435/325; 530/350; 530/388.22; 536/23.5

(57) ABSTRACT

Isolated polynucleotides encoding novel ErbB-2 polypeptides are provided. Also provided are methods and kits using same for diagnosing prognosing and treating ErbB-related cancer.

The teachings of U.S. Provisional Applications No. 60/507,953 filed Oct. 3, 2003, 60/518,321 filed Nov. 10, 2003, 60/599,583 filed Aug. 9, 2004 and 60/607,326 filed Sep. 7, 2004 are hereby incorporated by reference.

### FIELD OF THE INVENTION

The present invention relates to novel ErbB-2 polypeptides and polynucleotides encoding thereof, more particularly, to methods and kits using same for diagnosing and treating ErbB-2-related pathology, such as breast cancer for example.

### BACKGROUND OF THE INVENTION

Human epidermal growth factor receptor-2 (ErbB-2/HER-2/neu) is a protooncogene encoding cell-surface glycoprotein receptor-like tyrosine kinase (RTK) which plays a central role in mammalian embryogenesis (Lee et al., Nature 378:394-398, 1995) and in the development of several human carcinomas [Hynes and Stem, Biochim. et Biophy. Acta 1198:165-184, (1994); and Dougall et al., Oncogene 9:2109-2123, (1994)].

The protein sequence of ErbB-2 was initially determined from a cDNA which was cloned by homology to the epidermal growth factor receptor (EGFR) mRNA from placenta [Coussens et al., Science 230:1132-1139, (1985)] and from a gastric carcinoma cell line [Yamamoto et al., Nature 319:230-234, (1986)].

The ErbB-2 mRNA is about 45 kb [Coussens et al. (1985) supra; and Yamamoto et al., (1986) supra] encoding a transmembrane glycoprotein of 185 kDa which can be expressed in both normal and malignant human tissues [p185HER-2, Hynes and Steen, Biochim. et Biophys. Acta 1198:165-184, (1994); and Dougall et al., Oncogene 9:2109-2123, (1994)].

The function of the ErbB-2 gene has been elucidated mainly by ectopic expression of the 4.5 kb transcript in cell-lines and subsequent analysis of the structure and biochemical properties of the 185 kDa protein product.

Thus, the ErbB-2 protein consists of a large extracellular domain, a transmembrane segment, and an intracellular domain exhibiting tyrosine kinase activity [Hynes and Stern, Biochim. et Biophys. Acta 1198:165-184, (1994); and Dougall et al., Oncogene 9:2109-2123, (1994)]. Overexpression of ErbB-2 causes phenotypic transformation of cultured cells [DiFiore et al., Science 237:178-182, (1987); and Hudziak et al., Proc. Natl. Acad. Sci. USA 84:7159-7163, (1987)] and has been associated with aggressive clinical progression of breast and ovarian cancer [Slamon et al., Science 235:177-182, (1987); and Slamon et al., Science 244:707-712, (1989)].

ErbB-2 is highly homologous to the EGFR and other members of the ErbB family, ErbB-3 and ErbB-4. To date, no direct ligand to ErbB-2 has been identified. Moreover, the signaling activity of ErbB-2 is mediated by heterodimerization with other ligand-binding members of the EGFR family [Carraway and Cantley, Cell 78:5-8, (1994); Earp et al., Breast Cancer Res. Treat. 35:115-132, (1995); and Qian et al., Oncogene 10:211-219, (1995)], which activate the ErbB-2 protooncogene in the absence of a direct ligand.

Since ErbB-2 gene amplification and receptor overexpression by tumors is associated with patient's poor prognosis and may be predictive of response to certain anticancer therapies, a wide range of techniques are used for the detection of ErbB-2 status.

Two predominant technologies are routinely practiced in clinical pathology laboratories; determination of ErbB-2 protein overexpression by immunohistochemistry (IHC) and ErbB-2 gene amplification by fluorescence in-situ hybridization (FISH), which when used together detect ErbB-2 status at a relatively high level of success [80-90%, Di Leo (2002) Oncology 63 (suppl. 1):25-32].

However, accurate diagnostic assessment of patients, is a prerequisite for the appropriate usage of cancer therapy (e.g., Herceptin) for the treatment of HER-2 overexpressing metastatic breast cancer: false-negative results may deny patients the chance of life extending therapy, while false-positive results waste resources, give rise to false hopes and expose patients to unnecessary adverse effects.

A number of clinical findings rendered ErbB-2 an attractive target for cancer therapy: The level of HER2 expression found in human cancer cells where gene amplification occurs is much higher than that found in normal adult tissues. A second attractive aspect of the HER2 target is that it is present in a very high proportion of tumor cells [Press Oncogene (1990) 5:953-962], and tumors with high expression (score 3+) show uniform intense immunohistochemical staining [Esteva Breast Cancer Res Treat (1999);57:17a]. This characteristic suggests that, in a given patient, anti-HER2 therapy should be able to attack nearly all cancer cells. Finally, the HER2 overexpression phenotype is apparently shared between the primary tumor and metastatic sites [Niehans J Natl Cancer Inst (1993) 85:1230-1235] suggesting that therapy for metastatic disease can be selected based on analysis of the primary tumor, and again indicates that an anti-HER2 therapy should be able to treat all sites of disease. The strong linkage to the pathogenesis of breast cancer and its association with prognosis made HER2 a target for the development of new cancer therapies [Esteva Ref Gynecol Obst 2000;7:267-276; Pusztai Cancer Treat Rev 1999;25:271-277]. Such therapies include monoclonal antibodies which are directed against the ErbB-2 thereby reducing the growth rate of human tumor cells and sensitizing cancer cells to chemotherapeutic agents, ErbB-2 directed vaccines and kinase inhibitors.

ErbB splice variants that encode truncated ECDs have been suggested to modulate ErbB signaling [Maihle (2002) Cancer Treatment Res. 107, 247-258] either by sequestering growth factors [Lee (2001) Cancer Res. 61, 4467-4473] or by altering receptor interactions. One of these, herstatin, is a secreted alternative product of the HER-2 gene containing ECD subdomains I and II followed by an intron-encoded 79-amino acid sequence [Doherty (1999) Proc. Natl. Acad. Sci. U.S.A. 96, 10869-10874]. Herstatin has been shown to bind to EGFR and HER-2 and to block homomeric and heteromeric receptor interactions [Azios (2001) Oncogene 20, 5199-5209L; Doherty (1999) Supra]. In contrast to dominant negative mutants, herstatin does not require a membrane anchor to achieve complex formation and trans inhibition, suggesting that its novel C-terminal domain may confer high affinity binding to the receptors. Indeed, the intron-encoded domain, expressed as a recombinant peptide, binds to HER-2 and the EGFR [Azios (2001) supra; Doherty (1999) supra].

While reducing the present invention to practice the present inventors uncovered novel naturally occurring modulators of ErbB-2 signaling, which can be used to diagnose and treat ErbB-related cancer.

### SUMMARY OF THE INVENTION

According to one aspect of the present invention there is provided an isolated polynucleotide comprising a nucleic acid sequence encoding at least an active portion of an ErbB-2 polypeptide including an amino acid sequence being at least 70% homologous to SEQ ID NO:5, as determined using the BlastP software of the National Center of Biotechnology Information (NCBI) using default parameters.

According to another aspect of the present invention there is provided an isolated polynucleotide comprising a nucleic acid sequence encoding at least an active portion of an ErbB-2 polypeptide including an amino acid sequence being at least 70% homologous to SEQ ID NO:6, as determined using the BlastP software of the National Center of Biotechnology Information (NCBI) using default parameters, optionally and preferably including the following: filtering on (this option filters repetitive or low-complexity sequences from the query using the SEG (protein) program), scoring matrix is BLOSUM62 for proteins, word size is 3, E value is 10, gap costs are 11, 1 (initialization and extension), and number of alignments shown is 50.

It should be noted that homology for nucleic acid sequences described herein is preferably determined using the BlastN software of the National Center of Biotechnology Information (NCBI) using default parameters, including using the DUST filter program.

According to further features in preferred embodiments of the invention described below, a nucleic acid construct comprises the isolated polynucleotide.

According to still further features in the described preferred embodiments the nucleic acid construct further comprises a promoter for regulating transcription of the isolated polynucleotide in sense or antisense orientation.

According to still further features in the described preferred embodiments the nucleic acid construct further comprises a positive and a negative selection markers for selecting for homologous recombination events.

According to still further features in the described preferred embodiments a host cell comprises the nucleic acid construct.

According to yet another aspect of the present invention there is provided an isolated polypeptide as set forth in SEQ ID NO:2, 4 or 10 or an active portion thereof.

According to still another aspect of the present invention there is provided an isolated polypeptide comprising an ErbB-2 polypeptide including an amino acid sequence being at least 70% homologous to SEQ ID NO:6, as determined using the BlastP software of the National Center of Biotechnology Information (NCBI) using default parameters.

According to an additional aspect of the present invention there is provided an antibody or an antibody fragment being capable of specifically binding an ErbB-2 polypeptide including an amino acid sequence being at least 70% homologous to SEQ ID NO:5, as determined using the BlastP software of the National Center of Biotechnology Information (NCBI) using default parameters.

According to yet an additional aspect of the present invention there is provided an antibody or an antibody fragment being capable of specifically binding an ErbB-2 polypeptide including an amino acid sequence being at least 70% homologous to SEQ ID NO:6, as determined using the BlastP software of the National Center of Biotechnology Information (NCBI) using default parameters.

According to still an additional aspect of the present invention there is provided a display library comprising a plurality of display vehicles each displaying at least 6 consecutive amino acids derived from an ErbB-2 polypeptide including an amino acid sequence being at least 70% homologous to SEQ ID NO:5 as determined using the BlastP software of the National Center of Biotechnology Information (NCBI) using default parameters.

According to a further aspect of the present invention there is provided a display library comprising a plurality of display vehicles each displaying at least 6 consecutive amino acids derived from an ErbB-2 polypeptide including an amino acid sequence being at least 70% homologous to SEQ ID NO:6 as determined using the BlastP software of the National Center of Biotechnology Information (NCBI) using default parameters.

According to yet a further aspect of the present invention there is provided an oligonucleotide specifically hybridizable with a nucleic acid sequence encoding an ErbB-2 polypeptide including an amino acid sequence being at least 70% homologous to SEQ ID NO:5, as determined using the BlastP software of the National Center of Biotechnology Information (NCBI) using default parameters.

According to still further features in the described preferred embodiments the nucleic acid sequence is as set forth in SEQ ID NO:1, 7 or 13.

According to still a further aspect of the present invention there is provided an oligonucleotide specifically hybridizable with a nucleic acid sequence encoding an ErbB-2 polypeptide including an amino acid being at least 70% homologous to SEQ ID NO:6, as determined using the BlastP software of the National Center of Biotechnology Information (NCBI) using default parameters.

According to still further features in the described preferred embodiments the nucleic acid sequence is as set forth in SEQ ID NO:3, 8 or 15.

According to still further features in the described preferred embodiments the oligonucleotide is a single or double stranded.

According to still further features in the described preferred embodiments the oligonucleotide is at least 10 bases long.

According to still further features in the described preferred embodiments the oligonucleotide is hybridizable in either sense or antisense orientation.

According to still a further aspect of the present invention there is provided a pharmaceutical composition comprising a therapeutically effective amount of at least an active portion of an ErbB-2 polypeptide including an amino acid sequence being at least 70% homologous to SEQ ID NO:5, as determined using the BlastP software of the National Center of Biotechnology Information (NCBI) using default parameters.

According to still further features in the described preferred embodiments the polypeptide is as set forth in SEQ ID NO:2, 5 or 14.

According to still further features in the described preferred embodiments the active portion of the polypeptide is as set forth in SEQ ID NO:5.

According to still further features in the described preferred embodiments the active portion of the polypeptide is encoded by nucleotide coordinates 2097-2320 of SEQ ID NO:1.

According to still a further aspect of the present invention there is provided a pharmaceutical composition comprising a therapeutically effective amount of at least an active portion of an ErbB-2 polypeptide including an amino acid sequence being at least 70% homologous to SEQ ID NO:6, as determined using the BlastP software of the National Center of Biotechnology Information (NCBI) using default parameters.

According to still further features in the described preferred embodiments the polypeptide is as set forth in SEQ ID NO:4, 6 or 16.

According to still further features in the described preferred embodiments the active portion of the polypeptide is as set forth in SEQ ID NO:6.

According to still further features in the described preferred embodiments in the active portion of the polypeptide is encoded by nucleotide coordinates 1664-1944 of SEQ ID NO:3.

According to still a further aspect of the present invention there is provided a method of diagnosing predisposition to, or presence of ErbB-related cancer in a subject, the method comprising determining a level of a polypeptide at least 70% homologous to SEQ ID NO:5, as determined using the Blastn software of the National Center of Biotechnology information (NCBI) using default parameters or of a polynucleotide encoding the polypeptide in a biological sample obtained from the subject, wherein the level of the polynucleotide or the level of the polypeptide level is correlatable with predisposition to, or presence or absence of the cancer, thereby diagnosing predisposition to, or presence of ErbB-related cancer in the subject. ErbB-related cancer is any cancer where ErbB-2 encoded mRNAs and/or polypeptides are differentially expressed as compared to a non-cancerous condition.

According to still further features in the described preferred embodiments the polynucleotide is selected from the group consisting of SEQ ID NOs: 1, 7 and 13.

According to still further features in the described preferred embodiments the polypeptide is selected from the group consisting of SEQ ID NOs: 2, 5 and 14.

According to still a further aspect of the present invention there is provided a method of diagnosing predisposition to, or presence of ErbB-related cancer in a subject, the method comprising determining a level of a polypeptide at least 70% homologous to SEQ ID NO:6, as determined using the Blastn software of the National Center of Biotechnology information (NCBI) using default parameters or of a polynucleotide encoding the polypeptide in a biological sample obtained from the subject, wherein the level of the polynucleotide or the level of the polypeptide level is correlatable with predisposition to, or presence or absence of the cancer, thereby diagnosing predisposition to, or presence of ErbB-related cancer in the subject.

According to still further features in the described preferred embodiments the determining level of the polypeptide is effected via an assay selected from the group consisting of immunohistochemistry, ELISA, RIA, Western blot analysis, FACS analysis, an immunofluorescence assay, and a light emission immunoassay.

According to still further features in the described preferred embodiments the determining level of the polynucleotide is effected via an assay selected from the group consisting of PCR, RT-PCR, chip hybridization, RNase protection, in-situ hybridization, primer extension, Southern blot, Northern blot and dot blot analysis.

According to still further features in the described preferred embodiments the polynucleotide is selected from the group consisting of SEQ ID NOs: 3, 8 and 15.

According to still further features in the described preferred embodiments the polypeptide is selected from the group consisting of SEQ ID NOs: 4, 6 and 16.

According to still a further aspect of the present invention there is provided a method of treating ErbB-related cancer in a subject, the method comprising specifically upregulating in the subject expression of an ErbB-2 polypeptide including an amino acid sequence being at least 70% homologous to SEQ ID NO:5 as determined using the Blastp software of the National Center of Biotechnology information (NCBI) using default parameters.

According to still a further aspect of the present invention there is provided an isolated polynucleotide comprising a nucleic acid sequence encoding an ErbB-2 polypeptide including an amino acid sequence at least 70% homologous to SEQ ID NO:28, as determined using the protein Blast search software for short, nearly exact matches of the National Center of Biotechnology Information (NCBI) using default parameters.

According to still further features in the described preferred embodiments the nucleic acid sequence is as set forth in SEQ ID NO:25.

According to still a further aspect of the present invention there is provided an isolated polynucleotide as set forth in SEQ ID NO:25 or an active portion thereof.

According to still a further aspect of the present invention there is provided an isolated polynucleotide encoding at least an active portion of an ErbB-2 polypeptide including an inositol phosphate binding domain.

According to still further features in the described preferred embodiments the active portion is encoded by nucleic acid sequence coordinates 1171-1314 of SEQ ID NO:25.

According to still further features in the described preferred embodiments the inositol phosphate binding domain is encoded by nucleic acid sequence coordinates 1171-1314 of SEQ ID NO:25.

According to still further features in the described preferred embodiments the inositol phosphate binding domain is a pleckstrin homology domain.

According to still a further aspect of the present invention there is provided an isolated polypeptide comprising an ErbB-2 polypeptide including an amino acid sequence being at least 70% homologous to SEQ ID NO:28, as determined using the protein Blast search software for short, nearly exact matches of the National Center of Biotechnology Information (NCBI) using default parameters.

According to still further features in the described preferred embodiments the ErbB-2 polypeptide is as set forth in SEQ ID NO:26.

According to still a further aspect of the present invention there is provided a nucleic acid construct comprising the isolated polypeptide.

According to still a further aspect of the present invention there is provided a host cell comprising the nucleic acid construct.

According to still a further aspect of the present invention there is provided an antibody or an antibody fragment being capable of specifically binding a polypeptide sequence at least 70% homologous to SEQ ID NO:28, as determined using the protein Blast search software for short, nearly exact matches of the National Center of Biotechnology Information (NCBI) using default parameters.

According to still further features in the described preferred embodiments the ErbB-2 polypeptide is as set forth in SEQ ID NO:26.

According to still a further aspect of the present invention there is provided an oligonucleotide specifically hybridizable with a nucleic acid sequence encoding a polypeptide at least 70% homologous to SEQ ID NO:28, as determined using the protein Blast search software for short, nearly exact matches of the National Center of Biotechnology Information (NCBI) using default parameters.

According to still further features in the described preferred embodiments the oligonucleotide is as set forth in SEQ ID NO:31 or 32.

According to still further features in the described preferred embodiments the oligonucleotide is a single or double stranded.

According to still further features in the described preferred embodiments the oligonucleotide is at least 10 bases long.

According to still further features in the described preferred embodiments the oligonucleotide is hybridizable in either sense or antisense orientation.

According to still a further aspect of the present invention there is provided a method of diagnosing predisposition to, or presence of ErbB-related cancer in a subject, the method comprising determining a level of a polypeptide at least 70% homologous to SEQ ID NO:28, as determined using the protein Blast search software for short, nearly exact matches of the National Center of Biotechnology Information (NCBI) using default parameters or of a polynucleotide encoding the polypeptide in a biological sample obtained from the subject, wherein the level of the polynucleotide or the level of the polypeptide level is correlatable with predisposition to, or presence or absence of the cancer, thereby diagnosing predisposition to, or presence of ErbB-related cancer in the subject.

According to still further features in the described preferred embodiments the upregulating expression of the polypeptide is effected by:
(i) administering the polypeptide to the subject; and/or
(ii) administering an expressible polynucleotide encoding the polypeptide to the subject.

According to still further features in the described preferred embodiments specifically downregulating expression of the polypeptide is effected by providing to the subject an antibody directed at an amino acid sequence set forth in SEQ ID NO:5.

According to still further features in the described preferred embodiments the oligonucleotide is directed at a nucleic acid sequence set forth in SEQ ID NO:7.

According to still a further aspect of the present invention there is provided a method of treating ErbB-related cancer in a subject, the method comprising specifically upregulating in the subject expression of an ErbB-2 polypeptide including an amino acid sequence being at least 70% homologous to SEQ ID NO:6, as determined using the Blastp software of the National Center of Biotechnology information (NCBI) using default parameters.

According to still further features in the described preferred embodiments specifically downregulating expression of the polypeptide is effected by providing to the subject an antibody directed at an amino acid sequence set forth in SEQ ID NO:6.

According to still further features in the described preferred embodiments specifically downregulating expression of the polypeptide is effected by providing to the subject an oligonucleotide capable of specifically inactivating the polynucleotide.

According to still further features in the described preferred embodiments the oligonucleotide is directed at a nucleic acid sequence set forth in SEQ ID NO:8.

According to still further features in the described preferred embodiments the ErbB-2 long (for example variants I, IV) or short (for example variants II, V) variants of the present invention, detected by amplicons as depicted in SEQ ID NOs: 50 (variant I) or 53 (variant II), respectively, are differentially expressed in breast cancer as compared to normal breast tissue, such that preferably a higher level of expression is observed with the splice variants of the present invention in breast cancer tissue than in normal breast tissue. According to still a further aspect of the present invention there is provided novel markers for breast cancer that are both sensitive and accurate. The measurement of these markers, alone or in combination, in patient samples provides information that the diagnostician can correlate with a probable diagnosis of breast cancer. The markers of the present invention, alone or in combination show a high degree of differential detection between breast cancer and non-cancerous states.

The present invention therefore also relates to diagnostic assays for breast cancer, and methods of use of such markers for detection of breast cancer (alone or in combination) in a sample taken from a subject (patient). The assays are preferably NAT (nucleic acid amplification technology)-based assays, such as PCR for example (or variations thereof such as real-time PCR for example), but may optionally also feature detection of a protein and/or peptide, for example by using an antibody for such detection. Non-limiting examples of immunoassays encompassed by the present invention include a Western blot assay or an ELISA, although of course other immunoassays could optionally be used. In the case of an immunoassay, the present invention also comprises antibodies to at least a portion of a splice variant of the present invention, preferably such that the antibody binds at least preferentially (and more preferably exclusively) to a splice variant of ErbB-2 according to the present invention over wild type ErbB-2. Indeed, most preferably such antibodies cannot specifically bind to wild type ErbB-2, but only to a splice variant of ErbB-2 according to the present invention. The present invention also optionally encompasses immunocomplexes, comprising such an antibody specifically bound to an epitope on a splice variant of ErbB-2 according to the present invention, wherein the epitope is preferably not present in wild type ErbB-2. The assays may also optionally encompass nucleic acid hybridization assays. The assays may optionally be qualitative or quantitative.

The present invention also relates to kits based upon such diagnostic methods or assays.

In certain embodiments, the sample taken from the subject can be selected from one or more of seminal plasma, blood, serum, urine, or any other bodily fluid or secretion or tissue sample.

According to still a further aspect of the present invention there is provided a kit for diagnosing ErbB-related cancer or a predisposition thereto in a subject, the kit comprising the antibody or antibody fragment of the present invention and reagents for detecting hybridization of the antibody or antibody fragment.

According to still further features in the described preferred embodiments detecting hybridization of the antibody or antibody fragment is effected by an assay selected from the group consisting of immunohistochemistry, ELISA, RIA, Western blot analysis, FACS analysis, an immunofluorescence assay, and a light emission immunoassay.

According to still further features in the described preferred embodiments the antibody or antibody fragment is coupled to an enzyme.

According to still further features in the described preferred embodiments the antibody or antibody fragment is coupled to a detectable moiety selected from the group consisting of a chromogenic moiety, a fluorogenic moiety, a radioactive moiety and a light-emitting moiety.

According to still a further aspect of the present invention there is provided a kit for diagnosing ErbB-related cancer or a predisposition thereto in a subject, the kit comprising the oligonucleotide of the present invention and at least one reagent for detecting hybridization of the oligonucleotide.

According to still further features in the described preferred embodiments the at least one reagent is selected suitable for detecting hybridization via an assay selected from the group consisting of PCR, RT-PCR, chip hybridization, RNase protection, in-situ hybridization, primer extension, Southern blot, Northern blot and dot blot analysis.

According to still a further aspect of the present invention there is provided an isolated polynucleotide comprising a nucleic acid sequence encoding at least an active portion of an ErbB-2 polypeptide including an amino acid sequence being at least 90% homologous to SEQ ID NO:11, as determined using the BlastP software of the National Center of Biotechnology Information (NCBI) using default parameters.

According to still a further aspect of the present invention there is provided an isolated polypeptide comprising an ErbB-2 polypeptide including an amino acid sequence being at least 90% homologous to SEQ ID NO:11, as determined using the BlastP software of the National Center of Biotechnology information (NCBI) using default parameters or an active portion thereof.

According to still further features in the described preferred embodiments the amino acid sequence is as set forth in SEQ ID NO:10, 11, 14 or 16.

According to still further features in the described preferred embodiments the nucleic acid sequence is as set forth in SEQ ID NO. 9, 12, 13 or 15.

According to still further features in the described preferred embodiments the active portion of the polypeptide is as set forth is SEQ ID NO:11.

According to still a further aspect of the present invention there is provided a biomarker for detecting breast cancer, comprising ErbB-2 long variants (such as ErbB-2 variants I or IV) sequence or a fragment thereof.

According to still a further aspect of the present invention there is provided the biomarker as above, wherein the fragment comprises a tail of ErbB-2-long variant (such as ErbB-2 variants I or IV), comprising a polypeptide having the sequence RLAWTPGCTLHCPSLPHWMLGGHCCREGTP (SEQ ID NO: 5) or a polynucleotide encoding the polypeptide.

According to still a further aspect of the present invention there is provided a biomarker for detecting breast cancer, comprising ErbB-2-short variant (such as ErbB-2 variants II or V) sequence or a fragment thereof.

According to still a further aspect of the present invention there is provided the biomarker as above, wherein the fragment comprises a tail of ErbB-2-short variant (such as ErbB-2 variants II or V), comprising a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6) or a polynucleotide encoding the polypeptide.

It should be noted that the terms “ErbB-2 variant I”, “VarI”, “long variant”, “Cgen-B2L” and “B2L” are used interchangeably, and all refer to the long variant of ErbB-2, as shown with regard to SEQ ID NO: 2. Also, the terms “ErbB-2 variant II”, “short variant”, “VarI”, “Cgen-B2S” and “B2S” are used interchangeably, and all refer to the short variant of ErbB-2, as shown with regard to SEQ ID NO: 4.

Also, generally terms such as “variant 1”, “VarI”, or “variant I” are used interchangeably.

According to still a further aspect of the present invention there is provided a primer pair for use in detecting the biomarkers, comprising a primer pair capable of amplifying ErbB-2-long variants (such as ErbB-2 variants I or IV), ErbB-2-short variants (such as ErbB-2 variants II or V) or a fragment thereof.

According to still a further aspect of the present invention is the the primer pair comprising ErbB-2-long variant-forward primer: TGTGAGGGACACAGGCAAAGT (SEQ ID NO: 48); and ErbB-2-long variant—Reverse primer: CCCACCATCCCCAGTTAAGAA (SEQ ID NO: 49).

According to still a further aspect of the present invention, there is provided a primer pair, comprising ErbB-2-short variant—forward primer: CAGCGTTCTTGGACTTGTGC (SEQ ID NO: 51); and ErbB-2-short variant—Reverse primer: CCAGCTAGAGAAGCCATGCC (SEQ ID NO: 52).

According to still a further aspect of the present invention, there is provided an amplicon obtained through the use of the primer pairs.

According to still a further aspect of the present invention, there is provided an amplicon, comprising ErbB-2-long variant amplicon:

(SEQ ID NO 50)

TGTGAGGGACACAGGCAAAGTTCAATTCCTTGGAAGTCAAGGGAGACTGA
GAAGAGTACAGCTGCAGCACTGAGGGAGTGATGAATTCTTAACTGGGGAT
GGTGGG.

According to still a further aspect of the present invention, there is provided the amplicon, comprising ErbB-2-short variant amplicon:

| | |
|---|---|
| CAGCGTTCTTGGACTTGTGCAGACTGCCCGTCTCTGTGCACCCTTCTTGAC | (SEQ ID NO 53) |
| TCAGCACAGCTCTGGCTGGCTTGGCCTCTTGGCATGGCTTCTCTAGCTGG. | |

According to still a further aspect of the present invention, there is provided an assay for detecting breast cancer, comprising: an assay detecting overexpression of ErbB-2-long or ErbB-2-short variants (such as ErbB-2 variants I, IV or II, V, respectively) or a fragment thereof.

According to still a further aspect of the present invention the assay comprises a NAT-based technology.

According to still a further aspect of the present invention, there is provided a method for detecting breast cancer, comprising: detecting overexpression of the ErbB-2 variants or a fragment thereof.

According to the present invention, there is provided a method of diagnosing predisposition to, or presence, or prognosis, or monitoring the progression of, or a responsiveness to treatment, of ErbB-related cancer in a subject, the method comprising determining a level of a polypeptide at least 71% homologous to SEQ ID NO:5, as determined using the Blastn software of the National Center of Biotechnology information (NCBI) using default parameters or of a polynucleotide encoding said polypeptide in a biological sample obtained from the subject, wherein said level of said polynucleotide or said level of said polypeptide level is correlatable with predisposition to, or presence or absence of the cancer, thereby diagnosing predisposition to, or presence of ErbB-related cancer in the subject.

According to another embodiment of the present invention, there is provided a method of diagnosing predisposition to, or prognosis, or presence, or monitoring the progression of, or a responsiveness to treatment of, ErbB-2 related cancer in a subject, the method comprising determining a level of a polypeptide of any of the claims 24, 25, 29, 30, 34, 35, 40-43 or of a polynucleotide encoding said polypeptide in a biological sample obtained from the subject, wherein said level of said polynucleotide or said level of said polypeptide level is correlatable with predisposition to, or presence or absence of the cancer, thereby diagnosing predisposition to, or presence of ErbB-related cancer in the subject.

For any of the above embodiments, the present invention may also optionally be used for detecting metastases in the body (for example, optionally through immunohistochemistry with antibodies according to the present invention as described below).

The present invention successfully addresses the shortcomings of the presently known configurations by providing novel ErbB-2 polypeptides and polynucleotides encoding same, more particularly, to methods and kits using same for treating ErbB-related cancer, such as breast cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-f illustrate the nucleic acid sequences of the novel variants of ErbB-2 of the present invention. FIG. 1a—illustrates the nucleic acid sequence of variant I. FIG. 1b—illustrates the nucleic acid sequence of variant II. FIG. 1c—illustrates the nucleic acid sequence of variant III. FIG. 1d—illustrates the nucleic acid sequence of variant IV. FIG. 1e—illustrates nucleic acid sequence of variant V. FIG. 1f—illustrates nucleic acid sequence of variant VI. Unique regions, start codons and termination codons are highlighted.

FIGS. 2a-f illustrate the amino acid sequences of the novel variants of ErbB-2 of the present invention. FIG. 2a—illustrates the amino acid sequence of variant I. FIG. 2b—illustrates the amino acid sequence of variant II. FIG. 2c—illustrates the amino acid sequence of variant III. FIG. 2d—illustrates the amino acid sequence of variant IV. FIG. 2e—illustrates amino acid sequence of variant V. FIG. 2f—illustrates the amino acid sequence of variant VI. Unique regions are highlighted.

FIG. 3 is a schematic illustration showing exon-intron structure of the ErbB2 variants of the present invention.

FIG. 4 is a schematic illustration showing multiple sequence alignment of the ErbB-2 variants of the present invention against wild type ErbB-2 (GenBank Accession No. gi:10181232), Herstatin (GenBank Accession No. gi_{—}10181232) and a 100 Kda ErbB-2 polypeptide (GenBank Accession No. gi_{—}298693). Alignments was effected using Multalin version 5.4.1 [F. CORPET, 1988, Nucl. Acids Res., 16 (22), 10881-10890] using the following parameters: Symbol comparison table: blosum62; Gap weight: 12; Gap length weight: 2; Consensus levels: high=90% low=50%; Consensus symbols: ! is anyone of IV; $ is anyone of LM; % is anyone of FY; and # is anyone of NDQEBZ.

FIG. 5 is a schematic illustration showing the intron-exon structure of wild type ErbB-2, B2L and B2S. Protein domains are indicated for each sequence. Primers for RT PCR are indicated by arrows. Primers are designated by SEQ ID NO. (see Table 3, below).

FIG. 6 is a a picture of an agarose gel showing the expression of the transcription products of wild-type ErbB-2 and the novel variants B2L and B2S of the present invention in various cell lines, as determined by RT-PCR analysis. mRNA expression level of the ATP synthase 6, house keeping gene, is shown in the lower panel.

FIGS. 7a-b are histograms showing the relative expression of wild-type ErbB-2 (WT), and B2L and B2S variants in normal and tumor derived breast samples as determined by real time RT-PCR using primers for SEQ ID NOs: 47, 50 and 53. Relative expression was normalized to the geometric mean of the relative expression of four housekeeping genes PBGD (GenBank Accession No. BC019323; amplicon—SEQ ID NO: 38), HPRTI (GenBank Accession No. NM_{—}000194; amplicon—SEQ ID NO: 35), G6PD (GenBank Accession No. NM_{—}000402; amplicon—SEQ ID NO: 44) and SDHA (GenBank Accession No. NM_{—}004168; amplicon—SEQ ID NO: 41). The histogram is shown with a smaller scale (FIG. 7A) and with a larger scale (FIG. 7B) for convenience only.

FIGS. 8a-c show time course of small scale expression of B2S in BL21star bacterial cell line from Invitrogen). FIG. 8a shows a Coomassie staining of whole cell extracts at T=0, as well as 1 hour, 2 hours and overnight after transfection. FIG. 8b shows a Western blot analysis of the whole cell extract as above using ErbB-2 specific antibody Ab-20. FIG. 8c shows a Western blot analysis of the whole cell extract as above using the his-tag specific Anti His antibodies [Penta His-HRP conugated antibodies (QIAGEN)]. Note, a band of about 67 kDa corresponds to the B2S variant.

FIGS. 9a-b show Western blot analyses demonstrating the transient expression of the B2L protein in the eukaryotic expression systems, COS7 and in 239T cells. Specific rabbit anti B2L polyclonal antibodies generated according to the teachings of the present invention were used in FIG. 9b, as compared to an ErbB-2 specific antibody Ab-20, which was used in the Western blot analysis demonstrated in FIG. 9a. The lanes were as follows: lane 1=50 ng p185 standard [full length extracellular portion of ErbB-2 gene product, sp185 Her2 from Bender MedSystems GmbH (Austria)]; lane 2=non-concentrated conditioned medium from COS7 cells; lane 3=marker (i.e., molecular weight marker); lane 4=40× concentrated conditioned medium from COS7 cells; lane 5=cell lysate from COS7 cells; lane 6=non-concentrated conditioned medium from 293T cells; and lane 7=40× concentrated conditioned medium from 293T cells. Apart from lanes 1 and 3, all lanes show results from cells transiently expressing B2L. Lane assignments are the same for FIGS. 9a and 9b.

FIG. 10 shows secretion of the B2L variant to tissue culture medium following a large-scale transient transfection. FIG. 10 is a western blot analysis of transiently transfected 293T cells, using the ErbB-2 specific Ab-20 antibody. Lanes are as follows: lane 1=marker; lane 2=p185 standard; lane 3=mock transfected conditioned medium (mock); lane 4=mock transfected 40× concentrated conditioned medium; lane 5=non-concentrated B2L transfected (conditioned) cell medium; lane 6=40× concentrated B2L transfected (conditioned) medium. Similar results were observed with COS7 cells (data not shown), although superior results were observed with 293T cells.

FIGS. 11a-b are Western blot analyses of B2L expression in stably transfected CHO and 293T cells using Ab-20 antibody. FIG. 11a shows the stable expression of recombinant B2L without the His-tag (lane 1=marker, lanes 2 and 3 are conditioned medium, limiting dilution, from transfected clones, lane 4=50 ng of standard p185), while FIG. 11b shows the stable expression of recombinant His-tagged B2L (lane 1=marker, lane 2=293T pool of stably expressed tagged B2L).

FIG. 12a is a Western blot analysis of stable B2L expression in 293T pools (using Ab 20). Lanes are as follows: lane 1=marker; lane 2=p185 standard; lanes 3 and 4 show transient transfections (mock and conditioned medium, respectively); lanes 5-8 show stable transfections from two experiments (lanes 5 and 7 are mock transfections, while lanes 6 and 8 are conditioned medium). Conditioned medium was not concentrated, while an ELISA assay showed that the level of B2L in the conditioned medium was either 6.8 micrograms/ml or 4.6 micrograms/ml, respectively. A Western blot analysis was also performed for stable B2S-his tag expression in 293T, using Ab 20. Positive results were obtained (data not shown).

FIG. 12b is a Western blot analysis of expression of tagged B2S (B2S-His) from stable pool of 293T, by using Ab20 antibody. Lanes are as follows: lane 1=p185 standard; lane 2=marker; lane 3=mock; lane 4=20× concentrated conditioned medium from stable pool of 293T. B2S is shown with an arrow; clearly, it was very strongly expressed in these cells.

FIGS. 13a-b show Western blot analyses demonstrating the time course of B2S variant expression following transient transfection of COS7 cells. In FIG. 13a Ab-20 antibody was used (lane 1=positive control, bacterial lysate of cells expressing B2S; lane 2=marker; lanes 3-5 show 72 hours after transient transfection, 80×, 40× and non-concentrated conditioned medium (after concentration and resuspension of the pellet), respectively; lane 6 shows conditioned medium prior to concentration; lanes 7 and 8 show expression of B2S 48 hours after transient transfection, 40× and non-concentrated (UC) medium respectively; and lanes 9 and 10 show mock transfected cells, 48 hours after mock transfection, 80× and 40× concentrated conditioned medium, respectively). In FIG. 13b specific rabbit polyclonal anti B2S antibodies were used, with the same lane assignments.

FIGS. 14a-b show expression of the recombinant B2S following large scale transient transfection of COS7 cells. In FIG. 14a, Ab-20 antibody was used for the Western blot analysis (1:200 dilution). In FIG. 14b, specific rabbit polyclonal anti B2S antibodies were used for the Western blot analysis. Lane assignments were the same for both blots and were as follows: lane 1=conditioned medium (unconcentrated); lane 2=40× concentrated conditioned medium; and lane 3=40× concentrated medium from mock transfected cells.

FIG. 15 shows the secretion of B2S recombinant protein into the supernatant following transient transfection of 293T or COS7 cells, as detected by Western blotting using the Ab-20 antibody. Lanes are as follows: lane 1=p185 standard (50 ng); lane 2=marker; lanes 3 and 4 show mock transfection of COS7 cells (unconcentrated and 40× concentrated medium, respectively); lanes 3 and 4 show transfection of 293T cells (unconcentrated and 40× concentrated medium, respectively); lanes 5 and 6 show a first experiment with transfection of COS7 cells (unconcentrated and 40× concentrated medium, respectively); and lanes 7 and 8 show a second experiment with transfection of COS7 cells (unconcentrated and 40× concentrated medium, respectively).

FIG. 16 shows a Western blot analysis demonstrating the intracellular expression of the B2S protein after transient transfection of COS7 cells. Ab-20 antibody was used. Lanes 1-3 show dilutions of p185 standard (100, 50 and 10 ng, respectively). Lane 4 shows the marker. Diluted cell lysate (lanes 69, diluted 1:5, 1:10, 1:50 or 1:100 respectively) or original undiluted cell lysate (lane 5) was analyzed as compared to mock (lysate from mock translated cells, lane 10), as indicated. Undiluted cell lysate was shown to contain about 50 ng total B2S protein, in comparison to the dilutions curve for the p185 standard.

FIG. 17 is a Western blot using Ab-20, demonstrating transient expression of His-tagged B2S protein in 293T cells. Lanes are as follows: lane 1=molecular weight marker; lane 2=B2S His-tag non-concentrated conditioned medium (pcDNA3); lane 3=concentrated medium as per lane 2; lane 4=mock transfected using pcDNA3; lane 5=B2S His-tag non-concentrated conditioned medium (pIRESpuro); lane 6=concentrated medium as per lane 5; lane 7=mock transfected using pIRESpuro.

FIGS. 18a-b show the results of immunoaffinity purification of B2L variant protein (stable pool of 293T cells). FIG. 18a shows a Coomassie stained gel, while FIG. 18b shows a Western blot with antibody Ab 20. Lanes are the same for both gels, as follows: lane 1=supernatant from transfected 293T cells; lane 2=marker; lane 3=elution pool I after purification; and lane 4=elution pool II after purification.

FIG. 19 is a Western blot demonstrating the specificity of the polyclonal rabbit anti B2S antibody with regard to B2S expressed in bacterial protein. The Ab-20 was used as a positive control. Lanes are as follows: lane 1=marker; lane 2=rabbit serum diluted 1:1000; lane 3=Ab 20 diluted 1:20 (positive control); lane 4=purified anti-B2S polyclonal antibody (batch 1, elution after load 1); lanes 5 and 6=elution 1, pH 3, batch 2 anti-B2S polyclonal antibody (elution after load 1); lane 7=NaCl elution batch 2 anti-B2S polyclonal antibody (column wash); and lane 8=elution 2, pH 3, batch 2 anti-B2S polyclonal antibody (elution after load 2). An arrow indicates the location of B2S on the blot.

FIG. 20 is a Western blot demonstrating the specificity of the polyclonal rabbit anti B2L antibody with regard to B2L expressed in 293 cells (supernatant, 1 microgram/ml). The Ab-20 was used as a positive control. Lanes are as follows: lane 1=marker; lane 2=rabbit serum diluted 1:1000; lane 3=unbound serum diluted 1:1000 (material that passes through the column without binding); lanes 4-6=purified anti B2L polyclonal antibody (batches 1-3 respectively, related to elutions 1-3, which are elutions of loads 1-3, respectively); and lane 7=Ab 20 (positive control). An arrow indicates the location of B2L on the blot FIGS. 21a-b demonstrate the anti-proliferative activity of B2L on BT474 (FIG. 21a) and SKBR3 (FIG. 21b) cells as determined by an MTT assay. B2L was added directly from conditioned media of stable 293T transfectants with B2L-expressing plasmid. The medium of 293 stable transfectants with empty vector was used as mock (negative control). Herceptin was used as a positive control.

FIGS. 22a-b demonstrate the anti-proliferative activity of B2L on heregulin treated T47D (FIG. 22a) and MCF7 (FIG. 22b) cells, as determined by an MTT assay.

### DESCRIPTION OF THE PREFERRED EMBODIMENTS

The present invention is of novel ErbB-2 transcripts which can be used in diagnosis, prognosis and treatment of ErbB-related cancers, such as breast cancer. The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Human ErbB-2 is an oncogene, located at chromosome 17 q11-q12 and encoding a 185-kD transmembrane glycoprotein. The ErbB-2 protein has an intracellular tyrosine kinase activity and an extracellular domain sharing high homology with other members of the ErbB family of receptor tyrosine kinases [Yamamoto Nature (1986); 319:230-234].

ErbB-2 is expressed in normal and malignant tissues, the latter often exhibiting amplification of the ErbB-2 gene which results in the overexpression of protein having a normal sequence. No mutations have yet been identified in human cancer cells. Although the mechanism of gene amplification is not known, experimental data shows that ErbB-2 acts as a potent oncogene in vitro [Di Fiore Proc Natl Acad Sci USA 1992; 89:10578-10582] and in vivo [Guy J Biol Chem 1996;271:7673-7678; Slamon Science 1987;235:177-182]. A correlation has been noted between ErbB-2 gene amplification and/or protein overexpression and poor disease-free survival. ErbB-2 overexpression has also been associated with resistance to chemotherapy and hormone therapy [reviewed by Nahta The Oncologist, Vol. 8, No. 1, 5-17, (2003)].

For these reasons development of reliable assays for assessing ErbB-2 levels in a given cancer tissue or cell and identification of novel anti-ErbB-2 drugs are at the center of cancer research.

While reducing the present invention to practice the present inventors uncovered novel isoforms of ErbB-2 polypeptide, which can be used to diagnose predisposition to, and design therapeutic tools for ErbB-related types of pathologies.

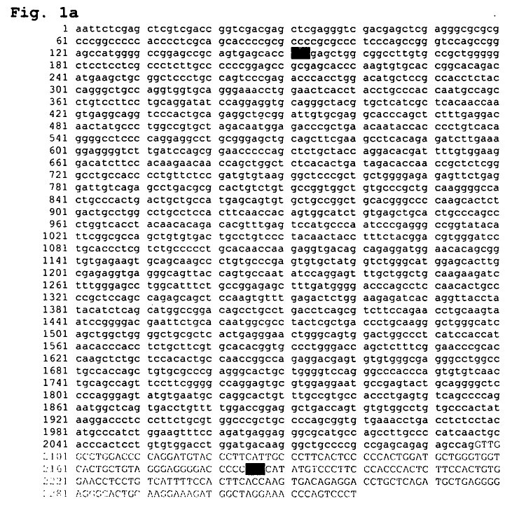

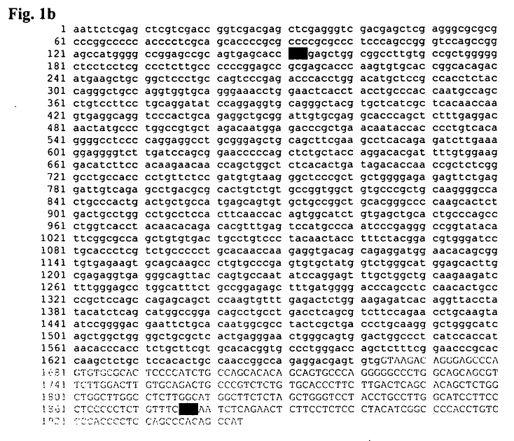

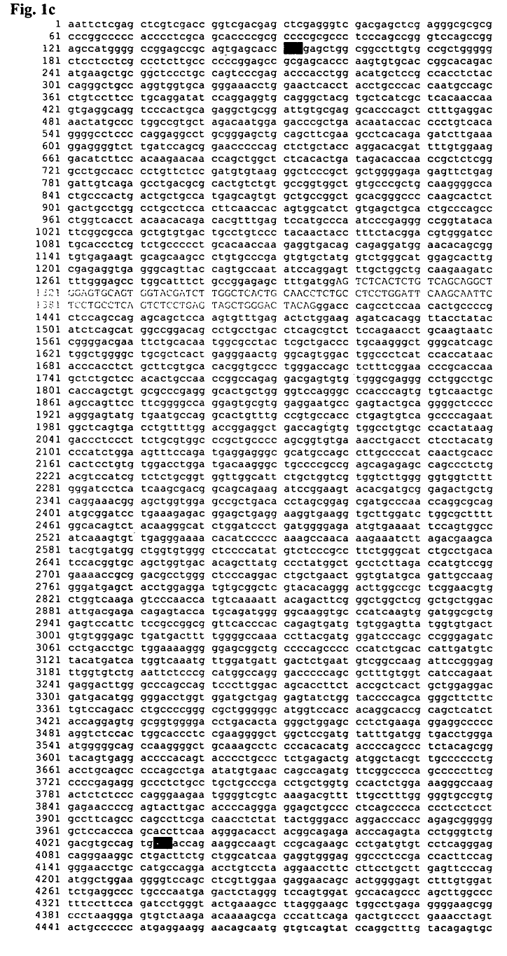

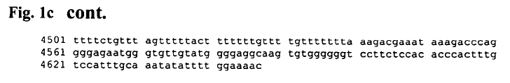

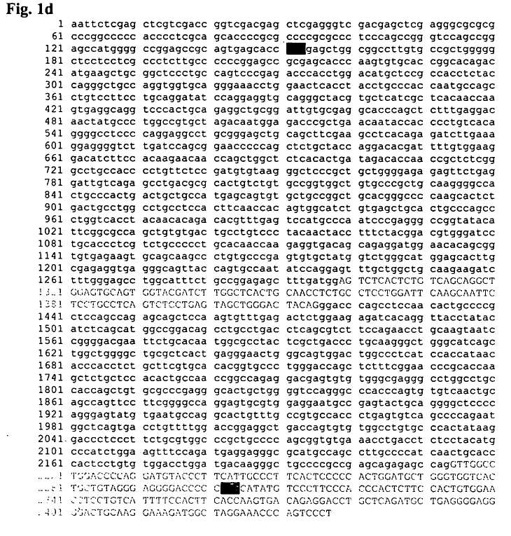

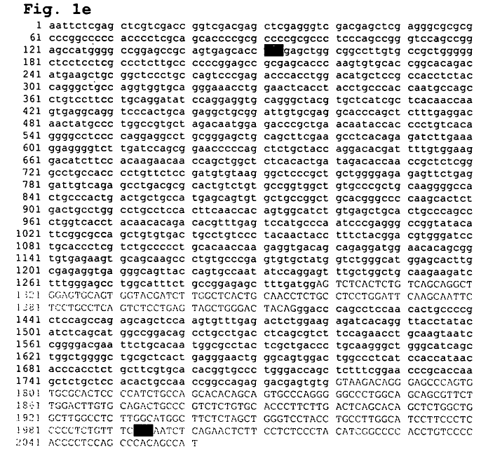

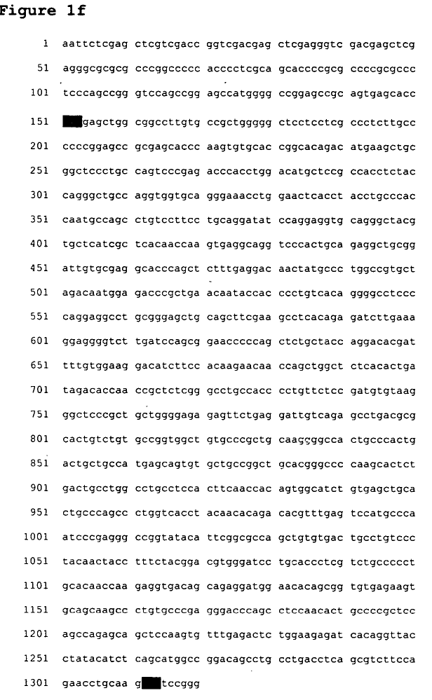

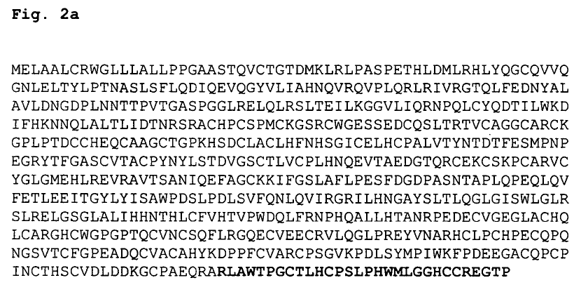

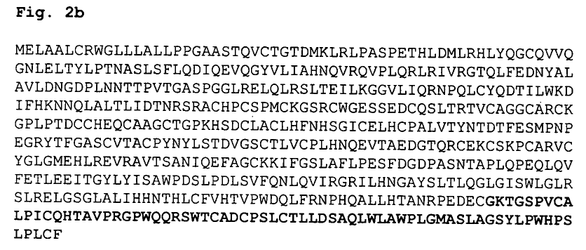

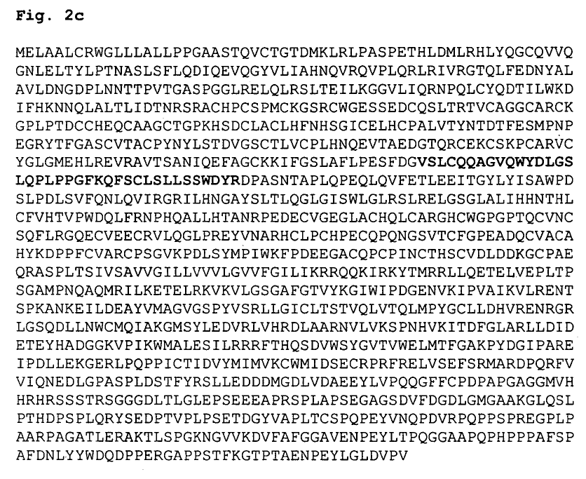

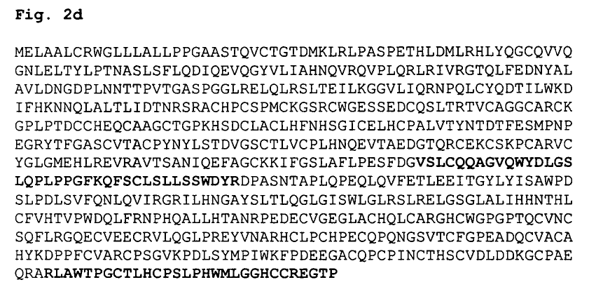

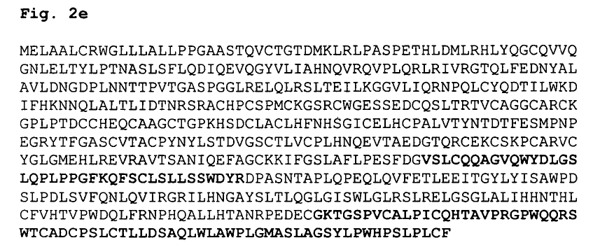

A number of variants according to the present invention (including without limitation Variant I [SEQ ID NOs: 1 (polynucleotide) and 2 (polypeptide)], variant II (SEQ ID NOs: 3 and 4) and variant VI (SEQ ID NOs: 25 and 26)) are secreted polypeptides, which result from alternative splicing of the ErbB-2 gene. Variant I includes the complete extracellular domain (ECD) of native ErbB-2 and a unique sequence of 30 amino acids at the C-terminus of the protein and thus is 575 amino acid long. Variant II mRNA encodes a nearly complete ECD (excluding the carboxyl terminal Furin) and includes a C-terminal sequence of 71 amino acids unique to this variant (see FIGS. 1a-e, 2a-e and 5). Variant VI (SEQ ID NOs: 25 and 26) results from exon 9 skipping, an alteration of the open reading frame past this exon and termination at a stop codon located on exon 11. Variant VI mRNA encodes a polypeptide having 387 amino acids, which includes a stretch of 340 shared by wild type ErbB-2 and a stretch of 47 amino acids unique to this variant (see FIGS. 1f and 2f, SEQ ID NO:28).

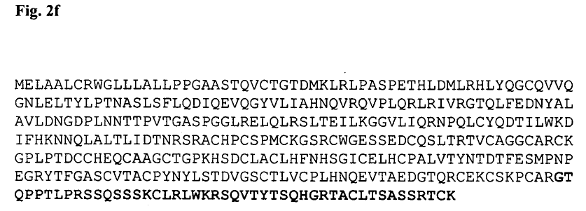

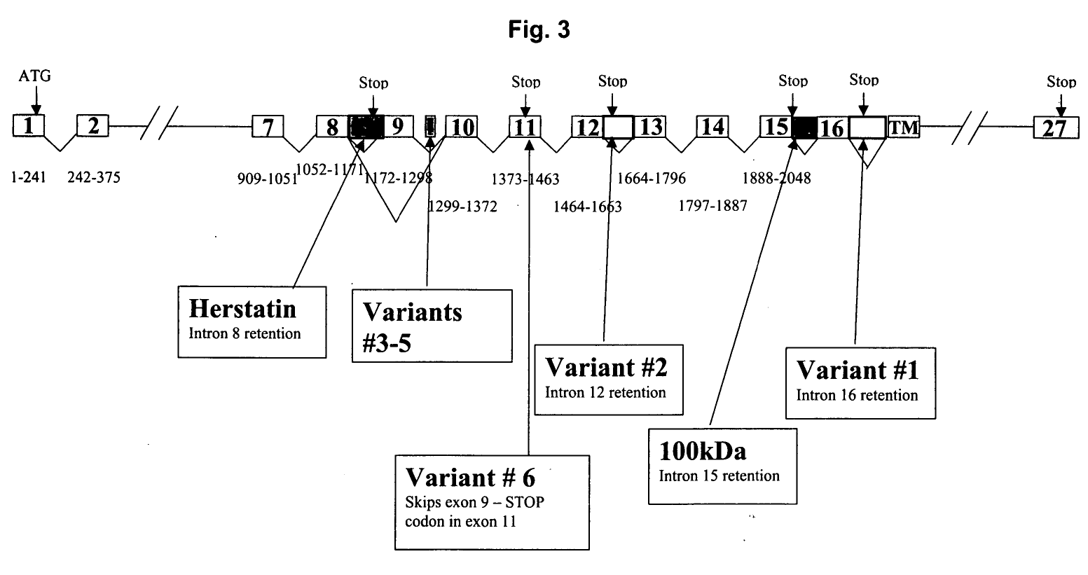

Examination of the 5′ and 3′ junctions of the unique sequence region of variants I and II of the present invention including the new exon reveals consensus splice donor and acceptor sites and includes a pyrimidine tract and potential branchpoint adenine residues near the 3′end of the insert sequence (FIG. 3).

The three variants (I, II and VI) include a signal peptide of 27 amino acids at the amino termini of these proteins. The 74.5 Kda variant I has a calculated pI of 5.94, while the 63.15 KDa variant II has a calculated pI of 6.08. Expression analysis showed that variants I and II are ubiquitously expressed and exhibit temporal and spatial expression patterns which are similar to that of the full length ErbB-2 (see Example 1 of the Examples section).

Variant VI was uncovered by PCR analysis of the lung cancer cell line Calu-3 (ATCC Accession No: HTB-55) using the primers set foth by SEQ ID NOs: 29 and 30.

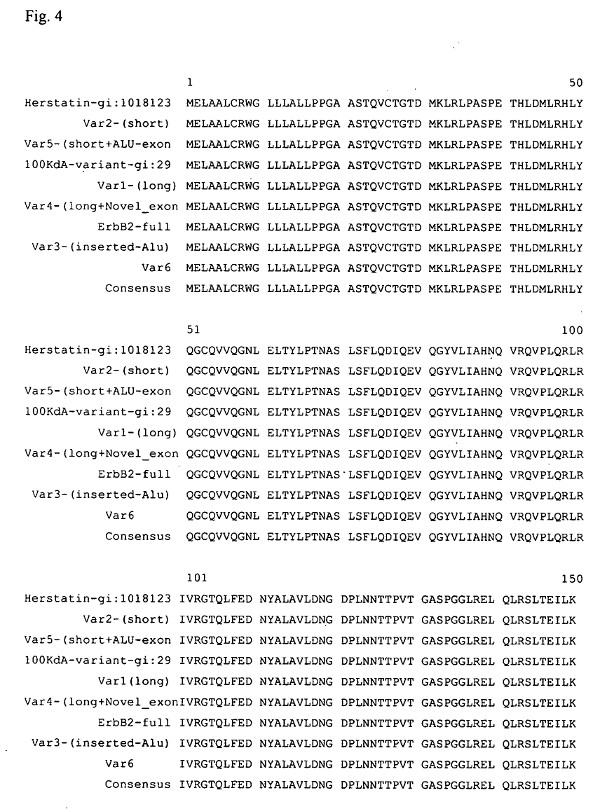

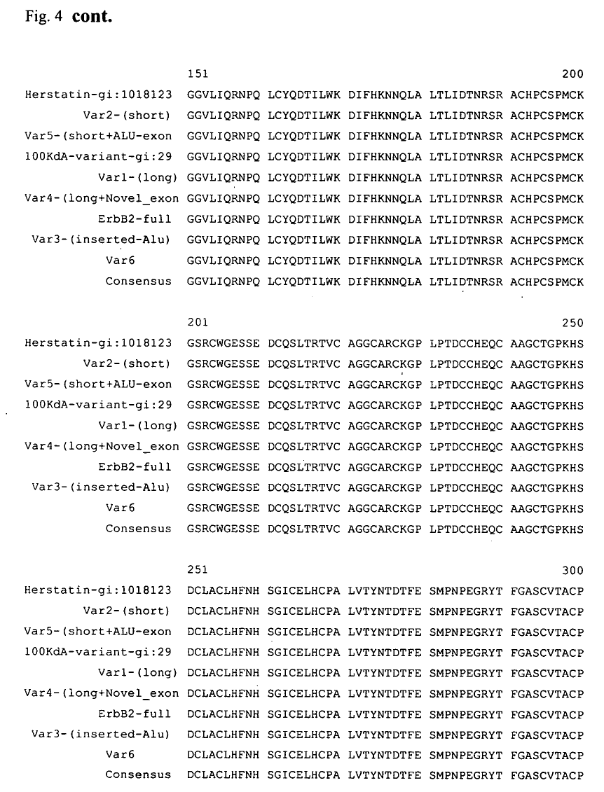

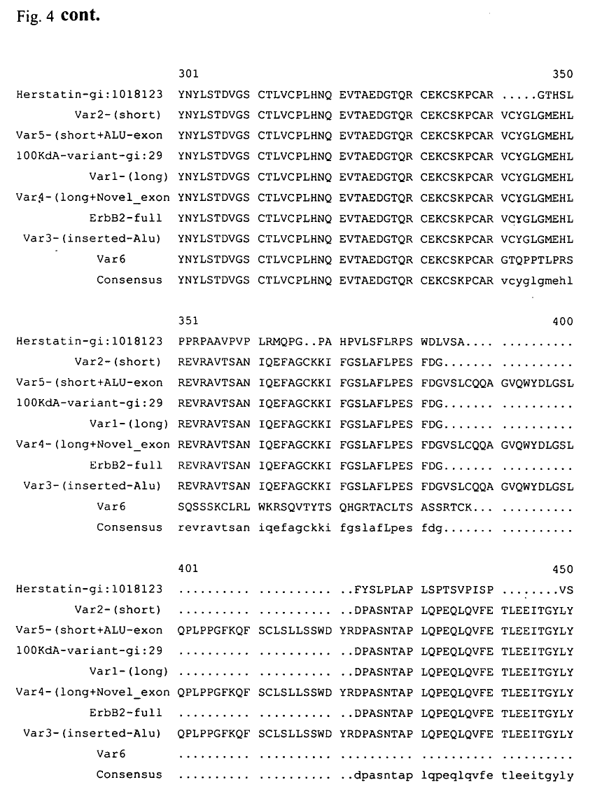

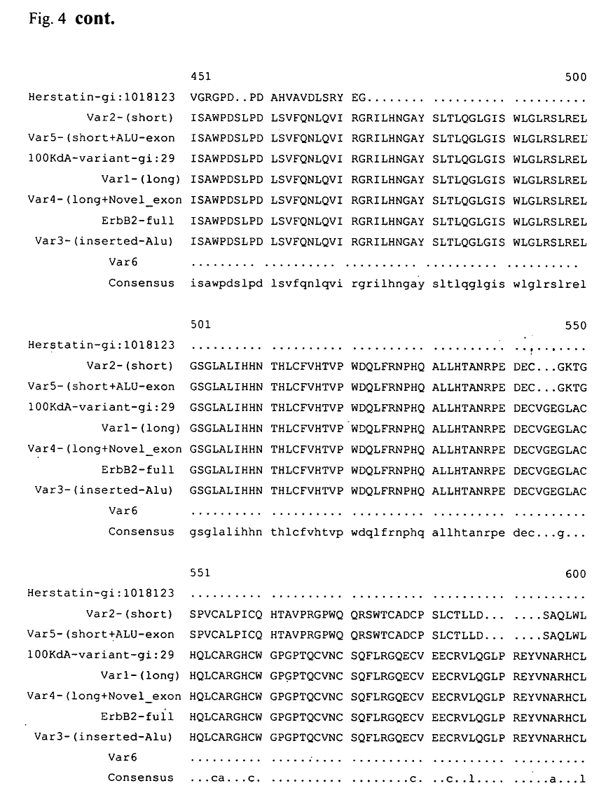

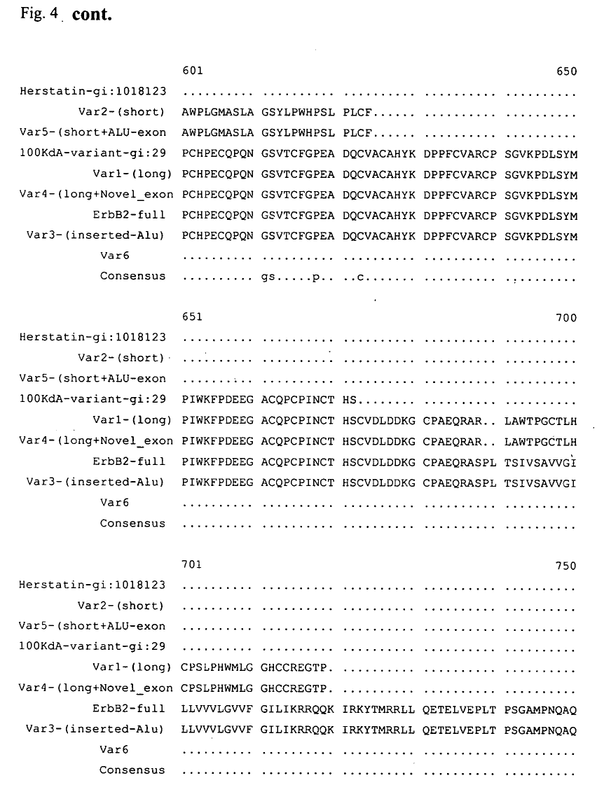

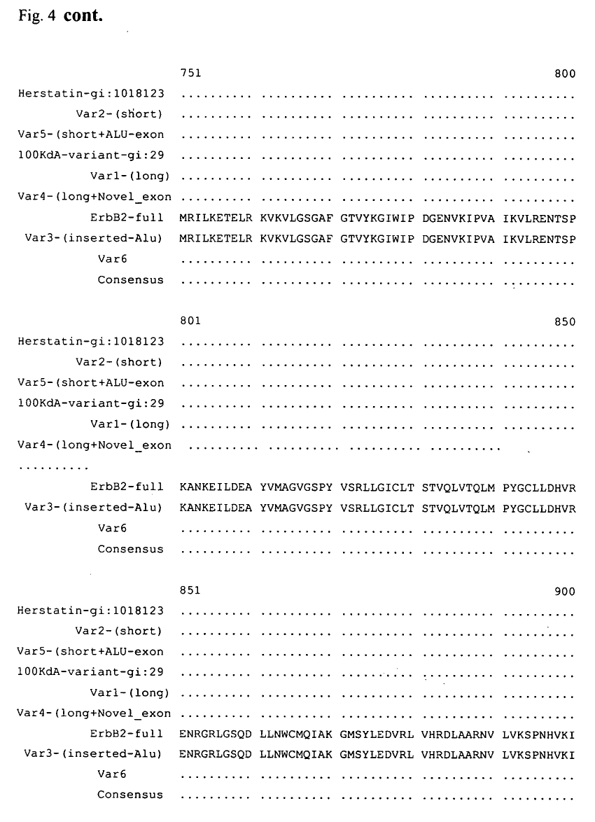

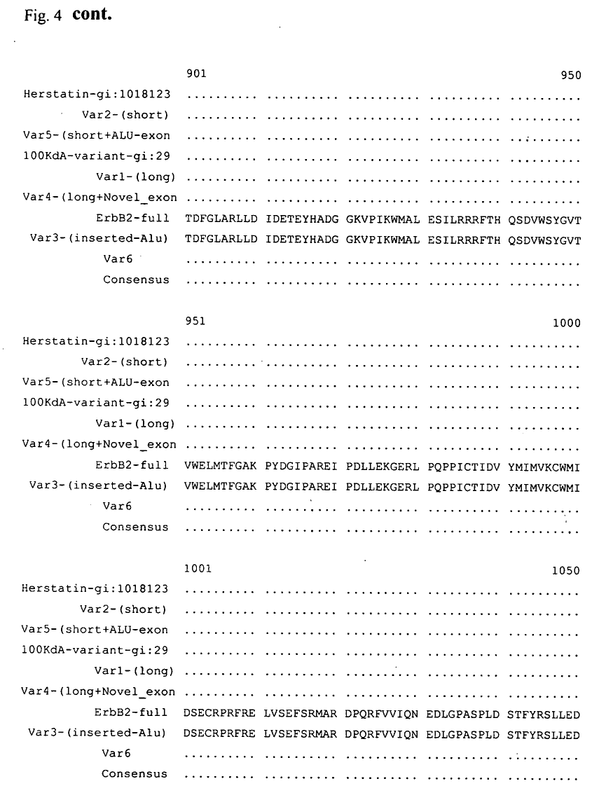

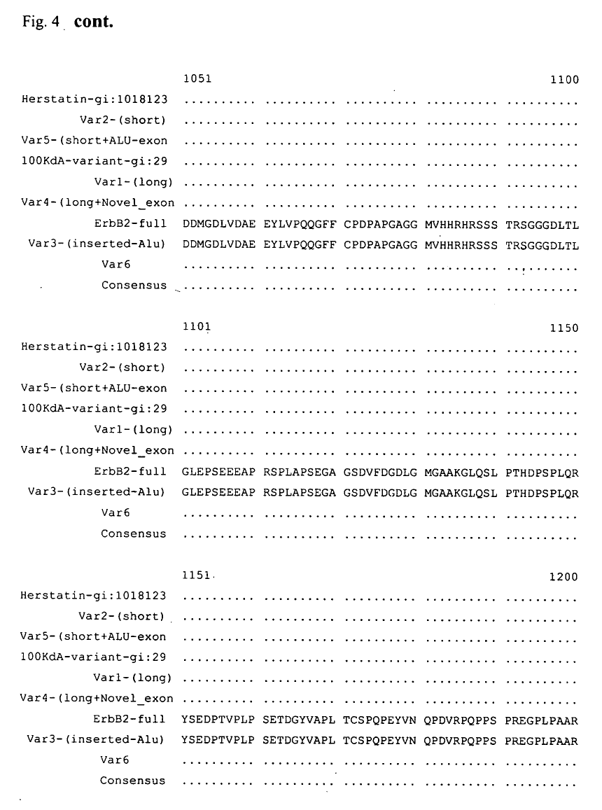

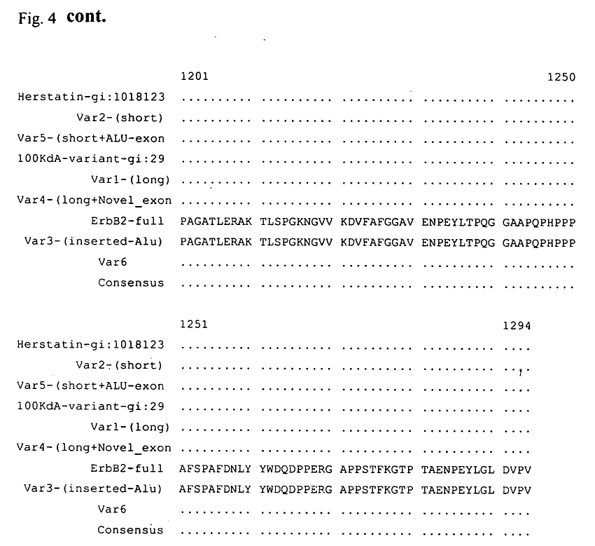

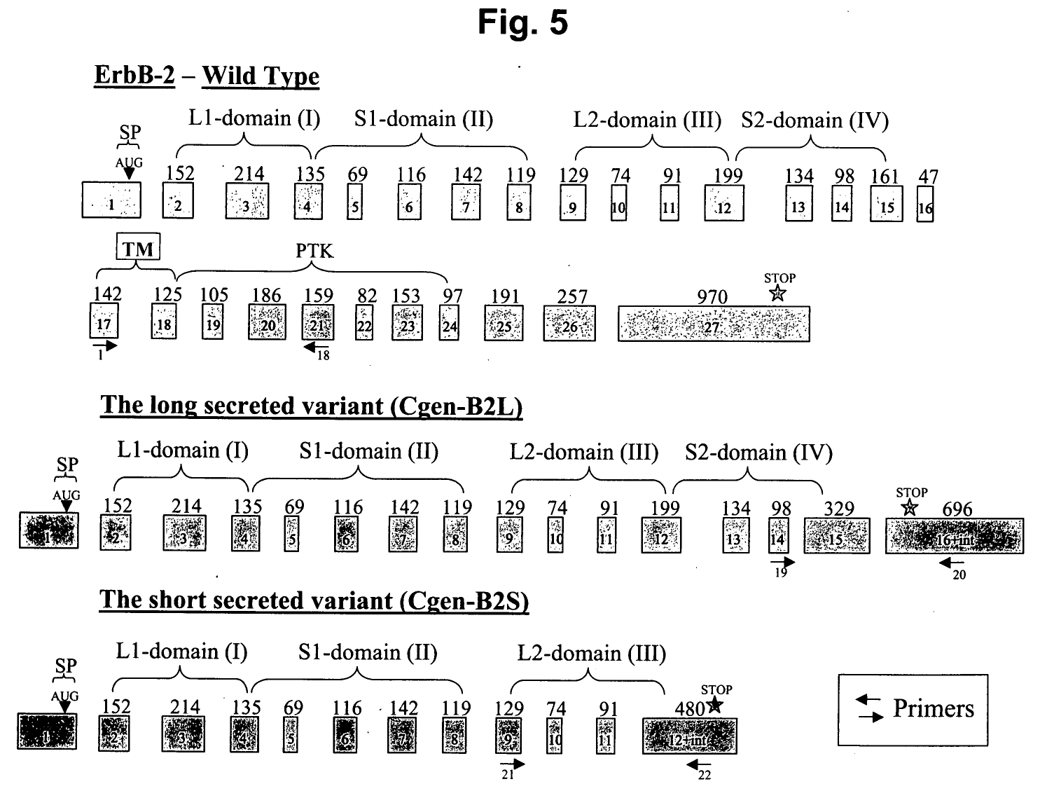

While further reducing the present invention to practice the present inventors also uncovered a novel exon of ErbB-2 which is positioned between exon 9 and 10 of the ErbB-2 gene, giving rise to membrane-anchored as well as secreted ErbB-2 transcripts (i.e., variants III-V as follows: SEQ ID NOs: 9-10 (var III), 13-14 (var IV) and 15-16 (var V), respectively, FIGS. 1a-e and 2a-e), which include an inframe insertion of 39 amino acids centrally located at the extracellular domain of ErbB-2 (FIGS. 2-4).

Without being bound by theory, since the secreted ErbB-2 isoforms of the present invention are devoid of a transmembrane and intracellular kinase domain, yet retain the ability to dimerize, they may prevent the formation of ErbB functional dimers required for transphosphorylation and receptor activation, simply by sequestration of ErbB-2 binding partners i.e., ErbB-1 (EGFR), ErbB-3 and ErbB-4, thereby exerting a dominant negative effect. Alternatively the newly discovered ErbB-2 isoforms of the present invention may compete with membrane-bound ErbB-2 on the binding to a still unknown ligand.

The present invention features various embodiments, including polypeptides and peptides, and nucleic acid fragments, as well as antibodies, primers, amplicons, hybridizing oligonucleotides, and compositions and methods of use thereof.

Amino Acid Sequences According to the Present Invention

The present invention includes polypeptides, fragments thereof and peptides that are related to ErbB-2 variants.

As used herein the phrase “an ErbB-2 polypeptide” refers to a variant of a “wild-type” (WT) ErbB-2 protein (GenBank Accession No: gi: AAA75493).

Preferably the ErbB-2 polypeptide according to this aspect of the present invention refers to a secreted ErbB-2 polypeptide which preferably retains an intact signal peptide to allow secretion but is devoid of a transmembrane domain of the protein. Such secreted polypeptides may result from alternative splicing of the ErbB-2 gene or from shedding of the extracellular portion [see Doherty (1999) Proc. Natl. Acad. Sci. U.S.A. 96, 10869-10874 and Lin and Clinton, Oncogene 6:639-643, (1991); Streckfus Clinical Cancer Research, 6:2363-2370 (2000); Langton Cancer Res, (1991), 51:2593-2598].

Thus, the present invention encompasses polypeptides encoded by the novel ErbB-2 variants of the present invention or active portions thereof. The amino acid sequences of these novel polypeptides are set forth in SEQ ID NO:2, 4, 5, 6, 10, 11, 14, 16, 26 or 28. The present invention also encompasses homologues of these polypeptides, such homologues can be at least 50%, at least 55%, at least 60%, at least 63%, at least 65%, at least 69%, at least 70%, at least 71%, at least 73%, at least 75%, at least 77%, at least 80%, at least 83%, at least 85%, at least 95% or more say 100% homologous to SEQ ID NOs: 2, 4, 5, 6, 10, 11, 14, 16, 26 or 28. The present invention also encompasses fragments of the above described polypeptides and polypeptides having mutations, such as deletion, insertion or substitution of one or more amino acids, either naturally occurring or man induced, either randomly or in a targeted fashion.

According to a preferred embodiment of this aspect of the present invention the polypeptide includes an amino acid sequence which is at least 64%, at least 70%, at least 71%, at least 73%, at least 77%, at least 80%, at least 85%, at least 95% or more say 100% identical to SEQ ID NO:5, as determined using BlastP software of the National Center of Biotechnology Information (NCBI) using default parameters.

According to another preferred embodiment of this aspect of the present invention the polypeptide includes an amino acid sequence at least 58%, at least 60%, at least 65%, at least 69%, at least 70%, at least 75%, at least 80%, at least 85%, at least 95% or more say 100% identical to SEQ ID NO:6, as determined using BlastP software of the National Center of Biotechnology Information (NCBI) using default parameters.

According to yet another preferred embodiment of this aspect of the present invention the polypeptide includes an amino acid sequence at least 85%, at least 87%, at least 89%, at least 91%, at least 93%, at least 95% or more say 100% homologous to SEQ ID NO:10, 14 or 16 (variant III, IV or V, respectively), as determined using BlastP software of the National Center of Biotechnology Information (NCBI) using default parameters.

According to still another preferred embodiment of this aspect of the present invention the polypeptide includes an amino acid sequence at least 85%, at least 87%, at least 89%, at least 91%, at least 93%, at least 95% or more say 100% identical to SEQ ID NO:11, as determined using BlastP software of the National Center of Biotechnology Information (NCBI) using default parameters.

According to an additional preferred embodiment of this aspect of the present invention the polypeptide includes an amino acid sequence at least 63%, at least 65%, at least 70%, at least 73%, at least 75%, at least 80%, at least 83%, at least 85%, at least 87%, at least 89%, at least 91%, at least 93%, at least 95% or more say 100% homologous to SEQ ID NO:28 (portion of variant VI), as determined using the protein Blast search software for short, nearly exact matches of the National Center of Biotechnology Information (NCBI) using default parameters.

As used hereinabove the phrase “active portion” refers to an amino acid sequence portion which is capable of displaying one or more functions of the ErbB-2 polypeptides of the present invention. Examples include but are not limited to ligand binding, receptor dimerization, signaling and antibody specific recognition.

According to one preferred embodiment of this aspect of the present invention the active portion of the polypeptide includes amino acid coordinates 649-678 of SEQ ID NO:2 (i.e., SEQ ID NO:5), which is encoded by nucleotide coordinates 2097-2320 of SEQ ID NO:1 (i.e., SEQ ID NO:7).

According to another preferred embodiment of this aspect of the present invention, the active portion of the polypeptide includes amino acid coordinates 505-575 of SEQ ID NO:4 (i.e., SEQ ID NO:6), which is encoded by nucleotide coordinates 1664-1944 of SEQ ID NO:3 (i.e., SEQ ID NO:8).

According to yet another preferred embodiment of this aspect of the present invention, the active portion of the polypeptide of this aspect of the present invention preferably includes amino acid coordinates 384-422 of SEQ ID NO:10 (i.e., SEQ ID NO:11), which is encoded by nucleotide coordinates 1299-1415 of SEQ ID NO:9 (i.e., SEQ ID NO:12).

The present invention encompasses the polypeptides of the splice variants of ErbB-2, or the fragments thereof, including but not limited to the following:

1. An isolated polypeptide of ErbB-2 variant I (SEQ ID NO: 2), comprising a first amino acid sequence being at least 90% homologous to amino acids 1-648 of wild type ErbB-2 (GenBank Accession No: gi: AAA75493), which also encompasses amino acids 1-648 of the sequence as set forth in SEQ ID NO:2, and a second amino acid sequence being at least about 70%, optionally at least about 80%, preferably at least about 85%, more preferably at least about 90% and most preferably at least about 95% homologous to a polypeptide having the sequence RLAWTPGCTLHCPSLPHWMLGGHCCREGTP (SEQ ID NO: 5), wherein the first and the second amino acid sequences are contiguous and in a sequential order.

2. An isolated polypeptide of a tail of ErbB-2 variant I (SEQ ID NO: 2), comprising a polypeptide having the sequence RLAWTPGCTLHCPSLPHWMLGGHCCREGTP (SEQ ID NO: 5).

As used herein a “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the wild type or known protein, while at least a second portion of the variant comprises the tail.

3. A unique edge portion of SEQ ID NO: 2, comprising a polypeptide being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 5).

As used herein “an edge portion” refers to a connection between two portions of a splice variant according to the present invention that were not joined in the wild type or known protein. An edge may optionally arise due to a join between the above “wild type” portion of a variant and the tail, for example, and/or may occur if an internal portion of the wild type sequence is no longer present, such that two portions of the sequence are now contiguous in the splice variant that were not contiguous in the known protein.

4. A bridge portion of SEQ ID NO:2, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AR, having a structure as follows (numbering according to SEQ ID NO:2): a sequence starting from any of amino acid number 648-x to 648; and ending at any of amino acid numbers 649+((n−2)−x), in which x varies from 0 to n−2, with the proviso that the value ((n−2)−x) is not allowed to be larger than 29.

For example, for peptides of 10 amino acids (such that n=10), the starting position could be as “early” in the sequence as amino acid number 640 if x=n−2=8 (ie 640=648−8), such that the peptide would end at amino acid number 649 (649+(8−8=0)). On the other hand, the peptide could start at amino acid number 648 if x=0 (ie 648=648−0), and could end at amino acid 657 (649+(8−0=8)).

The bridge portion of SEQ ID NO:2 above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: ARLA, RARL, QRAR. All peptides feature AR as a portion thereof. Peptides of from about five to about nine amino acids could optionally be similarly constructed.

5. An isolated polypeptide of ErbB-2 variant II (SEQ ID NO: 4), comprising a first amino acid sequence being at least 90% homologous to amino acids 1-504 of wild type ErbB-2 (GenBank Accession No: gi: AAA75493), which also encompasses amino acids 1-504 of the sequence as set forth in SEQ ID NO: 4, and a second amino acid sequence being at least about 70%, optionally at least about 80%, preferably at least about 85%, more preferably at least about 90% and most preferably at least about 95% homologous to a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6), wherein the first and the second amino acid sequences are contiguous and in a sequential order.

6. An isolated polypeptide of a tail of ErbB-2 variant II (SEQ ID NO: 4), comprising a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6).

7. A unique edge portion of SEQ ID NO: 4, comprising a polypeptide being at least 70%, optionally at least about 80%, preferably at least about 85%, more preferably at least about 90% and most preferably at least about 95% homologous to the sequence GKTGSPVCALPICQHTAVPRGPWQQRSWTCADCPSLCTLLDSAQLWLAWPL GMASLAGSYLPWHPSLPLCF (SEQ ID NO: 6).

8. A bridge portion of SEQ ID NO:4, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise CG, having a structure as follows (numbering according to SEQ ID NO:4): a sequence starting from any of amino acid number 504-x to 504; and ending at any of amino acid numbers 505+((n−2)−x), in which x varies from 0 to n−2, with the proviso that the value ((n−2)−x) is not allowed to be larger than 70.

For example, for peptides of 10 amino acids (such that n=10), the starting position could be as “early” in the sequence as amino acid number 496 if x=n−2=8 (ie 496=504−8), such that the peptide would end at amino acid number 505 (505+(8−8=0)). On the other hand, the peptide could start at amino acid number 504 if x=0 (ie 504=504-0), and could end at amino acid 513 (505+(8−0=8)).

The bridge portion of SEQ ID NO: 4 above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: CGKT, ECGK, DECG. All peptides feature CG as a portion thereof. Peptides of from about five to about nine amino acids could optionally be similarly constructed.

9. An isolated polypeptide of ErbB-2 variant III (SEQ ID NO: 10), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 10, an amino acid sequence being at least about 70%, optionally at least about 80%, preferably at least about 85%, more preferably at least about 90% and most preferably at least about 95% homologous to a bridging polypeptide having the sequence VSLCQQAGVQWYDLGSLQPLPPGFKQFSCLSLLSSWDYR (SEQ ID NO: 11), and a second amino acid sequence being at least 90% homologous to amino acids 384-1255 of wild type ErbB-2 (GenBank Accession No: gi: AAA75493), which also encompasses amino acids 423-1294 of the sequence as set forth in SEQ ID NO: 10, wherein the first amino acid is contiguous to the bridging polypeptide and the second amino acid sequence is contiguous to the bridging polypeptide, and wherein the first amino acid, the bridging polypeptide and the second amino acid sequence are in a sequential order.

10. An isolated polypeptide encoding for an edge portion of ErbB-2 variant III (SEQ ID NO: 10), comprising a polypeptide having the sequence VSLCQQAGVQWYDLGSLQPLPPGFKQFSCLSLLSSWDYR (SEQ ID NO: 11).

11. A unique edge portion of SEQ ID NO: 10, comprising a polypeptide being at least 70%, optionally at least about 80%, preferably at least about 85%, more preferably at least about 90% and most preferably at least about 95% homologous to the sequence VSLCQQAGVQWYDLGSLQPLPPGFKQFSCLSLLSSWDYR (SEQ ID NO: 11).

12. A bridge portion of SEQ ID NO: 10, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GV, having a structure as follows (numbering according to SEQ ID NO:10): a sequence starting from any of amino acid number 383-x to 383; and ending at any of amino acid number ending at any of amino acid numbers 384+((n−2)−x), in which x varies from 0 to n−2.

For example, for peptides of 10 amino acids (such that n=10), the starting position could be as “early” in the sequence as amino acid number 375 if x=n-2=8 (ie 375=383−8), such that the peptide would end at amino acid number 384 (384+(8−8=0)). On the other hand, the peptide could start at amino acid number 383 if x=0 (ie 383=383-0), and could end at amino acid 392 (384+(8−0=8)).

The bridge portion above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: GVSL, DGVS, FDGV. All peptides feature GV as a portion thereof. Peptides of from about five to about nine amino acids could optionally be similarly constructed.

13. A second bridge portion of SEQ ID NO: 10, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RD, having a structure as follows (numbering according to SEQ ID NO:10): a sequence starting from any of amino acid number 421-x to 421; and ending at any of amino acid number ending at any of amino acid numbers 422+((n−2)−x), in which x varies from 0 to n−2.

For example, for peptides of 10 amino acids (such that n=10), the starting position could be as “early” in the sequence as amino acid number 413 if x=n−2=8 (ie 413=421−8), such that the peptide would end at amino acid number 422 (422+(8−8=0)). On the other hand, the peptide could start at amino acid number 421 if x=0 (ie 421=421−0), and could end at amino acid 430 (422+(8−0=8)).

The bridge portion of SEQ ID 10 above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: DYRD, YRDP, RDPA. All peptides feature RD as a portion thereof. Peptides of from about five to about nine amino acids could optionally be similarly constructed.

14. An isolated polypeptide of ErbB-2 variant IV (SEQ ID NO: 14), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 14, an amino acid sequence being at least about 70%, optionally at least about 80%, preferably at least about 85%, more preferably at least about 90% and most preferably at least about 95% homologous to a polypeptide having the sequence VSLCQQAGVQWYDLGSLQPLPPGFKQFSCLSLLSSWDYR (SEQ ID NO: 11), a second amino acid sequence being at least 90% homologous to amino acids 384-648 of wild type ErbB-2 (GenBank Accession No: gi: AAA75493), which also encompasses amino acids 423-687 of the sequence as set forth in SEQ ID NO: 14, followed by an amino acid sequence tail being at least about 70%, optionally at least about 80%, preferably at least about 85%, more preferably at least about 90% and most preferably at least about 95% homologous to a polypeptide having the sequence RLAWTPGCTLHCPSLPHWMLGGHCCREGTP (SEQ ID NO: 5), and wherein the first amino acid sequence, the bridging polypeptide, the second amino acid and the amino acid sequence tail are contiguous and in a sequential order.

15. An isolated polypeptide of ErbB-2 variant V (SEQ ID NO: 16), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 16, an amino acid sequence being at least about 70%, optionally at least about 80%, preferably at least about 85%, more preferably at least about 90% and most preferably at least about 95% homologous to a polypeptide having the sequence VSLCQQAGVQWYDLGSLQPLPPGFKQFSCLSLLSSWDYR (SEQ ID NO: 11), a second amino acid sequence being at least 90% homologous to amino acids 384-804 of wild type ErbB-2 (GenBank Accession No: gi: AAA75493), which also encompasses amino acids 423-543 of the sequence as set forth in SEQ ID NO: 16, followed by an amino acid sequence tail being at least about 70%, optionally at least about 80%, preferably at least about 85%, more preferably at least about 90% and most preferably at least about 95% homologous to a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6), wherein the first amino acid is contiguous to the bridging polypeptide and the second amino acid sequence is contiguous to the bridging polypeptide, and wherein the first amino acid, the bridging polypeptide, the second amino acid and the amino acid sequence tail are in a sequential order.

16. An isolated polypeptide of ErbB-2 variant VI (SEQ ID NO: 26), comprising a first amino acid sequence being at least 90% homologous to amino acids 1-340 of wild type ErbB-2 (GenBank Accession No: gi: AAA75493), which also encompasses amino acids 1-340 of the sequence as set forth in SEQ ID NO:26, and a second amino acid sequence being at least about 70%, optionally at least about 80%, preferably at least about 85%, more preferably at least about 90% and most preferably at least about 95% homologous to a polypeptide having the sequence GTQPPTLPRSSQSSSKCLRLWKRSQVTYTSQHGRTACLTSASSRTCK (SEQ ID NO: 28), wherein the first and the second amino acid sequences are contiguous and in a sequential order.

17. An isolated polypeptide of a tail of ErbB-2 variant VI (SEQ ID NO: 26), comprising a polypeptide having the sequence GTQPPTLPRSSQSSSKCLRLWKRSQVTYTSQHGRTACLTSASSRTCK (SEQ ID NO: 28).

18. A unique edge portion of SEQ ID NO: 26, comprising a polypeptide being at least 70%, optionally at least about 80%, preferably at least about 85%, more preferably at least about 90% and most preferably at least about 95% homologous to the sequence GTQPPTLPRSSQSSSKCLRLWKRSQVTYTSQHGRTACLTSASSRTCK (SEQ ID NO: 28).

19. A bridge portion of SEQ ID NO:26, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RG, having a structure as follows (numbering according to SEQ ID NO:26): a sequence starting from any of amino acid number 340-x to 340; and ending at any of amino acid number ending at any of amino acid numbers 341+((n−2)−x), in which x varies from 0 to n−2, with the proviso that the value ((n−2)−x) is not allowed to be larger than 46.

For example, for peptides of 10 amino acids (such that n=10), the starting position could be as “early” in the sequence as amino acid number 332 if x=n−2=8 (ie 332=340−8), such that the peptide would end at amino acid number 341 (341+(8−8=0)). On the other hand, the peptide could start at amino acid number 340 if x=0 (ie 340=340−0), and could end at amino acid 349 (341+(8−0=8)).

The bridge portion of SEQ ID NO:26 above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: CARG, ARGT, RGTQ. All peptides feature RG as a portion thereof. Peptides of from about five to about nine amino acids could optionally be similarly constructed.

It will be appreciated that peptides of the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (Table 2) which can be used with the present invention.

TABLE 1

| Amino Acid | [symbol] hree-Letter Abbreviation | One-letter Symbol |
|---|---|---|
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| Carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo | Mhphe |
| | | [symbol] enylalanine | |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α- | Mhphe |
| L-α-methylisoleucine | Mile | ethylhom [symbol] ethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| [symbol] ylamino)cyclopropane | | | |

Since the peptides of the present invention are preferably utilized in therapeutics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the peptides of the present invention are desired, the peptides of the present invention can be generated using recombinant techniques such as described in Example 4 of the Examples section and by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Designed Peptides According to the Present Invention

As mentioned hereinabove, the inhibitory properties (i.e., inactivation of ErbB0-2 signaling cascade) of the ErbB-2 polypeptides of the present invention can be used in a number of therapeutic applications. In such applications it is highly desirable to employ the minimal and most efficacious peptide regions which still exert inhibitory function. Identification of such peptide regions can be effected using various approaches, including, for example, display techniques.

Thus, according to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12;274(4):622-34; Giebel L B et al “Screening of cyclic peptide phage libraries identifies ligands that bind streptavidin with high affinities” Biochemistry 1995 Nov. 28;34(47):15430-5; Davies E L et al., “Selection of specific phage-display antibodies using libraries derived from chicken immunoglobulin genes” J Immunol Methods 1995 Oct. 12;186(1):125-35; Jones C RT al. “Current trends in molecular recognition and bioseparation” J Chromatogr A 1995 Jul. 14;707(1):3-22; Deng S J et al. “Basis for selection of improved carbohydrate-binding single-chain antibodies from synthetic gene libraries” Proc Natl Acad Sci USA 1995 May 23;92(11):4992-6; and Deng S J et al. “Selection of antibody single-chain variable fragments with improved carbohydrate binding by phage display” J Biol Chem 1994 Apr. 1;269(13):9533-8, which are incorporated herein by reference.

Alternatively, peptide sequences capable of inactivating the ErbB signaling cascade can be uncovered using computational biology. Software programs useful for displaying three-dimensional structural models, such as RIBBONS (Carson, M., 1997. Methods in Enzymology 277, 25), 0 (Jones, TA. et al., 1991. Acta Crystallogr. A47, 110), DINO (DINO: Visualizing Structural Biology (2001) http://www.dino3d.org); and QUANTA, INSIGHT, SYBYL, MACROMODE, ICM, MOLMOL, RASMOL and GRASP (reviewed in Kraulis, J., 1991. Appl Crystallogr. 24, 946) can be utilized to model interactions between the polypeptides of the present invention and prospective peptide sequences to thereby identify peptides which display the highest probability of binding for example to an ErbB protein (e.g., ErbB-3). Computational modeling of protein-peptide interactions has been successfully used in rational drug design, for further detail, see Lam et al., 1994. Science 263, 380; Wlodawer et al., 1993. Ann Rev Biochem. 62, 543; Appelt, 1993. Perspectives in Drug Discovery and Design 1, 23; Erickson, 1993. Perspectives in Drug Discovery and Design 1, 109, and Mauro M J. et al, 2002. J Clin Oncol. 20, 325-34.

Nucleic Acid Sequences According to the Present Invention

The present invention includes polynucleotides, fragments thereof and oligonucleotides that are related to the ErbB-2 variants of the present invention.

The present invention features an isolated polynucleotide (i.e., Variant I) having a nucleic acid sequence encoding at least an active portion of an ErbB-2 polypeptide including an amino acid sequence being at least 65%, at least 70%, at least 71%, at least 73%, at least 77%, at least 80%, at least 85%, at least 95% or more say 100% homologous to SEQ ID NO:5, as determined using BlastP software of the National Center of Biotechnology Information (NCBI) using default parameters.

As used herein the phrase “a n isolated polynucleotide” refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

According to a preferred embodiment of this aspect of the present invention the polypeptide encoded by the polynucleotide of this aspect of the present invention includes an amino acid sequence which is at least 64%, at least 70%, at least 71%, at least 73%, at least 77%, at least 80%, at least 85%, at least 95% or more say 100% identical to SEQ ID NO:5, as determined using BlastP software of the National Center of Biotechnology Information (NCBI) using default parameters.

According to another preferred embodiment of this aspect of the present invention the nucleic acid sequence is as set forth in SEQ ID NO:1, 7, 8 or 13.

Preferably, the polynucleotide according to this aspect of the present invention encodes a polypeptide, which is set forth in SEQ ID NO: 2, 5, 6 or 14.

The present invention also features an isolated polynucleotide having a nucleic acid sequence encoding at least an active portion of an ErbB-2 polypeptide including an amino acid sequence being at least 60%, at least 69%, at least 70%, at least 73%, at least 77%, at least 80%, at least 85%, at least 95% or more say 100% homologous to SEQ ID NO:6 (portion of variant II), as determined using BlastP software of the National Center of Biotechnology Information (NCBI) using default parameters.

According to a preferred embodiment of this aspect of the present invention the polypeptide encoded by the polynucleotide according to the present invention as described above includes an amino acid sequence being at least 58%, at least 60%, at least 65%, at least 70%, at least 75%, at least 80%, at least 85%, at least 95% or more say 100% identical to SEQ ID NO:6, as determined using BlastP software of the National Center of Biotechnology Information (NCBI) using default parameters.

According to another preferred embodiment of this aspect of the present invention the nucleic acid sequence is as set forth in SEQ ID NO:3, 8 or 15.

Preferably, the polynucleotide according to this aspect of the present invention encodes a polypeptide, which is set forth in SEQ ID NO:4, 6 or 16.

The present invention further features an isolated polynucleotide having a nucleic acid sequence encoding at least an active portion of an ErbB-2 polypeptide including an amino acid sequence being at least 85%, at least 87%, at least 89%, at least 91%, at least 93%, at least 95% or more say 100% homologous to SEQ ID NO:10, 14 or 16 (variant III, IV or V, respectively), as determined using BlastP software of the National Center of Biotechnology Information (NCBI) using default parameters.

According to a preferred embodiment of this aspect of the present invention the polypeptide encoded by the polynucleotide according to the present invention as described above includes an amino acid sequence being at least 85%, at least 87%, at least 89%, at least 91%, at least 93%, at least 95% or more say 100% identical to SEQ ID NO:11, as determined using BlastP software of the National Center of Biotechnology Information (NCBI) using default parameters.

According to another preferred embodiment of this aspect of the present invention the nucleic acid sequence is as set forth in SEQ ID NO:9, 12, 13, or 15.

Preferably, the polynucleotide according to this aspect of the present invention encodes a polypeptide, which is set forth in SEQ ID NO:10, 11, 14 or 16.

The present invention yet further features an isolated polynucleotide including a nucleic acid sequence which encodes an ErbB-2 polypeptide which includes an amino acid sequence at least 63%, at least 65%, at least 70%, at least 73%, at least 75%, at least 80%, at least 83%, at least 85%, at least 87%, at least 89%, at least 91%, at least 93%, at least 95% or more say 100% homologous to SEQ ID NO:26 (variant VI), as determined using the protein Blast search software for short, nearly exact matches of the National Center of Biotechnology Information (NCBI) using default parameters.

According to one preferred embodiment of this aspect of the present invention, the nucleic acid sequence encoding variant VI is as set forth in SEQ ID NO:25.

According to another preferred embodiment of this aspect of the present invention the isolated polynucleotide encodes a polypeptide as set forth in SEQ ID NO:26.

Sequence analysis (available at http://motif.genome.adjpuncovered) of the amino acid sequnece unique to variant VI (amino acid coordinates 341-387 of SEQ ID NO:26) uncovered an inositol phosphate binding domain (e.g., pleckstrin homology domain, PH, SEQ ID NO:28) unique to this variant, suggesting recruitment of this variant to membrane compartments and to signaling pathways which involve molecules such as inositol 1,4,5-trisphosphate/phosphatidylinositol 4,5-bisphosphate, the β?-subunits of heterotrimeric G proteins and protein kinase C known as common ligands for the PH domain [Lemmon (2003) Traffic 4(4):201-13].

The present invention further encompasses various embodiments of the nucleic acid sequences encoding the ErbB-2 variants polypeptides or fragments thereof, as follows:

1. An isolated nucleic acid sequence encoding the polypeptides of ErbB-2 variant I or fragments thereof.

2. An isolated polynucleotide of ErbB-2 variant I as above, wherein the ErbB-2 variant I polypeptide is as set forth in SEQ ID NO: 2 or 5 (portion of var I).

3. An isolated polynucleotide of ErbB-2 variant I, wherein the nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 1.

4 The isolated polynucleotide of ErbB-2 variant I, wherein the nucleic acid sequence is as set forth in SEQ ID NO:1.

5. An isolated nucleic acid sequence encoding the polypeptides of ErbB-2 variant II or fragments thereof.

6. An isolated polynucleotide of ErbB-2 variant II as above, wherein the ErbB-2 variant II polypeptide is as set forth in SEQ ID NO: 4 or 6 (portion of var II).

7. An isolated polynucleotide of ErbB-2 variant II, wherein the nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 3.

8. The isolated polynucleotide of ErbB-2 variant II, wherein the nucleic acid sequence is as set forth in SEQ ID NO: 3.

9. An isolated nucleic acid sequence encoding the polypeptides of ErbB-2 variant III or fragments thereof.

10. An isolated polynucleotide of ErbB-2 variant III as above, wherein the ErbB-2 variant III polypeptide is as set forth in SEQ ID NO: 10 or 11.

11. An isolated polynucleotide of ErbB-2 variant III, wherein the nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 9.

12. The isolated polynucleotide of ErbB-2 variant III, wherein the nucleic acid sequence is as set forth in SEQ ID NO: 9.

13. An isolated nucleic acid sequence encoding the polypeptides of ErbB-2 variant IV or fragments thereof.

14. An isolated polynucleotide of ErbB-2 variant IV as above, wherein the ErbB-2 variant IV polypeptide is as set forth in SEQ ID NO: 14, 5 or 11.

15. An isolated polynucleotide of ErbB-2 variant IV, wherein the nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 13.

16. The isolated polynucleotide of ErbB-2 variant IV, wherein the nucleic acid sequence is as set forth in SEQ ID NO: 13.

17. An isolated nucleic acid sequence encoding the polypeptides of ErbB-2 variant V or fragments thereof.

18. An isolated polynucleotide of ErbB-2 variant V as above, wherein the ErbB-2 variant V polypeptide is as set forth in SEQ ID NO: 16, 6 or 11.

19. An isolated polynucleotide of ErbB-2 variant IV, wherein the nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 15.

20. The isolated polynucleotide of ErbB-2 variant V, wherein the nucleic acid sequence is as set forth in SEQ ID NO: 15.

21 An isolated nucleic acid sequence encoding the polypeptides of ErbB-2 variant VI or fragments thereof.

22. An isolated polynucleotide of ErbB-2 variant VI as above, wherein the ErbB-2 variant VI polypeptide is as set forth in SEQ ID NO: 26 or 28.

23. An isolated polynucleotide of ErbB-2 variant VI, wherein the nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 25.

24. The isolated polynucleotide of ErbB-2 variant VI, wherein the nucleic acid sequence is as set forth in SEQ ID NO: 25.

Thus, the present invention encompasses nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or man induced, either randomly or in a targeted fashion.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the trasgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or nonviral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 5465 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Oligonucleotide Probes and Primers and Methods of Using Same

Oligonucleotides according to the present invention may optionally be used as molecular probes as described herein. Such probes are useful for hybridization assays, and also for NAT assays (as primers, for example).

Typically, detection of a nucleic acid of interest in a biological sample is effected by hybridization-based assays using an oligonucleotide probe.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions. An example of an oligonucleotide probe which can be utilized by the present invention is a single stranded polynucleotide which includes a sequence complementary to the unique sequence region of SEQ ID NO:1 (i.e., complementary to SEQ ID NO. 7). Alternatively, an oligonucleotide probe of the present invention can be designed to hybridize with a nucleic acid sequence encompassed by the bridging sequence between exon 8 and exon 10 (i.e., nucleic acid coordinate 1171 of SEQ ID NO:25) to thereby specifically detect variant VI of the present invention.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of the present invention is of at least 17, at least 18, at least 19, at least 20, at least 22, at least 25, at least 30 or at least 40, bases specifically hybridizable with the ErbB-2 variants of the present invention (non-limiting examples of which are given in SEQ ID NOs. 19-22).

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. U.S. patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8 halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Oligonucleotides of the invention can be utilized with naturally occurring sugar-phosphate backbones as well as modified backbones including phosphorothioates, dithionates, alkyl phosphonates and a-nucleotides and the like. Modified sugar-phosphate backbones are generally taught by Miller, 1988, Ann. Reports Med. Chem. 23:295 and Moran et al., 1987, Nucleic acid molecule. Acids Res., 14:5019. Probes of the invention can be constructed of either ribonucleic acid (RNA) or cleoxyribonucleic acid (DNA), and preferably of DNA.

It will be appreciated that oligonucleotides of the present invention may include further modifications which increase bioavailability, therapeutic efficacy and reduce cytotoxicity. Such modifications are described in Younes (2002) Current Pharmaceutical Design 8:1451-1466.

The isolated polynucleotides of the present invention can optionally be detected (and optionally quantified) by using hybridization assays. Thus, the isolated polynucleotides of the present invention are preferably hybridizable with SEQ ID NO:1, 3, 7, 8, 12, 13, 15, 25 or 27 under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

Hybridization based assays which allow the detection of the ErbB-2 variants of the present invention (i.e., DNA or RNA) in a biological sample rely on the use of oligonucleotides which can be 10, 15, 20, or 30 to 100 nucleotides long, preferably from 10 to 50, and more preferably from 40 to 50 nucleotides.

Hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample (target).

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif] can be attached to the oligonucleotides.

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Although the present invention is not specifically dependent on the use of a label for the detection of a particular nucleic acid sequence, such a label might be beneficial, by increasing the sensitivity of the detection.

Furthermore, it enables automation. Probes can be labeled according to numerous well known methods (Sambrook et al., 1989, supra). Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ATP and polynucleotide kinase, using the Klenow fragment of Pol I of E coli in the presence of radioactive dNTP (i.e. uniformly labeled DNA probe using random oligonucleotide primers in low-melt gels), using the SP6/T7 system to transcribe a DNA segment in the presence of one or more radioactive NTP, and the like.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

As mentioned hereinabove, detection (and optionally quantification) of a nucleic acid of interest in a biological sample may also optionally be effected by nucleic acid amplification technology (NAT)-based assays, which involve nucleic acid amplification technology, such as PCR for example (or variations thereof such as real-time PCR for example).

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

Polymerase chain reaction (PCR) is carried out in accordance with known techniques, as described for example, in U.S. Pat. Nos. 4,683,195; 47683,202; 4,800,159, and 4,965,188 (the disclosures of all three U.S. patents are incorporated herein by reference). In general, PCR involves a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review of PCR techniques, see PCR Protocols, A Guide to Methods and Amplifications, Michael et al. Eds, Acad. Press, 1990.

As used herein, a “primer” defines an digonucleotide which is capable of annealing to a target sequence, thereby creating a double stranded region which can serve as an initiation point for DNA synthesis under suitable conditions.

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

The terminology “amplification pair” refers herein to a pair of oligonucleotides of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligonucleotides are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. Of course, it will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. In general, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity [see Sazani and Kole (2003), supra].

Polymerase chain reaction (PCR)-based methods may be used to identify the presence of mRNA of the ErbB-2 variants of the present invention. For PCR-based methods a pair of oligonucleotides is used, which is specifically hybridizable with the polynucleotide sequences described hereinabove in an opposite orientation so as to direct exponential amplification of a portion thereof (including the hereinabove described sequence alteration) in a nucleic acid amplification reaction. For example, an oligonucleotide pair of primers specifically hybridizable with SEQ ID NO:1 (variant I) is set forth in SEQ ID NOs: 19 and 20, an oligonucleotide pair of primers specifically hybridizable with SEQ ID NO:3 is set forth in SEQ ID NOs: 21 and 22 and an oligonucleotide pair of primers capable of hybridizing with SEQ ID NO:25 is set forth in SEQ ID NOs. 31 and 32.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Hybridization to oligonucleotide arrays may be also used to determine expression of the ErbB-2 variants of the present invention (hybridization itself is described above). Such screening has been undertaken in the BRCA1 gene and in the protease gene of HIV-1 virus [see Hacia et al., (1996) Nat Genet 1996;14(4):441-447; Shoemaker et al., (1996) Nat Genet 1996;14(4):450-456; Kozal et al., (1996) Nat Med 1996;2(7):753-759]. Optionally and preferably, such hybridization is combined with amplification as described herein.

The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for ErbB-related cancers both rapidly and easily.

According to various preferred embodiments of the methods of the present invention, determining the presence and/or level of any specific nucleic or amino acid in a biological sample obtained from, for example, a patient is effected by any one of a variety of methods including, but not limited to, a signal amplification method, a direct detection method and detection of at least one sequence change.

The signal amplification methods according to various preferred embodiments of the present invention may amplify, for example, a DNA molecule or an RNA molecule. Signal amplification methods which might be used as part of the present invention include, but are not limited to PCR, LCR (LAR), Self-Sustained Synthetic Reaction (3SR/NASBA) or a Q-Beta (Qβ) Replicase reaction.

Polymerase Chain Reaction (PCR): The polymerase chain reaction (PCR), as described in U.S. Pat. Nos. 4,683,195 and 4,683,202 to Mullis and Mullis et al, is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] described by Barany, Proc. Natl. Acad. Sci., 88:189 (1991); Barany, PCR Methods and Applic., 1:5 (1991); and Wu and Wallace, Genomics 4:560 (1989) has developed into a well-recognized alternative method of amplifying nucleic acids. In LCR, four oligonucleotides, two adjacent oligonucleotides which unique ly hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes. Segev, PCT Publication No. W09001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR1NASBA): The self-sustained sequence replication reaction (3SR) (Guatelli et al., Proc. Natl. Acad. Sci., 87:1874-1878, 1990), with an erratum at Proc. Natl. Acad. Sci., 87:7797, 1990) is a transcription-based in vitro amplification system (Kwok et at, Proc. Natl. Acad. Sci., 86:1173-1177, 1989) that can exponentially amplify RNA sequences at a uniform temperature. The amplified RNA can then be utilized for mutation detection (Fahy et al., PCR Meth. Appl., 1:25-33, 1991). In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect (Kwok et al., Nucl. Acids Res., 18:999, 1990).

A similar 3′-mismatch strategy is used with greater effect to prevent ligation in the LCR (Barany, PCR Meth. Applic., 1:5, 1991). Any mismatch effectively blocks the action of thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), described by Urdea et al., Gene 61:253-264 (1987), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perlman and Butow, Science 246:1106, 1989), but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras germs (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al, Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and net be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base lair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for the mutation or mutations in any of the genes listed above, such as, for example, the reduced folate carrier (RFC) gene, in tumor cells or in cells derived from a cancer patient is effected by a single strand conformational polymorphism (SSCP) technique, such as cDNA-SSCP or genomic DNA-SSCP. However, alternative methods can be employed, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Methods of Treatment

As mentioned hereinabove the secreted ErbB-2 variants of the present invention and compositions derived therefrom (i.e., peptides, oligonucleotides) can be used to treat ErbB-related cancer.

Thus, according to an additional aspect of the present invention there is provided a method of treating ErbB-related cancer in a subject.

As used herein the phrase “ErbB-related cancer” refers to a malignant or benign tumor which is dependent on expression or activity of at least one ErbB protein (i.e., ErbB-1, ErbB-2, ErbB-3 and ErbB-4) for its abnormal growth [Agus Cancer cell (2002), 2:93-95; Mendoxa Cancer Res., (2002) 62:5485-5488]. Abnormal growth rate is a rate of growth which is in excess of that required for normal homeostasis and is in excess of that for normal tissues of the same origin.

Examples of ErbB-related cancer types include, but are not limited to bladder cancer, breast cancer, testis cancer, cancers of the central nervous system (e.g., head and neck), sarcomas, prostate cancer, pancreatic cancer, ovarian cancer, lung cancer, gastric cancer, esophageal cancer, endometrial cancer, colorectal cancer, salivary gland cancer, renal cancer, oral cancer and cervical cancer.

The subject according to the present invention is a mammal, preferably a human which has at least one type of the tumors described hereinabove.

As used herein the term “treating” refers to preventing, curing, reversing, attenuating, alleviating, minimizing, suppressing or halting the deleterious effects of an ErbB-related cancer.

The method is effected by specifically upregulating the amount (optionally expression) in the subject of at least one of the polypeptides of the present invention. As described hereinabove upregulation of the polypeptides of the present invention or active portions thereof can result in inactivation of the ErbB signaling cascade such as via the formation of inactive dimers (i.e., a dominant negative effect), to thereby treat the ErbB-related cancer in the subject.

Upregulating expression of the ErbB-2 variants of the present invention may be effected via the administration of at least one of the exogenous polynucleotide sequences of the present invention, ligated into a nucleic acid expression construct designed for expression of coding sequences in eukaryotic cells (e.g., mammalian cells), as described above (see “nucleic acid sequences according to the present invention” section). Accordingly, the exogenous polynucleotide sequence may be a DNA or RNA sequence encoding the variants (variant I-VI) of the present invention or active portions thereof (for example, SEQ ID NOs: 7, 8, 12 or 27).

It will be appreciated that the nucleic acid construct can be administered to the individual employing any suitable mode of administration, described hereinbelow (i.e., in-vivo gene therapy). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy). Nucleic acid constructs are described in greater detail above.

It will be appreciated that the present methodology may also be effected by specifically upregulating the expression of the variants of the present invention endogenously in the subject. Agents for upregulating endogenous expression of specific splice variants of a given gene include antisense oligonucleotides, which are directed at splice sites of interest, thereby altering the splicing pattern of the gene. This approach has been successfully used for shifting the balance of expression of the two isoforms of Bcl-x [Taylor (1999) Nat. Biotechnol. 17:1097-1100; and Mercatante (2001) J. Biol. Chem. 276:16411-16417]; IL-5R [Karras (2000) Mol. Pharmacol. 58:380-387]; and c-myc [Giles (1999) Antisense Acid Drug Dev. 9:213-220].

For example, interleukin 5 and its receptor play a critical role as regulators of hematopoiesis and as mediators in some inflammatory diseases such as allergy and asthma. Two alternatively spliced isoforms are generated from the IL-5R gene, which include (i.e., long form) or exclude (i.e., short form) exon 9. The long form encodes for the intact membrane-bound receptor, while the shorter form encodes for a secreted soluble non-functional receptor. Using 2′-O-MOE-oligonucleotides specific to regions of exon 9, Karras and co-workers (supra) were able to significantly decrease the expression of the wild type receptor and increase the expression of the shorter isoforms. Design and synthesis of oligonucleotides which can be used according to the present invention are described hereinbelow and by Sazani and Kole (2003) Progress in Moleclular and Subcellular Biology 31:217-239.

It will be appreciated that the present invention also envisages treatment of cancers in which the ErbB variants of the present invention play a proactive role in inducing cancer formation and/or progression. Such cancers include for example, cases where variants of ErbB-2 of the present invention envoke direct signaling (i.e., by acting as ligands), cross signaling (i.e., signaling which involves components of the ErbB signaling network and components of other signaling cascades in a single signaling pathway) or reverse signaling via the binding thereof to membrane anchored growth factors, such as neuregulin [Murai J Cell Sci. 2003 Jul. 15; 116(Pt 14):2823-32; Sporri Cytokine Growth Factor Rev. 2001 March;12(1):27-32; Kliewer Science. 1999 Apr. 30;284(5415):757-60] and thus directly induce cancer formation.

It will be appreciated that in such cancers, treatment is preferably effected by agents which are capable of specifically downregulating expression (or activity) of at least one of the polypeptide variants of the present invention.

Down regulating the expression of the ErbB-2 polypeptide variants of the present invention may be achieved using oligonucleotide agents such as those described in greater detail below.

SiRNA molecules—Small interfering RNA (siRNA) molecules can be used to down-regulate expression of the ErbB-2 variants of the present invention. RNA interference is a two step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates fiat each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. Target sites are selected from the unique nucleotide sequences of each of the polynucleotides of the present invention, such that each polynucleotide is specifically down regulated. Thus, to specifically down regulate variant I the siRNA olignonucleotide is directed at SEQ ID NO:7. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

DNAzyme molecules—Another agent capable of downregulating expression of the polypeptides of the present invention is a DNAzyme molecule capable of specifically cleaving an mRNA transcript or DNA sequence of the polynucleotides of the present invention. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Target sites for DNAzymes are selected from the unique nucleotide sequences of each of the polynucleotides of the present invention, such that each polynucleotide is specifically down regulated. Thus, to specifically down regulate variant II the siRNA olignonucleotide is directed at SEQ ID NO:8.

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org. In another application, DNAzymes complementary to bcr-ab1 oncogenes were successful in inhibiting the oncogenes expression in leukemia cells, and lessening relapse rates in autologous bone marrow transplant in cases of CML and ALL.

Antisense molecules—Downregulation of the polynucleotides of the present invention can also be effected by using an antisense polynucleotide capable of specifically hybridizing with an mRNA transcript encoding the polypeptide variants of the present invention.

Design of antisense molecules which can be used to efficiently down-regulate expression of the polypeptides of the present invention must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al, Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Target sites for antisense molecules are selected from the unique nucleotide sequences of each of the polynucleotides of the present invention, such that each polynucleotide is specifically down regulated. Thus, to specifically down regulate variant I the siRNA olignonucleotide is directed at SEQ ID NO:7.

Ribozymes—Another agent capable of downregulating expression of the polypeptides of the present invention is a ribozyme molecule capable of specifically cleaving an mRNA transcript encoding the polypeptide variants of the present invention. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In therapeutics area, ribozymes have been exploited to target viral RNAs in infectious diseases, dominant oncogenes in cancers and specific somatic mutations in genetic disorders [Welch et al, Clin Diagn Virol. 10:163-71 (1998)]. Most notably, several ribozyme gene therapy protocols for HIV patients are already in Phase I trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Alternatively, down regulation of the polypeptide variants of the present invention may be achieved at the polypeptide level using downregulating agents such as antibodies or antibody fragments capabale of specifically binding the polypeptides of the present invention and inhibiting the activity thereof (i.e., neutralizing antibodies). Such antibodies can be directed for example, to the heterodimerizing domain on the variant, or to a putative ligand binding domain. Further description of antibodies and methods of generating same is provided below (see “Antibodies and Immunoassays” section).

Diagnostic Methods and Assays

In addition to therapeutic advances pioneered by the present invention, the novel variants of ErbB-2 of the present invention may be also employed in diagnostic applications. As used herein the term “diagnosing” refers to classifying a disease or a symptom as an ErbB-related cancer, determining a severity of the ErbB-related cancer, monitoring disease progression, forecasting an outcome of a disease and/or prospects of recovery.

Without being bound by theory, since at least certain polypeptides of the present invention are secreted variants of the ErbB-2 protein it is likely that such circulating polypeptides envoke signaling (i.e., by acting as ligands), trans signaling (i.e., signaling which involves components of the ErbB signaling network and components of other signaling cascades in a single signaling pathway, such as the signaling reported for soluble IL-6R, Kallen Biochim Biophys Acta. 2002 Nov. 11; 1592(3):323-43) or reverse signaling, via binding to membrane anchored growth factors, such as neuregulin [Murai J Cell Sci. 2003 Jul. 15;116(Pt 14):2823-32; Sporri Cytokine Growth Factor Rev. 2001 March;12(1):27-32; Kliewer Science. 1999 Apr. 30;284(5415):757-60]. Alternatively, the soluble polypeptides of the present invention may have a structural stabilization effect of the ligand, protect it from degradation and/or prolong its half-life, consistent with a role of carrier proteins. Regardless of the mechanism of action, such soluble variants may potentiate receptor activity in-vivo, and as such be used in diagnostic applications.

Thus, according to yet an additional aspect of the present invention there is provided a method of diagnosing predisposition to, or presence of ErbB-2 related cancer in a subject. However, it should be noted that diagnostic assays, kits and methods described herein are not limited to detection of a protein, peptide, polypeptide or polypeptide fragment, but instead may also optionally encompass use of oligonucleotide probes and/or hybridization and/or NAT type methods, kits and assays.

The term “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The method is effected by determining a level of a polynucleotide or a polypeptide of the present invention in a biological sample obtained from the subject, wherein the level determined can be correlated with predisposition to, or presence or absence of the ErbB-related cancer.

As used herein, the term “level” refers to expression levels of RNA and/or protein or to DNA copy number of the ErbB-2 variants of the present invention.

A level correlatable with predisposition to, or presence or absence of ErbB-related cancer can be a level of an ErbB-2 variant of the present invention in a cancerous sample which is different different (i.e., increased or descreased) from the level of the same variant in a normal healthy sample obtained from a similar tissue or cellular origin.

As used herein “a biological sample” refers to a sample of tissue or fluid isolated from a subject, including but not limited to, for example, plasma, serum, spinal fluid, lymph fluid, the external sections of the skin, respiratory, intestinal, and genitourinary tracts, tears, saliva, milk, blood cells, tumors, neuronal tissue, organs, and also samples of in vivo cell culture constituents. For example, tissue would optionally and preferably include breast or other tissues. A fluid sample would optionally and preferably include blood (optionally including whole blood and/or blood fractions), or urine, for example.

Numerous well known tissue or fluid collection methods can be utilized to collect the biological sample from the subject in order to determine the level of DNA, RNA and/or polypeptide of the ErbB-2 variants of the present invention in the subject.

Examples include, but are not limited to, lavage, fine needle biopsy, needle biopsy, core needle biopsy and surgical biopsy.

Regardless of the procedure employed, once a biopsy is obtained the level of the ErbB-2 variants of the present invention can be determined and a diagnosis can thus be made.

Determining a level of the ErbB-2 variants of the present invention can be effected using various biochemical and molecular approaches used in the art for determining gene amplification, and/or level of gene expression.

Determining the level of the ErbB-2 variants of the present invention in normal tissues of the same origin is preferably effected along side to detect an elevated expression and/or amplification. Additionally or alternatively, determining the level of wild-type ErbB-2 (or at least the extracellular portion of the ErbB-2 gene product, such as sp185-Her2 from Bender MedSystems GmbH (Austria)) is preferably effected along side.

The term “marker” in the context of the present invention refers to a nucleic acid fragment, a peptide, or a polypeptide, which is differentially present in a sample taken from patients having ErbB-related cancer, for example breast cancer, as compared to a comparable sample taken from subjects who do not have said cancer.

The phrase “differentially present” refers to differences in the quantity of a marker present in a sample taken from patients having ErbB-related cancer, for example breast cancer, as compared to a comparable sample taken from patients who do not have said cancer. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present.

A “test amount” of a marker refers to an amount of a marker present in a sample being tested. A test amount can be either in absolute amount (e.g., microgram/ml) or a relative amount (e.g., relative intensity of signals), for example relative to a control.

A “diagnostic amount” of a marker refers to an amount of a marker in a subject's sample that is consistent with a diagnosis of ErbB-related cancer, for example breast cancer. A diagnostic amount can be either in absolute amount (e.g., microgram/ml) or a relative amount (e.g., relative intensity of signals), for example relative to a control.

A “control amount” of a marker can be any amount or a range of amounts to be compared against a test amount of a marker. For example, a control amount of a marker can be the amount of a marker in an ErbB-related cancer patient, for example in a breast cancer patient, or a person without said cancer. A control amount can be either in absolute amount (e.g., microgram/ml) or a relative amount (e.g., relative intensity of signals).

The presence of the ErbB-2 variants of the present invention may also be detected at the protein level. Numerous protein detection assays are known in the art, examples include, but are not limited to, chromatography, electrophoresis, immunodetection assays such as ELISA and western blot analysis, immunohistochemistry and the like, which may be effected using antibodies specific to the ErbB-2 variants of the present invention.

Diagnosing and Monitoring Breast Cancer Using the Markers of the Present Invention

As is illustrated in Example 2 of the Examples section the new ErbB-2 splice variants detectable by the sequences as depicted in SEQ ID NO: 50 or 53 (for example SEQ ID Nos: 1, 13, 3, 15 or the unique sequences as depicted in SEQ ID NO: 7, 8) are differentially expressed in breast cancer, and thus can be used for detection of, as well as for monitoring of progression and/or determining efficacy of treatment of breast cancer.

Thus, the present invention features a biomarker for detecting breast cancer, comprising ErbB-2 splice variants sequence, detectable by the sequences as depicted in SEQ ID NO: 50 or 53 (for example SEQ ID Nos: 1,13, 3, 15, or the unique sequences as depicted in SEQ ID NO: 7, 8), or a fragment thereof.

According to a further aspect of the present invention there is provided a biomarker for detecting a predisposition to, monitoring of progression of, or detemining the efficacy of treatment of, breast cancer, comprising one or more of the ErbB-2 variant sequences or a fragment thereof. According to the present invention, the ErbB-2 variants detectable by the sequences as depicted in SEQ ID NO: 50 or 53 (for example SEQ ID Nos: 1, 13, 3, or 15, or the unique sequences as depicted in SEQ ID NO: 7, 8) are a non-limiting example of a marker for diagnosing breast cancer. Although optionally any method may be used to detected overexpression and/or differential expression of this marker, preferably a NAT-based technology is used. Therefore, optionally and preferably, any nucleic acid molecule capable of selectively hybridizing to the ErbB-2 variants as previously defined is also encompassed within the present invention. Primer pairs are also optionally and preferably encompassed within the present invention; for example, for the above experiment, the following primer pairs were used as a non-limiting illustrative example only of a suitable primer pairs: ErbB-2-long variant—forward primer: TGTGAGGGACACAGGCAAAGT (SEQ ID NO: 48); and ErbB-2-long variant—Reverse primer: CCCACCATCCCCAGTTAAGAA (SEQ ID NO: 49); ErbB-2-short variant—forward primer: CAGCGTTCTTGGACTTGTGC (SEQ ID NO: 51); and ErbB-2-short variant—Reverse primer: CCAGCTAGAGAAGCCATGCC (SEQ ID NO: 52).

The present invention also preferably encompasses any amplicon obtained through the use of any suitable primer pair; for example, for the above experiment, the following amplicons were obtained as a non-limiting illustrative example only of a suitable amplicons: ErbB-2-long variant—amplicon: TGTGAGGGACACAGGCAAAGTTCAATTCCTTGGAAGTCAAGGGAGACTGA GAAGAGTACAGCTGCAGCACTGAGGGAGTGATGAATTCTTAACTGGGGAT GGTGGG (SEQ ID NO 50); ErbB-2-short variant—amplicon: CAGCGTTCTTGGACTTGTGCAGACTGCCCGTCTCTGTGCACCCTTCTTGAC TCAGCACAGCTCTGGCTGGCTTGGCCTCTTGGCATGGCTTCTCTAGCTGG (SEQ ID NO 53).

According to other preferred embodiments of the present invention, the ErbB-2 variants detectable by the sequences as depicted in SEQ ID NO: 50 or 53 or a fragment thereof comprises a biomarker for detecting breast cancer. Optionally and more preferably, ErbB-2 splice variants, as depicted in SEQ ID NO: 1, 13, 3, or 15, or the unique sequences as depicted in SEQ ID NO: 7, 8, or a fragment thereof comprise a biomarker for detecting breast cancer. Also optionally and more preferably, any suitable method may be used for detecting a fragment such as fragment of ErbB-2 variant of the present invention, such as for example SEQ ID NO: 50 or 53. Most preferably, NAT-based technology used, such as any nucleic acid molecule capable of specifically hybridizing with the fragment. Optionally and most preferably, a primer pair is used for obtaining the fragment.

According to still other preferred embodiments, the present invention optionally and preferably encompasses any amino acid sequence or fragment thereof encoded by a nucleic acid sequence corresponding to ErbB-2 variants detectable by the sequences as depicted in SEQ ID NO: 50 or 53, as described above, including but not limited to amino acid sequences as depicted in SEQ ID NOs:2, 14, 4, 16. The present invention also optionally and preferably encompasses the unique amino acid tails of ErbB-2 long and short variants, including but not limited to the unique tails as depicted in SEQ ID NOs: 5 or 6. Any oligopeptide or peptide relating to such an amino acid sequence or fragment thereof may optionally also (additionally or alternatively) be used as a biomarker for detecting breast cancer.

The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to ErbB-2 variants detectable by the sequences as depicted in SEQ ID NO: 50 or 53 as described above, optionally for any application.

According to a further aspect of the present invention there is provided use for an assay for detecting or monitoring progression of, or determining efficacy of treatment of breast cancer, comprising: an assay detecting overexpression of ErbB-2 variants detectable by the sequences as depicted in SEQ ID NO: 50 or 53 as described above, or a fragment thereof.

The assay can comprise, for example, a NAT-based technology.

According to a further aspect of the present invention there is provided a method for detecting or monitoring progression of, or determining efficacy of treatment of breast cancer, comprising: detecting overexpression of the ErbB-2 variants detectable by the sequences as depicted in SEQ ID NO: 50 or 53 or a fragment thereof.

The method can be performed, for example, by using NAT-based technology.

Antibodies and Immunoassays

Preferably, an antibody of this aspect of the present invention specifically binds at least one epitope of the polypeptide variants of the present invention. As used herein, the term “epitope” refers to any antigenic determinant on an antigen to which the paratope of an antibody binds.

For example, an antibody or an antibody fragment of the present invention can be generated to specifically bind to an amino acid sequence (epitope) present in any of the amino acid sequences below (as well as to any amino acid sequence described above):

1. An isolated polypeptide of ErbB-2 variant I (SEQ ID NO: 2), comprising a first amino acid sequence being at least 90% homologous to amino acids 1-648 of wild type ErbB-2 (GenBank Accession No: gi: AAA75493), which also encompasses amino acids 1-648 of the sequence as set forth in SEQ ID NO:2, and a second amino acid sequence being at least about 70%, optionally at least about 80%, preferably at least about 85%, more preferably at least about 90% and most preferably at least about 95% homologous to a polypeptide having the sequence RLAWTP GCTLHCPSLPHWMLGGHCCREGTP (SEQ ID NO: 5), wherein the first and the second amino acid sequences are contiguous and in a sequential order.

2. An isolated polypeptide of a tail of ErbB-2 variant I (SEQ ID NO: 2), comprising a polypeptide having the sequence RLAWTPGCTLHCPSLPHWMLGGHCCREGTP (SEQ ID NO: 5).

3. A unique edge portion of SEQ ID NO: 2, comprising a polypeptide being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 5).

4. A bridge portion of SEQ ID NO:2, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AR, having a structure as follows (numbering according to SEQ ID NO:2): a sequence starting from any of amino acid number 648-x to 648; and ending at any of amino acid number ending at any of amino acid numbers 649+((n−2)−x), in which x varies from 0 to n−2, with the proviso that the value ((n−2)−x) is not allowed to be larger than 29.

The bridge portion of SEQ ID NO:2 above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: ARLA, RARL, QRAR.

5. An isolated polypeptide of ErbB-2 variant II (SEQ ID NO: 4), comprising a first amino acid sequence being at least 90% homologous to amino acids 1-504 of wild type ErbB-2 (GenBank Accession No: gi: AAA75493), which also encompasses amino acids 1-504 of the sequence as set forth in SEQ ID NO: 4, and a second amino acid sequence being at least about 70%, optionally at least about 80%, preferably at least about 85%, more preferably at least about 90% and most preferably at least about 95% homologous to a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6), wherein the first and the second amino acid sequences are contiguous and in a sequential order.

6. An isolated polypeptide of a tail of ErbB-2 variant II (SEQ ID NO: 4), comprising a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6).

7. A unique edge portion of SEQ ID NO: 4, comprising a polypeptide being at least 70%, optionally at least about 80%, preferably at least about 85%, more preferably at least about 90% and most preferably at least about 95% homologous to the sequence GKTGSPVCALPICQHTAVPRGPWQQRSWTCADCPSLCTLLDSAQLWLAWPL GMASLAGSYLPWHPSLPLCF (SEQ ID NO: 6).

8. A bridge portion of SEQ ID NO:4, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise CG, having a structure as follows (numbering according to SEQ ID NO:4): a sequence starting from any of amino acid number 504-x to 504; and ending at any of amino acid number ending at any of amino acid numbers 505+((n−2)−x), in which x varies from 0 to n−2, with the proviso that the value ((n−2)−x) is not allowed to be larger than 70.

The bridge portion of SEQ ID NO: 4 above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: CGKT, ECGK, DECG.

9. An isolated polypeptide of ErbB-2 variant III (SEQ ID NO: 10), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 10, an amino acid sequence being at least about 70%, optionally at least about 80%, preferably at least about 85%, more preferably at least about 90% and most preferably at least about 95% homologous to a polypeptide having the sequence VSLCQQAGVQWYDLGSLQPLPPGFKQFSCLSLLSSWDYR (SEQ ID NO: 11), and a second amino acid sequence being at least 90% homologous to amino acids 384-1255 of wild type ErbB-2 (GenBank Accession No: gi: AAA75493), which also encompasses amino acids 423-1294 of the sequence as set forth in SEQ ID NO: 10, wherein the first amino acid is contiguous to the bridging polypeptide and the second amino acid sequence is contiguous to the bridging polypeptide, and wherein the first amino acid, the bridging polypeptide and the second amino acid sequence are in a sequential order.

10. An isolated polypeptide encoding for an edge portion of ErbB-2 variant III (SEQ ID NO: 10), comprising a polypeptide having the sequence VSLCQQAGVQWYDLGSLQPLPPGFKQFSCLSLLSSWDYR (SEQ ID NO: 11).

11. A unique edge portion of SEQ ID NO: 10, comprising a polypeptide being at least 70%, optionally at least about 80%, preferably at least about 85%, more preferably at bast about 90% and most preferably at least about 95% homologous to the sequence VSLCQQAGVQWYDLGSLQPLPPGFKQFSCLSLLSSWDYR (SEQ ID NO: 11).

12. An bridge portion of SEQ ID NO: 10, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise GV, having a structure as follows (numbering according to SEQ ID NO:10): a sequence starting from any of amino acid number 383-x to 383; and ending at any of amino acid numbers 384+((n−2)−x), in which x varies from 0 to n−2 in which x varies from 0 to n−2.

The bridge portion above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: GVSL, DGVS, FDGV.

13. A second bridge portion of SEQ ID NO: 10, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RD, having a structure as follows (numbering according to SEQ ID NO:10): a sequence starting from any of amino acid number 421-x to 421; and ending at any of amino acid numbers 422+((n−2)−x), in which x varies from 0 to n−2.

The bridge portion of SEQ ID 10 above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: DYRD, YRDP, RDPA.

14. An isolated polypeptide of ErbB-2 variant IV (SEQ ID NO: 14), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 14, an amino acid sequence being a least about 70%, optionally at least about 80%, preferably at least about 85%, more preferably at least about 90% and most preferably at least about 95% homologous to a polypeptide having the sequence VSLCQQAGVQWYDLGSLQPLPPGFKQFSCLSLLSSWDYR (SEQ ID NO: 11), a second amino acid sequence being at least 90% homologous to amino acids 384-648 of wild type ErbB-2 (GenBank Accession No: gi: AAA75493), which also encompasses amino acids 423-687 of the sequence as set forth in SEQ ID NO: 14, followed by an amino acid sequence tail being at least about 70%, optionally at least about 80%, preferably at least about 85%, more preferably at least about 90% and most preferably at least about 95% homologous to a polypeptide having the sequence RLAWTPGCTLHCPSLPHWMLGGHCCREGTP (SEQ ID NO: 5), wherein the first amino acid is contiguous to the bridging polypeptide and the second amino acid sequence is contiguous to the bridging polypeptide, and wherein the first amino acid, the bridging polypeptide, the second amino acid and the amino acid sequence tail are in a sequential order.

15. An isolated polypeptide of ErbB-2 variant V (SEQ ID NO: 16), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 16, an amino acid sequence being at least about 70%, optionally at least about 80%, preferably at least about 85%, more preferably at least about 90% and most preferably at least about 95% homologous to a polypeptide having the sequence VSLCQQAGVQWYDLGSLQPLPPGFKQFSCLSLLSSWDYR (SEQ ID NO: 11), a second amino acid sequence being at least 90% homologous to amino acids 384-804 of wild type ErbB-2 (GenBank Accession No: gi: AAA75493), which also encompasses amino acids 423-543 of the sequence as set forth in SEQ ID NO: 16, followed by an amino acid sequence tail being at least about 70%, optionally at least about 80%, preferably at least about 85%, more preferably at least about 90% and most preferably at least about 95% homologous to a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6), wherein the first amino acid is contiguous to the bridging polypeptide and the second amino acid sequence is contiguous to the bridging polypeptide, and wherein the first amino acid, the bridging polypeptide, the second amino acid and the amino acid sequence tail are in a sequential order.

16. An isolated polypeptide of ErbB-2 variant VI (SEQ ID NO: 26), comprising a first amino acid sequence being at least 90% homologous to amino acids 1-340 of wild type ErbB-2 (GenBank Accession No: gi: AAA75493), which also encompasses amino acids 1-340 of the sequence as set forth in SEQ ID NO:26, and a second amino acid sequence being at least about 70%, optionally at least about 80%, preferably at least about 85%, more preferably at least about 90% and most preferably at least about 95% homologous to a polypeptide having the sequence GTQPPTLPRSSQSSSKCLRLWKRSQVTYTSQHGRTACLTSASSRTCK (SEQ ID NO: 28), wherein the first and the second amino acid sequences are contiguous and in a sequential order.

17. An isolated polypeptide of a tail of ErbB-2 variant VI (SEQ ID NO: 26), comprising a polypeptide having the sequence GTQPPTLPRSSQSSSKCLRLWKRSQVTYTSQHGRTACLTSASSRTCK (SEQ ID NO: 28).

18. A unique edge portion of SEQ ID NO: 26, comprising a polypeptide being at least 70%, optionally at least about 80%, preferably at least about 85%, more preferably at least about 90% and most preferably at least about 95% homologous to the sequence GTQPPTLPRSSQSSSKCLRLWKRSQVTYTSQHGRTACLTSASSRTCK (SEQ ID NO: 28).

19. A bridge portion of SEQ ID NO:26, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RG, having a structure as follows (numbering according to SEQ ID NO:26): a sequence starting from any of amino acid number 340-x to 340; and ending at any of amino acid numbers 341+((n−2)−x), in which x varies from 0 to n−2, with the proviso that the value ((n−2)−x) is not allowed to be larger than 46.

The bridge portion of SEQ ID NO:26 above may optionally and preferably comprise a polypeptide being at least 70%, optionally at least about 80%, preferably at least about 85%, more preferably at least about 90% and most preferably at least about 95% homologous to at least one sequence described above.

Similarly, the bridge portion may optionally be relatively short, such as from about 4 to about 9 amino acids in length. For four amino acids, the first bridge portion would comprise the following peptides: CARG, ARGT, RGTQ, whereby the antibody is capable of distinguishing the ErbB-2 variant protein from the wild type ErbB-2 protein corresponding to accession number: AAA75493.

According to another preferred embodiment of this aspect of the present invention the active portion of the polypeptide is selected to include an epitope which flanks a sequence region common to full-length ErbB-2 as well as to variant I and the unique sequence region of variant I. Measures are taken that such a sequence is uniquely identified by antibodies raised thereagainst. Thus, for example, the active portion of the polypeptide includes amino acid coordinates 645-652 of SEQ ID NO:2, which is encoded by nucleotide coordinates 2109-2130 of SEQ ID NO: 1 (see above for a description of nucleic acid sequences according to the present invention).

According to another preferred embodiment of this aspect of the present invention the active portion of the polypeptide is selected to include an epitope which flanks a sequence region common to full-length ErbB-2 as well as to variant II and the unique sequence region of variant II. Measures are taken that such a sequence is uniquely identified by antibodies raised thereagainst. Thus, for example, the active portion of the polypeptide includes amino acid coordinates 501-508 of SEQ ID NO:4, which is encoded by nucleotide coordinates 1652-1673 of SEQ ID NO:3.

According to another preferred embodiment of this aspect of the present invention the active portion of the polypeptide is selected to include an epitope which flanks a sequence region common to full-length ErbB-2 as well as to variant III, IV or V and the unique sequence region of variants III, IV and V. Measures are taken that such a sequence is uniquely identified by antibodies raised thereagainst. Thus, for example, the active portion of the polypeptide includes amino acid coordinates 381-388 of SEQ ID NO:9.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)₂, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from nonhuman immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and coworkers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al, Monoclonal Antibodies and Cancer Therapy, Alan R. Liss, p. 77 (1985) and Boerner et al, J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al, Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Antibodies of the present invention may optionally be used in diagnostic assays, to detect the presence of a protein or peptide marker, as described above; antibodies may also optionally be used as a therapeutic treatment, alone or in combination with other such treatments. Illustrative, non-limiting examples of suitable uses of antibodies for treatments are given in greater detail below.

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

These immunoassays may optionally be used for a variety of purposes, including but not limited to, prognosis of the course or a disease and/or pathological condition, prediction of susceptibility to such a disease or pathological condition, screening, early diagnosis, therapy selection and treatment monitoring (optionally including staging of the disease and/or pathological condition).

To prepare an antibody that specifically binds to a marker, purified protein markers can be used. Antibodies that specifically bind to a protein marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989); and Example 3 of the Examples section).

After the antibody is provided, a marker can be detected and/or quantified using any of a number of well recognized immunological binding assays (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. For a review of the general immunoassays, see also, Methods in Cell Biology: Antibodies in Cell Biology, volume 37 (Asai, ed. 1993); Basic and Clinical Immunology (Stites & Terr, eds., 7th ed. 1991).

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a substrate as described above. The sample is preferably a biological fluid sample taken from a subject. Examples of biological fluid samples include blood, serum, urine, prostatic fluid, seminal fluid, semen, seminal plasma and lung tissue (e.g., epithelial tissue, including extracts thereof). In a preferred embodiment, the biological fluid comprises seminal plasma. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay techniques are well-known in the art, and a general overview of the applicable technology can be found in the references described above and incorporated by reference. Immunological detection methods are fully explained in, for example, “Using Antibodies: A Laboratory Manual” (Ed Harlow, David Lane eds., Cold Spring Harbor Laboratory Press (1999)) and those familiar with the art will be capable of implementing the various techniques summarized hereinbelow as part of the present invention. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, A labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a calorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. As noted above, the test amount of marker need not be measured in absolute units, as long as the unit of measurement can be compared to a control amount.

Antibodies may also optionally be used for therapeutic applications (also as described in greater detail below). As such, the antibodies may optionally be prepared as previously described (for example with regard to preparing humanized antibodies and/or fragments). Therapeutic antibodies may optionally comprise a functional moiety (for example having a reactive molecule covalently bound thereof), which may for example be a cytotoxic moiety or agent (for example to kill cancer cells) and/or a diagnostic moiety (for example a fluorescent dye for detecting a variant according to the present invention, for example in a cancer cell, whether in vitro or in vivo).

For any of the above optional, exemplary, non-limiting applications, antibodies are preferably used which specifically interact with the polypeptides of the present invention and not with wild type ErbB-2 protein or other isoforms thereof, for example. Such antibodies are directed, for example, to the unique sequence portions of the polypeptide variants of the present invention (e.g., SEQ ID NOs: 5, 6, 11 or 28) or to unique sequences, which bridge the ErbB-2 common portion and the unique sequence regions as described above.

The reagents described hereinabove can also be included in diagnostic or therapeutic kits. For example a kit for diagnosing predisposition to, or presence of ErbB-related cancer in a subject can include an antibody directed at the unique amino acid sequence of variant I-VI (for example SEQ ID NOs: 5, 6, 11 or 28, and/or any of the above bridge sequences and/or previously described exemplary epitopes) in a one container and a solid phase for attaching multiple biological samples packaged in a second container with appropriate buffers and preservatives and used for diagnosis.

Pharmaceutical Compositions and Delivery Thereof

The present invention features a pharmaceutical composition comprising a therapeutically effective amount of a therapeutic agent according to the present invention, which is preferably an ErbB-2 variant as described herein, optionally and preferably B2S or B2L as described herein. Optionally and alternatively, the therapeutic agent could be an antibody or an oligonucleotide that specifically recognizes and binds to B2S or B2L, but not to the full length (oncogenic) ErbB-2 protein.

Optionally and preferably, the ErbB-2 variant features at least an active portion of an ErbB-2 polypeptide including an amino acid sequence being at least 71% homologous to SEQ ID NO:5, as determined using the BlastP software of the National Center of Biotechnology Information (NCBI) using default parameters. More preferably, the polypeptide is as set forth in SEQ ID NO:2, 5 or 14; most preferably, the active portion of the polypeptide is as set forth in SEQ ID NO:5. Also optionally and most preferably, the active portion of the polypeptide is encoded by nucleotide coordinates 2097-2320 of SEQ ID NO:1.

Alternatively, the pharmaceutical composition of the present invention includes a therapeutically effective amount of at least an active portion of an ErbB-2 polypeptide including an amino acid sequence being at least 69% homologous to SEQ ID NO:6, as determined using the BlastP software of the National Center of Biotechnology Information (NCBI) using default parameters. Preferably, the polypeptide is as set forth in SEQ ID NO:4, 6 or 16. More preferably, the active portion of the polypeptide is as set forth in SEQ ID NO:6. Most preferably, the active portion of the polypeptide is encoded by nucleotide coordinates 1664-1944 of SEQ ID NO:3.

The pharmaceutical composition according to the present invention is preferably used for the treatment of cancer, preferably including but not limited to, breast, colon, rectal and colorectal cancer.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder. “Mammal” for purposes of treatment refers to any animal classified as a mammal, including humans, domestic and farm animals, and zoo, sports, or pet animals, such as dogs, horses, cats, cows, etc. Preferably, the mammal is human.

A “disorder” is any condition that would benefit from treatment with the agent according to the present invention. This includes chronic and acute disorders or diseases including those pathological conditions which predispose the mammal to the disorder in question. Non-limiting examples of disorders to be treated herein include benign and malignant tumors; leukemias and lymphoid malignancies; neuronal, glial, astrocytal, hypothalamic and other glandular, macrophagal, epithelial, stromal and blastocoelic disorders; and inflammatory, angiogenic and immunologic disorders.

The term “therapeutically effective amount” refers to an amount of agent according to the present invention that is effective to treat a disease or disorder in a mammal. In the case of cancer, the therapeutically effective amount of the agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the agent may prevent growth and/or kill existing cancer cells, it may be cytostatic and/or cytotoxic. For cancer therapy, efficacy can, for example, be measured by assessing the time to disease progression (TTP) and/or determining the response rate (RR).

The terms “cancer” and “cancerous” refer to or describe the physiological condition in mammals that is typically characterized by unregulated cell growth. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

The present invention also preferably features a method of treating cancer that expresses ErbB2 selected from the group consisting of carcinoma, lymphoma, blastoma, medulloblastoma, retinoblastoma, sarcoma, liposarcoma, synovial cell sarcoma, neuroendocrine tumor, carcinoid tumor, gastrinoma, islet cell cancer, mesothelioma, schwannoma, acoustic neuroma, meningioma, adenocarcinoma, melanoma, leukemia, lymphoid malignancy, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastror stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, a tumor of the biliary tract, and head and neck cancer, comprising administering a therapeutically effective amount of an ErbB-2 variant according to the present invention to a patient.

The present invention also preferably features an article of manufacture is provided which comprises a container and a composition contained therein, wherein the composition comprises ErbB-2 variant according to the present invention, which optionally and preferably blocks ligand activation of an ErbB receptor, and further comprises a package insert indicating that the composition can be used to treat a cancer, optionally and more preferably selected from the group consisting of colon, rectal and colorectal cancer.

It will be appreciated that treatment of ErbB related cancer according to the present invention may optionally be combined with other treatment methods known in the art (i.e., combination therapy). Thus, treatment of ErbB-related cancer may be combined with, for example, radiation therapy, antibody therapy and/or chemotherapy. Additional details on combination therapy are disclosed in U.S. Pat. No. 6,417,168 U.S. Pat. Appl. Publication Nos. 20020041865 and 20030086930, PCT Appl. No. 9960023; and in Horton Cancer Control. 2002 November-December;9(6):499-507; Denny Pharmacol Ther. 2002 February-March;93(2-3):253-61; Dieras Oncology. 2001;61 Suppl 2:43-9.; Baselga Oncology. 2001;61 Suppl 2:1421. The invention therefore additionally provides a method of treating cancer that expresses ErbB-2 comprising administering to a human a therapeutically effective amount of an a therapeutic agent as described herein and a therapeutically effective amount of a second drug selected from the group consisting of an EGFR targeted drug and a tyrosine kinase inhibitor, wherein the agent according to the present invention and the second drug are administered separately or in combination, and in either order.

The therapeutic agents of the present invention can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections. Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include pus fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Immunogenic Compositions

A therapeutic agent according to the present invention may optionally be a molecule, which promotes a specific immunogenic response against at least one of the polypeptides of the present invention in the subject. The molecule can be polypeptide variants of the present invention, a fragment derived therefrom or a nucleic acid sequence encoding thereof. Although such a molecule can be provided to the subject per se, the agent is preferably administered with an immunostimulant in an immunogenic composiiton. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes into which the compound is incorporated (see e.g., U.S. Pat. No. 4,235,877). Vaccine preparation is generally described in, for example, M. F. Powell and M. J. Newman, eds., “Vaccine Design (the subunit and adjuvant approach),” Plenum Press (NY, 1995).

Illustrative immunogenic compositions may contain DNA encoding one or more of the polypeptides as described above, such that the polypeptide is generated in situ. The DNA may be present within any of a variety of delivery systems known to those of ordinary skill in the art, including nucleic acid expression systems (see below), bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the subject (such as a suitable promoter and terminating signal). Bacterial delivery systems involve the administration of a bacterium (such as Bacillus Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et at., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

It will be appreciated that an immunogenic composition may comprise both a polynucleotide and a polypeptide component. Such immunogenic compositions may provide for an enhanced immune response.

Any of a variety of immunostimulants may be employed in the immunogenic compositions of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quit A. Cytokines, such as GM-CSF or interleukin-2, -7, or -12, may also be used as adjuvants.

The adjuvant composition may be designed to induce an immune response predominantly of the Th1 type. High levels of Th1-type cytokines (e.g., IFN-.gamma., TNF.alpha., IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of an immunogenic composition as provided herein, the subject will support an immune response that includes Th1- and Th2-type responses. The levels of these cytokines may be readily assessed using standard assays. For a review of the families of cytokines, see Mosmann and Coffinan, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720.

A delivery vehicle may be employed within the immunogenic composition of the present invention to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Dendritic cells are highly potent APCs (Banchereau and Steinman, Nature 392:245-251, 1998) and have been shown to be effective as a physiological adjuvant for eliciting prophylactic or therapeutic antitumor immunity (see Timmeman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within an immunogenic composition (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNF.alpha. to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNF.alpha., CD40 ligand, LPS, flt3 ligand and/or other compound(s) that induce differentiation, maturation and proliferation of dendritic cells.

Dendritic cells are categorized as “immature” and “mature” cells, which allows a simple way to discriminate between two well characterized phenotypes. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with at least one polynucleotide encoding a polypeptide of the present invention, such that variant II, or an immunogenic portion thereof, is expressed on the cell surface. Such transfection may take place ex vivo, and a composition comprising such transfected cells may then be used for therapeutic purposes, as described herein. Alternatively, a gene delivery vehicle that targets a dendritic or other antigen presenting cell may be administered to the subject, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with a polypeptide of the present inventio, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule) such as described above. Alternatively, a dendritic cell may be pulsed with a non-conjugated immunological partner, separately or in the presence of the polypeptide.

Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used.

Briefly, SPECT images are created by detecting high-energy photons (gamma rays) that are produced by synthetic radioactive atoms injected into the patient (for example contained in a splice variant and/or antibody according to the present invention). The most commonly used radioactive atom for SPECT is technetium-99, which has a half-life of 6 hours, which can optionally be linked to the splice variants and/or antibodies as described above. SPECT can provide both anatomical information, such as the site of metastases of tumors, as well as functional information about blood flow and cell metabolism. For example, U.S. Pat. No. 6,696,686 describes the use of SPECT for detection of breast cancer, and is hereby incorporated by reference as if fully set forth herein.

Similarly, PET involves the detection of gamma rays but with shorter-lived isotopes (less than 2 hours typically). These short-lived positron-emitting atoms are injected into the patient. When positrons travel short distances in tissues, they collide with nearby electrons, creating two gamma rays that travel in opposite directions. The pair of rays can be detected with gamma detectors surrounding the patient. These data are then processed to form a three dimensional image.

The most common positron emitter is fluorine-18, which is currently used to label deoxyglucose, a form of glucose. However, optionally it could also be used to label an antibody and/or splice variant according to the present invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### 5 Variants I and II of ErbB-2 are Ubiquitously Expressed

RNA extraction—Total RNA was extracted from cells using the Tri-Reagent (Molecular Research Center Inc., Cincinnati, Ohio).

Cell lines—The following cell lines were used: T47D (ATCC HTB-133, Manassas, Va.), BT474 (ATCC HTB-20 Manassas, Va.), MCF7 (ATCC HTB-22 Manassas, Va.), SK-BR-3 (HTB-30 Manassas, Va.), Calu-3 (ATCC HTB-55 Manassas, Va.), ES-2 (ATCC CRL-1978 Manassas, Va.), DU145 (ATCC HTB-81 Manassas, Va.), H1299 (ATCC CRL-5803 Manassas, Va.), HT29 (ATCC HTB-38 Manassas, Va.), PANC-1 (ATCC CRL-1469 Manassas, Va.), SNU-1 (ATCC CRL-5971 Manassas, Va.) and T24 (ATCC HTB-24 Manassas, Va.).

Primers RT reaction and PCR conditions—Table 3, below, lists the oligonucleotide primers which were used to detect wild-type ErbB-2, variant I, variant II and the housekeeping gene ATP synthase 6 (GenBank Accession No. AF368271).

TABLE 3

Nucleotide
coordinates

| | |
|---|---|
| Oligonucleotide sequence/ | on target |

| | | | |
|---|---|---|---|
| (SEQ ID NO:) | Orientation | Target sequence | sequence |

| | | | |
|---|---|---|---|
| 5′-gtctctgcggtggttggcattc-3′/(17) | F | Full-length ErbB-2 | 2113 |
| | | GenBank Accession | |
| | | NO. gi:10181232 | |
| 5′-gagcggccaagtccctgtgtac-3′/(18) | R | Full-length ErbB-2 | 2696 |
| | | GenBank Accession | |
| | | NO. gi:10181232 | |
| 5′-agaatggctcagtgacctgttttg-3′/(19) | F | Variant I | 1859 |
| 5′-ctcttctcagtctcccttgacttc-3′/(20) | R | Variant I | 2403 |
| 5′-atggagctggcggccttgtg-3′/(29) | F | Variant IV | 151 |
| 5′-cacagtggaagagtgggtgggaaggg-3′/(30 | R | Variant IV | 2093 |
| 5′-catttctgccggagagctttgatg-3′/(21) | F | Variant II | 1347 |
| 5′-caggtaggacccagctagagaag-3′/(22) | R | Variant II | 1919 |
| 5′-cagtgattataggctttcgctctaa-3′/(23) | F | ATP-Synthase 6 | 508 |
| 5′-cagggctattggttgaatgagta-3′(24) | R | ATP-Synthase 6 | 642 |

Reverse transcription reactions were carried out with equivalent amounts of RNA in a final volume of 25 μl. A mix containing 150 ng of random hexamer primers (Invitrogen, Carlsbad, Calif.), 1 μg of total RNA and 500 μM of each of four deoxynucleoside triphosphates were heated to 65° C. for 5 min. After adding 200 units of reverse transcriptase (Invitrogen), the reaction mixture was incubated at 42° C. for 60 min. PCR was carried out in a final volume of 25 μl containing 50 pmol of each of the oligonucleotide primers, 1 μl of RT solution and 1.25 units of Taq polymerase [Takara Ex Taq™ Hot Start Version (Takara Shuzo Co., LTD, Japan)]. Amplification was carried out by an initial denaturation step at 98° C. for 10 s followed by 35 cycles of [94° C. for 30 s, 66° C. for 30 s, and 72° C. for 0.5 min]. At the end of the PCR amplification, products were analyzed on agarose gels stained with ethidium bromide and visualized with UV light.

Results

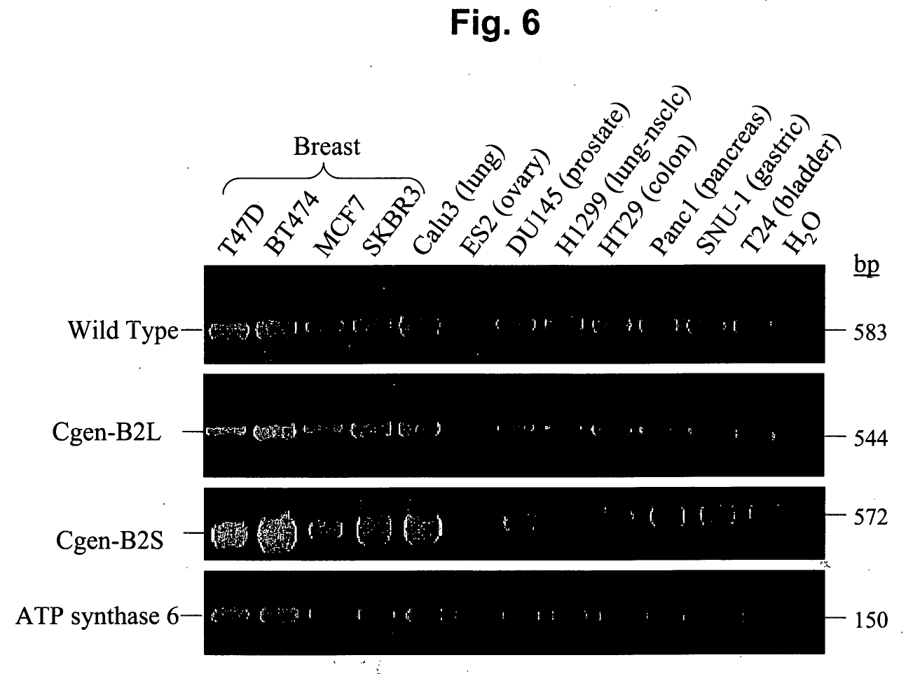

As shown in FIG. 6, amplification reactions confirmed expression of the two novel variants of ErbB-2 of the present invention, variant I (Cgen-B2S) and variant II (CgenB2S). Similarly to wild-type ErbB-2, hardly any expression of the isoforms was detected in the ovarian cell line, ES2. Interestingly, while variant 1 exhibited comparable levels of expression in all examined cell lines, the pattern of expression of the short isoform (variant II) was more heterogenic, showing almost no expression in the lung muscle cell line H1299 and almost no expression in the colon cell line HT29. Sample homogeneity was confirmed by RT-PCR of the housekeeping gene ATP SYNTHASE 6 (GenBank Accession No. AF368271).

Altogether these results validate the expression of the new ErbB-2 isoforoms of the present invention in various cell lines and tissues.

### Example 2

### Expression of ErbB-2 Transcripts in Normal and Cancerous Breast Tissues

The ErbB-2 markers of the present invention were tested with regard to their expression in cancerous and non-cancerous breast tissue samples. A description of the samples used in the panel is provided in Table 4 below. Real-time RT-PCR analysis was then performed as described below.

Materials, and Experimental Procedures

Tissue samples—Table 4, below, lists tissue samples used for real-time RT PCR analysis.

TABLE 4

| sample rename | Lot no | source | pathology | grade | age/sex | TNM | stage |
|---|---|---|---|---|---|---|---|
| 52-B-ILC G1 | A605360 | Biochain | Invasive Lobular | 1 | F/60 | | |
| | | | Carcinoma | | | | |
| 51-B-IDC G1 | A605361 | Biochain | IDC | 1 | F/79 | | |
| 6-A-IDC G1 | 7238T | ABS | IDC | 1 | F/60 | T2N0M0 | stage 2A |
| 7-A-IDC G2 | 7263T | ABS | IDC | 2 | F/43 | T1N0M0 | stage 1 |
| 12-A-IDC G2 | 1432T | ABS | IDC | 2 | F/46 | T2N0M0 | stage 2A |
| 13-A-IDC G2 | A0133T | ABS | IDC | 2 | F/63 | T2N1aMx | |
| 14-A-IDC G2 | A0135T | ABS | IDC | 2 | F/37 | T2N2Mx | |
| 15-A-IDC G2 | 7259T | ABS | IDC | 2 | F/59 | T3N1M0 | stage 3A |
| 16-A-IDC G2 | 4904020032T | ABS | IDC | 2 | NA | T3N1Mx | |
| 17-A-IDC G2 | 4904020036T | ABS | IDC | 2-3 | NA | T3N1Mx | |
| 43-B-IDC G2 | A609183 | Biochain | IDC | 2 | F/40 | | |
| 44-B-IDC G2 | A609198 | Biochain | IDC | 2 | F/77 | | |
| 45-B-IDC G2 | A609181 | Biochain | IDC | 2 | F/58 | | |
| 48-B-IDC G2 | A609222 | Biochain | IDC | 2 | F/44 | | |
| 49-B-IDC G2 | A609223 | Biochain | IDC | 2 | F/54 | | |
| 50-B-IDC G2 | A609224 | Biochain | IDC | 2 | F/69 | | |
| 53-B-IDC G2 | A605151 | Biochain | IDC | 2 | F/44 | | |
| 54-B-IDC G2 | A605353 | Biochain | IDC | 2 | F/41 | | |
| 55-B-IDC G2 | A609179 | Biochain | IDC | 2 | F/42 | | |
| 61-B-IDC G2 | A610029 | Biochain | IDC | 2 | F/46 | | |
| 62-B-IDC G2 | A609194 | Biochain | IDC | 2 | F/51 | | |
| 47-B-IDC G2 | A609221 | Biochain | IDC | 2 | | | |
| 46-B-Carci G2 | A609177 | Biochain | Carcinoma | 2 | F/48 | | |
| 26-A-IDC G3 | 7249T | ABS | IDC | 3 | F/60 | T2N0M0 | stage 2A |
| 27-A-IDC G3 | 4907020072T | ABS | IDC | 3 | NA | T2N0Mx | |
| 42-A-IDC G3 | 6005020031T | ABS | IDC | 3 | NA | T1cN0Mx | |
| 31-CG-IDC | CG-154 | Ichilov | IDC | | NA | | |
| 32-A-Muc Carci | 7116T | ABS | Mucinous carcinoma | | F/54 | T2N0M0 | stage 2A |
| 35-A-N M6 | 7238N | ABS | Normal matched to | | F/60 | | |
| | | | 6T | | | | |
| 36-A-N M7 | 7263N | ABS | Normal matched to | | F/43 | | |
| | | | 7T | | | | |
| 39-A-N M15 | 7259N | ABS | Normal matched to | | F/59 | | |
| | | | 15T | | | | |
| 40-A-N M12 | 1432N | ABS | Normal matched to | | F/46 | | |
| | | | 12T | | | | |
| 41-A-N M26 | 7249N | ABS | Normal matched to | | F/60 | | |
| | | | 26T | | | | |
| 56-B-N | A609235 | Biochain | Normal PM | | F/59 | | |
| 57-B-N | A609233 | Biochain | Normal PM | | F/34 | | |
| 58-B-N | A609232 | Biochain | Normal PM | | F/65 | | |
| 59-B-N | A607155 | Biochain | Normal PM | | F/35 | | |
| 60-B-N | A609234 | Biochain | Normal PM | | F/36 | | |
| 63-Am-N | 26486 | Ambion | Normal PS | | F/43 | | |
| 64-Am-N | 23036 | Ambion | Normal PM | | F/57 | | |
| 65-Am-N | 31410 | Ambion | Normal PM | | F/63 | | |
| 66-Am-N | 36678 | Ambion | Normal PM | | F/45 | | |
| 67-Am-N | 073P010602086A | Ambion | Normal PM | | F/64 | | |

RNA preparation —RNA was obtained from BioChain Inst. Inc. (Hayward, Calif. 94545 USA www.biochain.com), ABS (Wilmington, Del. 19801, USA, http://www.absbioreagents.com) or Ambion (Austin, Tex. 78744 USA, http://www.ambion.com). Alternatively, RNA was generated from tissue samples using TR1-Reagent (Molecular Research Center), according to Manufacturer's instructions. Tissue and RNA samples were obtained from patients, from postmortem or from post breast reduction surgery. Total RNA samples were treated with DNaseI (Ambion) and purified using RNeasy columns (Qiagen).

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8).

Real-Time RT-PCR analysis—cDNA (5 μl), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. The relative quantity was calculated using the equation Q=efficiency\hat{ }-Ct. The efficiency of the PCR reaction was calculated from a standard curve, created by using serial dilutions of reverse transcription (RT) reactions prepared from RNA purified from 5 cell lines (HCT116, H1299, DU145, MCF7, ES-2). To minimize inherent differences in the RT reaction, the resulting relative quantities were normalized to the geometric mean of the relative quantities of several housekeeping (HSKP) genes.

Results

Expression of ErbB-2 transcripts was measured by real time PCR. ErbB-2 Wild Type (WT) was detectable by an oligonucleotide probe (SEQ ID NO:47). ErbB-2-long variant (for example as depicted in SEQ ID NO: 1, 13 or the unique sequence SEQ ID NO:7) was detectable by SEQ ID NO:50, and ErbB-2-short (for example as depicted in SEQ ID NO: 3, 15, and the unique sequence as depicted in SEQ ID NO: 8) was detectable by SEQ ID NO:53. In parallel the expression of four housekeeping genes—PBGD (GenBank Accession No. BC019323; amplicon—SEQ ID NO: 38), HPRTI (GenBank Accession No. NM_{—}000194; amplicon—SEQ ID NO: 35), G6PD (GenBank Accession No. NM_{—}000402; amplicon—SEQ ID NO: 44) and SDHA (GenBank Accession No. NM_{—}004168; amplicon—SEQ ID NO: 41), was measured similarly. For each RT sample, the expression of SEQ ID NOs: 47, 50 and 53 was normalized to the geometric mean of the quantities of the housekeeping genes. The normalized quantity of each RT sample was then divided by the median of the quantities of the normal post-mortem (PM) and post breast reduction surgery (PS) samples (Sample Nos. 56-60, 63-67, Table 4, above) determined separately for each SEQ ID, to obtain a value of fold up-regulation for each transcript in each sample relative to median of the normal samples.

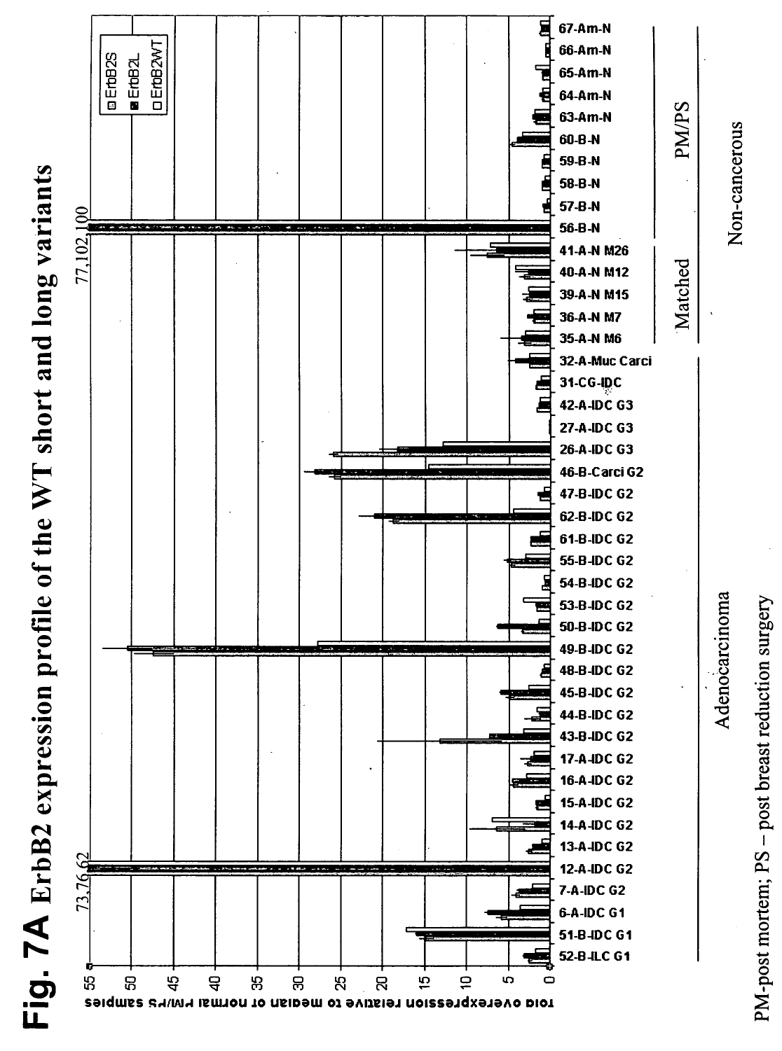

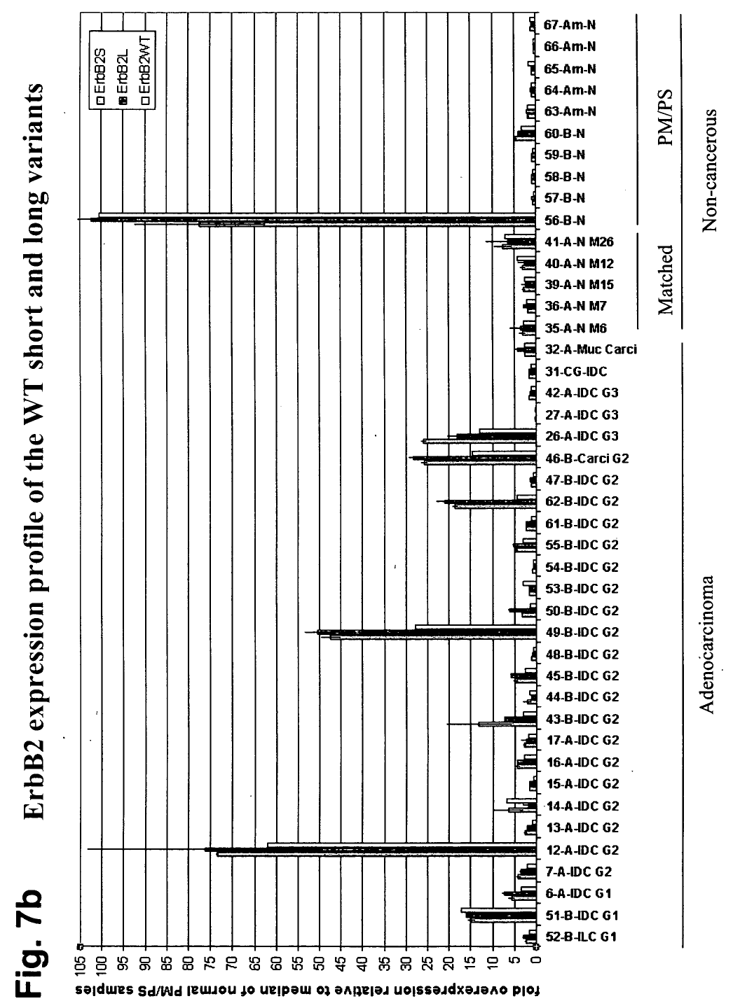

FIGS. 7a-b are histograms showing over expression of the above-indicated ErbB-2 transcripts (B2S and B2L) in cancerous breast samples relative to normal breast samples. The histogram is shown twice, with a small scale (FIG. 7a) and a large scale (FIG. 7b) for the sake of clarity only. The error bars indicate the minimum and maximum values of the different measurements in the two experiments.

As is evident from FIGS. 7a-b, the expression of ErbB-2 B2S and B2L variants detectable by the oligonucleotides of SEQ ID NOs: 50 and 53 in cancer samples was significantly higher than in the non-cancerous samples (Sample Nos. 35,36,39-41,56-60, 63-67, Table 4). Notably, for the ErbB-2 B2L and B2S variants, an over-expression of at least 5 fold was found in 12 out of 28 adenocarcinoma samples, while for the WT, an over-expression of at least 5 fold was found only in 6 out of 28 adenocarcinoma samples. One of the normal samples (Sample No. 56, Table 4, above) showed high over-expression of all checked ErbB-2 transcripts. Since over-expression of ErbB-2 WT was observed in this sample, and also over-expression of hTERT which is known to be expressed only in cancerous cells (data not shown), it is thought that this sample is either contaminated with cancerous cells, or alternatively may have been marked by mistake as a normal sample.

The B2S and B2L variants were over expressed at least 5 fold also in 1 of the 5 matched normal samples. Since matched samples are histologically non-cancerous tissue that surrounds the tumor, such samples could have been contaminated with cancer or pre-cancer cells.

### Example 3

### Production of Polyclonal Antibodies Specific to ErbB-2 Variants I (B2L; SEQ ID NO: 1, 2) and II (B2S; SEQ ID NO: 3, 4).

Materials and Experimental Procedures

Animals—Two rabbits were injected to prepare antibodies for Erb2-1 (B2L; rabbit numbers 1563 and 1564). Two rabbits were injected to prepare antibodies for Erb2-2 (B2S; rabbit numbers 1565 and 1566). All animal care, handling and injections were performed by Sigma (Israel).

Peptide synthesis—The peptides which were used for rabbit immunization were as follows: CPSLPHWMLGGHCCREGTP (SEQ ID NO: 54), a sequence taken from the unique tail of the ErbB-2 variant I (B2L; SEQ ID NO: 2) splice variant, and CQHTAVPRGPWQQRSWT (SEQ ID NO: 55), a sequence taken from the unique tail of the ErbB-2 variant II (B2S; SEQ ID NO: 4) splice variant. Peptides were synthesized by Sigma Chemicals (Israel).

KLH conjugation—This process was performed by Sigma (Israel).

Immunization—Rabbits were immunized with two peptides, both of which were KLH conjugated and then purified.

Antibody purification—Anti-B2L and anti-B2S antibodies were purified from rabbit serum by ammonium sulfate precipitation. Briefly, a saturated solution of ammonium sulfate was prepared by adding 380 gr to 500 ml water and boiling the solution. The serum was thawed and centrifuged at 10 000 rpm, 4° C. for 5 min. 1 volume (vol) PBS was added to each vol serum, and stirred at 4° C. 1 volume of saturated ammonium sulfate was then added while stirring for at least 2 h hour on ice. The solution was centrifuged 15 min at 10000 rpm at 4° C. to precipitate immunoglobulins (e.g., IgG). The pellet was resuspended in 5 ml PBS and dialyzed overnight at 4° C. against PBS+0.05% azide. The precipitated serum was filtered with a 0.45 μm filter (Millipore, USA).

Affinity purification was then performed with the peptide against which the respective antibodies were raised as described above, SEQ ID NOs: 54 or 55 for the variants B2L or B2S, respectively, in an immunoaffinity column, linked to sulfolink beads (Pierce # 20401). The column was prepared according to manufacturer's instructions. The serum to be purified was mixed with sulfolink beads and incubated under gentle shaking (1 h at R.T. and 2 h at 40° C.), after which the beads were packed into a column.

The column was washed with TRIS 100 mM, followed by a second wash with binding buffer containing 0.5M NaCl. The IgG fraction was eluted by applying elution buffer including 0.1M Glycine pH3 (fraction size: 0.5 ml), followed by phosphate buffer 100 mM pH 11 to elute another fraction of IgG. In order to neutralize acidic or basic pH, 1/10 volume TRIS 1M pH=8 was added to collecting tubes prior to adding elution buffer to the column. The antibodies were dialyzed overnight against a buffer of PBS and 0.025% azide, and then frozen for storage.

Western Blot analysis—see Example 5 below.

Results

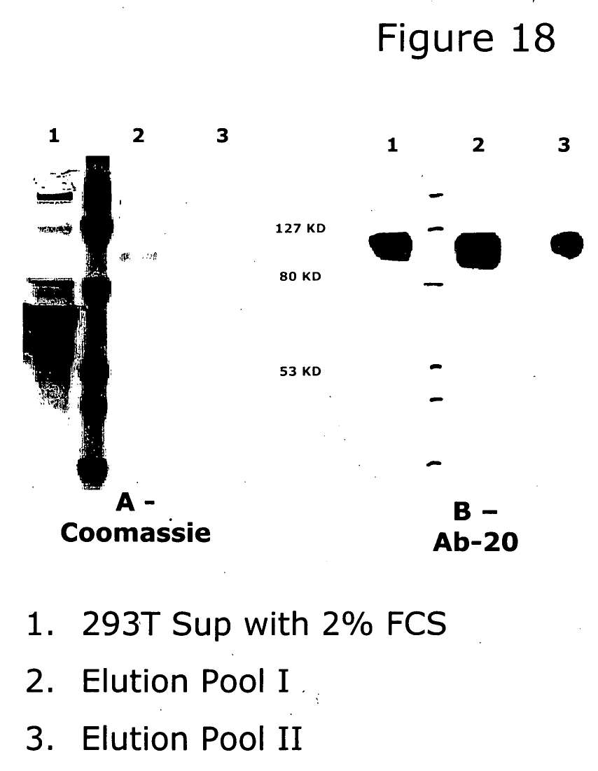

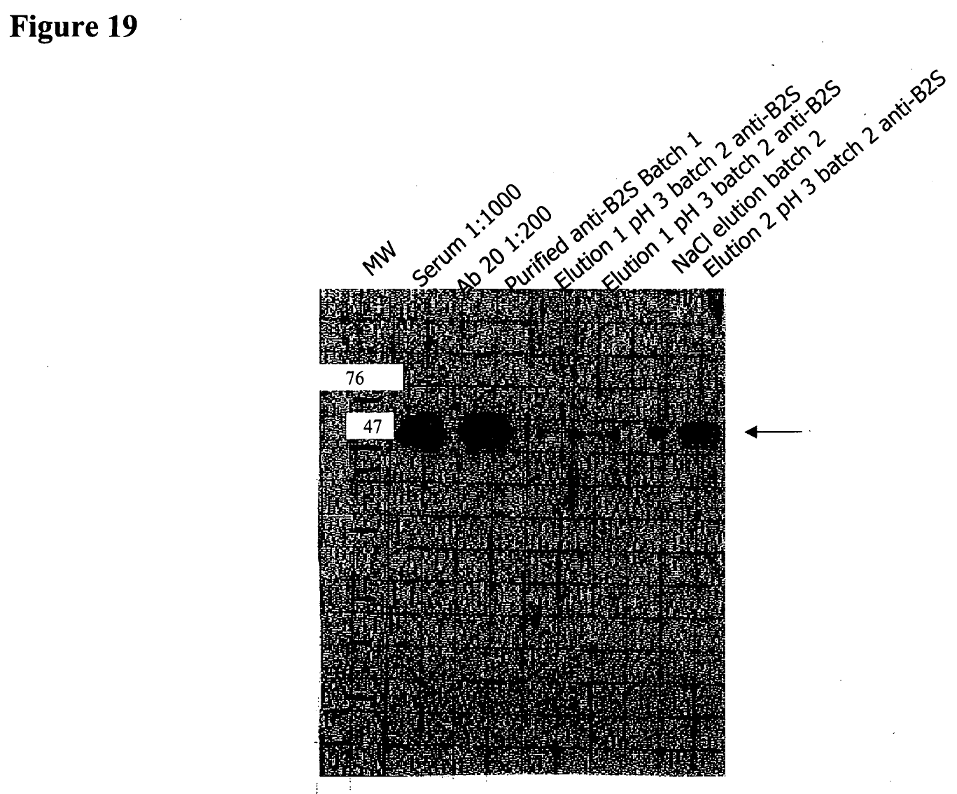

Antibodies which are capable of specifically binding variants I and II of ErbB2 generated as described above, were qualified by Western Blot analysis, results of which are presented in FIG. 18 (for B2S, variant II) and 19 (for B2L, variant I). As shown, antibodies generated according to the present invention specifically recognized the novel ErbB-2 variants of the present invention as is evident by the detection of a single band at the apparent molecular weight of about 67 kDa (FIGS. 18a-b) and about 100 kDa (FIG. 19) for variants II and I respectively.

### Example 4

### Cloning and Purification of the ErbB-2 Variants I (B2L; SEQ ID NO: 1, 2) and II (B2S; SEQ ID NO: 3, 4)

This Example describes cloning of splice variants in bacteria (B2S) or in mammalian expression systems. Different such expression systems were used to check expression efficiency, amount of expressed proteins produced and also to characterize the expressed proteins. Expressed proteins were also purified.

### Example 4a

Cloning of B2S and B2L Variants

The B2L and B2S splice variants cloning fragments were prepared by PCR amplification using TaKaRa Hot-Start Ex-Taq™ (Chemicon Int. CA. USA) under the following conditions: 2.5 μl—Ex-Taq X10 buffer; 5 μl—cDNA; 2 μl—dNTPs (2.5 mM each); 0.5 μl—Ex-Taq enzyme; 14 μl—H₂O; and 0.5 μl—of each primer (25 μM) in a total reaction volume of 25 μl; with a reaction program of 5 minutes in 95° C.; 40 cycles of: 30 seconds at 94° C., 45 seconds at 68° C., 60 seconds at 72° C. and 10 minutes at 72° C. Primers which include specific sequences of the nucleotide sequence corresponding to the splice variants and Gateway™ BP recombination tails were used and are listed in Table 5, below.

TABLE 5

SEQ
ID

| | | |
|---|---|---|
| Primer ID: | Sequence | NO: |

| | | |
|---|---|---|
| I. Gateway forward | 5′-GGGG ACA AGT TTG TAC AAA | 54 |
| attB1 primer | AAA GCA GGC TNN | |
| 2a. B2S/B2L forward | 5′-GGGG ACA AGT TTG TAC AAA | 55 |
| primer | AAA GCA GGC T cc atg gag | |
| | ctg gcg gcc ttg tg-3′ | |
| 3. Gateway reverse | 5′-GGGG AC CAC TTT GTA CAA | 56 |
| attB2 primer | GAA AGC TGG GTN | |
| 5. B2S reverse | 5′-GGGG AC CAC TTT GTA CAA | 57 |
| primer | GAA AGC TGG Gta ggc atg gct | |
| | gtg ggc tgg ag-3′ | |
| 5a. B2L reverse | 5′-GGGG AC CAC TTT GTA CAA | 58 |
| primer | GAA AGC TGG Gta tgg gtt tcc | |
| | tag cca tct ttc ctt gc-3′ | |

Capital letters are the sequence from the Gateway primer, while the small letters (apart from underlined nucleotides) are from the ErbB-2 variant sequences; underlined nucleotides are bridging nucleotides.

PCR products were run in a 2% agarose gel, TBEX1 solution at 150 V, and extracted from gel using QiaQuick™ gel extraction kit (Qiagen™). The extracted DNA products were sequenced by direct sequencing using Gateway primers (Forward=primer number 1 above; and Reverse=primer number 3 above). Error-free inserts were introduced into Gateway™ entry clone (Invitrogen™) by a BP clonase reaction (according to the manufacturer's protocol). BL21star competent bacteria (Invitrogen Corp.) were transfected with the resulted clones using the following protocol:

5 μl of each BP reaction product were mixed with freshly thawed 50 μl of competent BL21star cells. The mix was incubated on ice for 30 minutes and then exposed to Heat-Shock at 42° C. for 30 seconds. 450 μl of LB was added to each tube, and the tubes were incubated at 37° C. in a shaker for 1 hour.

From each transfection solution, both 50 μl and 150 μl were plated on selective LB plates containing 50 μg/ml Kanamycin. The plates were incubated at 37° C. overnight.

10 Colonies from each transcript clone that grew on the selective plates were taken for further analysis by re-plating on a selective plate and by PCR. PCR was performed using the primers which are listed in Table 6, below and are specific to the vector (pDONR), located upstream and downstream to the insert.

TABLE 6

SEQ ID

| | | |
|---|---|---|
| Primer ID: | Sequence | NO: |

| | | |
|---|---|---|
| pDONR-Forward | 5′-CGCGTTAACGCTAGCATGGAT-3′ | 32 |
| pDONR-Reverse | 5′-CACAGAGTTTTAGAGACTACAAT-3′ | 33 |

PCR products were extracted and sequenced as above, using the Gateway primers described above.

Colonies containing an error free insert (no mutations within the ORF) were grown in 2 ml LB including 50 ng Kanamycin for overnight at 37° C. Plasmids were obtained from bacterial colonies using Qiaprep™ spin miniprep kit (Qiagen). Plasmid inserts were transferred into pDEST destination vectors (Gateway™-Invitrogen) according to the manufacturer protocol. Both constructs were transferred into pDEST17™. Accurate cloning was verified by sequencing the clones' inserts.

### Example 4B

Expression of the B2S Variant in Bacteria

The coding sequence of B2S as described above (Example 4a) was inserted into the pDEST17 vector, which confers ampicillin resistance. The coding sequence coded for a protein having the 6His tag at the end (6 His residues in a row at one end of the protein), for a total of 21 additional amino acids including the amino acids encoded by the Gateway primer. The cells were competent BL21star cells. A solution of calcium chloride was used to permit the vector to enter, at 100 mM concentration. Following insertion, the sequence was verified, to be certain that cloning of the correct sequence has occurred and in the correct orientation (verification was performed by sequencing).

Bacterial cell growth and induction of protein expression—Cells containing the vector were then grown in LB medium, in ampicillin (50 μg/ml) and kanamycin (30 μg/ml; resistance to the latter is inherent to BL21star cells). Cells were grown until a value of about 0.7-1 (OD at 600 mm) was reached. This value was reached in about 4 hours for the starter, which was then diluted 100 times and grown for about 3 hours induction (B2S).

Protein extraction and purification—The His-tagged protein was purified by using nickel beads with urea buffer as follows. The cells were suspended in 1/10 volume of a denaturing lysis buffer consisting of 8 M urea, 50 Mm NaH₂PO₄, 300 mM NaCl and 10 mM imidazole. Following extensive agitation for 30 min at room temperature, the solubilized material was centrifuged for 30 min at 10,000 g at room temperature and the clarified supernatant taken for IMAC using Nickel agarose beads (Qiagen).

Purification of extracts by IMAC was effected as follows. Appropriate volumes of Nickel agarose were centrifuged to remove ethanol; the resin was then resuspended gently with water and centrifuged (two times). After discarding the water, the resin was gently resuspended with clarified extracts and binding was allowed to proceed at room temperature for at least 60 min. Following binding, the resin was washed with increasing concentrations of imidazole (20-500 mM) in respective lysis buffer to elute purified protein. Washing/elution was performed by either repeated rounds of centrifugation or after loading on disposable 5 ml polypropylene columns. Following the final elution with imidazole, resin was treated with 0.1 M EDTA to strip Nickel from the resin to assay for very strongly bound material.

Coomassie staining—See Example 5 below.

Western blotting—See Example 5 below.

Results

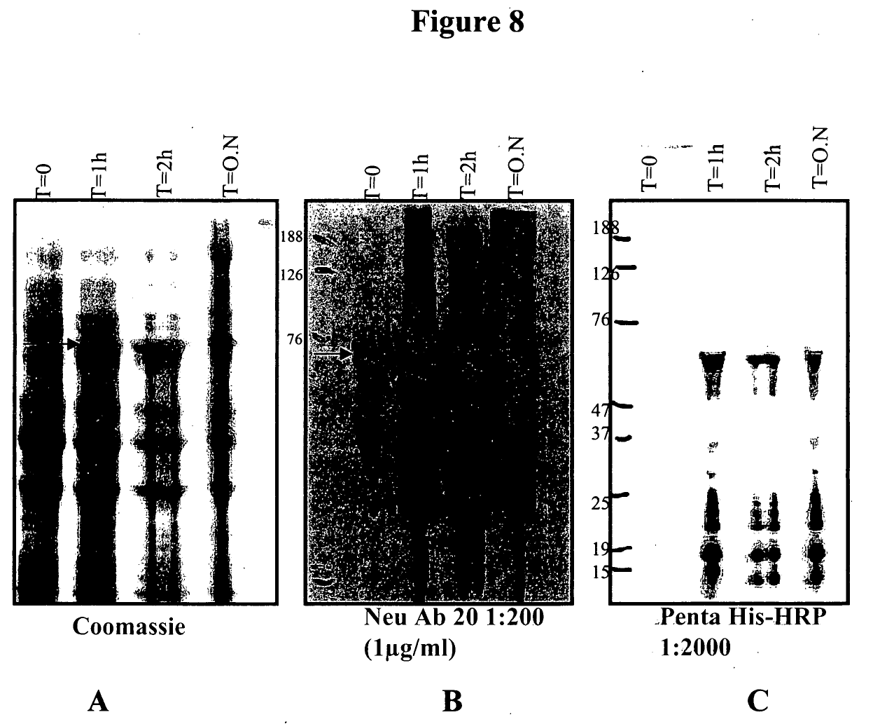

The time course of small-scale expression of B2S in BL2 I star is demonstrated in FIGS. 8a-c. The expression of the recombinant protein was detected both by Commassie staining (FIG. 8a) and by Western blot using Ab-20 ErbB-2 specific antibody (FIG. 8b) and penta His-HRP (QIAGEN) antibodies (FIG. 8c); protocols are described below with regard to Example 5 The recombinant B2S protein was highly expressed in BL21star, with expression easily detectable 1 hour, 2 hours and overnight following IPTG induction (T=1 h, T=2 h and T=O.N., respectively, in FIGS. 8a-c). The B2S protein was found to be possibly degradable in this system. A solubility test of bacterially expressed B2S, with both a Western blot, using ErbB-2 specific antibody Ab-20, and a Coomassie stained gel, of soluble versus unsoluble fractions 3 hours after the IPTG (isopropyl-β-D-thiogalactoside; Roche Diagnostics GmbH, Germany) induction and at T=0, showed that the protein mainly appeared in the insoluble fraction (data not shown). As noted before, P185 refers to the extracellular portion of ErbB-2 gene product, sp185-Her2 from Bender MedSystems GmbH (Austria).

### Example 4C

### Cloning of the ErbB-2 Variants I (B2L; SEQ ID NO: 1, 2) and II (B2S; SEQ ID NO: 3, 4) in pcDNA3 and in pIRESpuro3 Mammalian Expression Vectors

The two ErbB-2 variants (i.e., B2L and B2S) were cloned in mammalian expression vectors pcDNA3 and pIRESpuro. In both cases, no tags were added to the coding sequence, as the tag was added afterwards (see below). Cloning was initially performed in the pcDNA3 vector as follows.

mRNA from the SKBR3 cell line was isolated and treated with DNAse I, followed by reverse transcription using random hexamer primer mix and Superscript™.

Cloning into pcDNA3

The B2L and B2S splice variant cloning fragments were prepared by RT-PCR amplification using Stratagene Native Pfu DNA polymerase (Stratagene Corp. Ca. USA) under the following conditions: 2.5 μl—X10 buffer; 5 μl —cDNA; 2 μ—dNTPs (2.5 mM each); 0.5 μl—Pfu DNA polymerase; 14 μl—H₂O; and 0.5 μl—of each primer (25 μM) in a total reaction volume of 25 μl; with a two-step reaction program of 4 cycles of: 30 seconds at 94° C., 30 seconds at 60° C., 2.5 minutes at 72° C. and 20 cycles of: 30 seconds at 94° C. and 3 minutes at 72° C. followed by 10 minutes at 72° C.

Primers including splice variant specific sequences and sequences corresponding to the pcDNA3 vector (Invitrogen) are listed in Table 7, below.

TABLE 7

| | | |
|---|---|---|
| Primer ID: | Sequence | SEQ ID NO: |

| | | |
|---|---|---|
| B2L/B2S forward for | 5′-GGGGTACCACCATGGAGCTGGCGGCCTTG-3′ | 59 |
| pcDNA3 | | |
| B2L reverse primer | 5′-GACTCTCGAGTTATCAGGGGGTCCCTCCCTAC-3′ | 60 |
| for pcDNA3 | | |
| B2S reverse primer | 5′-GACTCTCGAGTTATCAGAAACAGAGGGGGAGGG-3′ | 61 |
| for pcDNA3 | | |

PCR products were run in a 1% agarose gel, TBEX1 solution at 150 V, and extracted from gel using QiaQuick™ gel extraction kit (Qiagen™). The extracted DNA products were sequenced by direct sequencing using pcDNA3 specific primers (Hy-Labs, Israel). Error-free inserts cut with the restriction enzymes KpN I and Xho I (New England Biolabs, USA). were introduced into a pcDNA3 vector cut with the same enzymes, using the LigaFast™ Rapid DNA Ligation System (Promega).

Subcloning into pIRESpuro3

The cloning procedure for pIRESpuro3 vector (Clontech) was as follows SnaBI-XbaI restriction enzymes were used to take out the B2L coding insert from pcDNA3. The 2366 bp fragment was cloned into pIREs puro3, which was previously digested with SnaBI-NheI (excising the 337 bp fragment). SnaBI cuts inside the CMVPr in both plasmids, and the sequence matches.

B2LHis Cloning (Addition of a C′ His Tag)

PCR was performed in a final volume of 50 ll containing 50 pmol of each of the oligonucleotide primers (listed below), 2 ll of DNA template (B2L pcDNA3 70 ng\ul) and 1.25 units of Taq polymerase [Platinum Pfx DNA polymerase (Invitrogen)]. Amplification was performed with an initial denaturation step at 94° C. for 3 minutes followed by 29 cycles of [94° C. for 30 s, 55° C. for 30 s, and 68° C. for 1 min]. At the end of the PCR amplification, products were analyzed on agarose gels stained with ethidium bromide and visualized with UV light.

The oligonucleotide sequences were as follows: (1) ATCCGGGGACGAATTCTGC (SEQ ID NO: 62); (2) TGCTCTAGATTATCAGTGGTGATGATGATGGTGAGGGGTCCCCTCCCTA CAG (SEQ ID NO: 63). Sequence (1) relates to B2L or B2S, while sequence (2) includes (complementary and reverses the following sites (in this order): XbaI site, 6-His tag sequence (shown with underlining) and the end of the B2L coding region (the latter was adjusted with a silent mutation to change CCC to CCT in the complementary, non-reversed sequence, both of which code for Pro, to avoid potential problems with PCR; the sequence is shown in bold face type).

The PCR product was then double digested with EcoRI and XbaI (New England Biolabs (UK) LTD), and inserted into B2S pcDNA3 previously digested with the same enzymes to give B2LHis pcDNA3.

Subcloning of B2LHis into pIRESpuro3 was performed as follows. B2L-His pcDNA3 was double digested with SnaBI and XbaI and the 2372 bp fragment was ligated into pIRESpuro3 previously digested with SnaBI and NheI. B2S-His was constructed the same way except for the following variations: DNA template (B2SpcDNA3) concentration was 40 ng/μl. The oligonucleotide sequences were were as set forth in SEQ ID NOs: 62 and 63. The inserts were fully sequenced to exclude mutations due to the PCR.

### Example 4D

Transient and/or Stable Expression of ErbB-2 B2L and B2S Variants

Transient expression experiments were done in 293T and COS7, showing a superior expression level in 293T cells. B2L was superior to B2S in expression levels, estimated as 2.5 mg B2L for each liter serum-free culture. The cell lines used were as follows: COS7 (ATCC CRL-1651); 293T (ATCC CRL-11268).

Stable cell populations were generated in CHO DUKX B11 and 293T cells by transfection with pcDNA3-B2L (Invitrogen) or pIRESpuro-B2L (Clontech), respectively. Productivity of the 293T cells was higher than CHO, similarly to the transient cultures, reaching about 1.5 mg/L/day). Scale up of cultures was done with multi-tray stacks (MT10).

Transient transfection to 293T cells and COS7 cells—For small scale transient transfection of 293T cells with pIRESpuro-B2L vector or pIRESpuromock, the FuGENE 6 transfection reagent was used (Roche, Switzerland). 2 μg of DNA were used per well in a 6 well plate (2 wells). Culture media for the different steps of transfection is described in Table 8, below.

TABLE 8

| Medium | | |
|---|---|---|
| No. | Content | Vendor |
| 1 | 10% FCS + P/S/A + Glutamine in | FCS - #04001-A Biological |
| | DMEM (Dulbecco's modified | Industries, Israel |
| | Eagle's medium) | P/S/A (PENICILLIN- |
| | | STREPTOMYCIN- |
| | | AMPHOTERICIN B(2) |
| | | solution) - #03-033-1 |
| | | Biological Industries, Israel |
| | | Glutamine - #030201 |
| | | Biological Industries, Israel |
| 2 | DMEM + Glutamine | DMEM - #01-055-1A |
| | | Biological Industries, Israel |
| 3 | DMEM | |

It should be noted that 6 μl of FUGENE was used for every 2 μg of DNA (per 6 well plate).

One day prior to transfecting, two 6 well plates were plated with 500,000 cells per well (in 2 ml medium #1).

To start the transfection process, the FuGENE 6 Transfection Reagent was warmed to ambient temperature and mixed prior to use.

6 μl of FuGENE were diluted into 100 μl DMEM (volume per each well). Next, 2 μg of DNA were added to each tube. The contents were gently mixed and incubated at room temperature (RT) for 15 minutes. 100 μl of the complex mixture was added dropwise to the cells and swirled. The cells were incubated overnight in the incubator.

Following about 24 h, transfected cells were washed with DMEM and incubated with 2 ml fresh DMEM (+glutamine, medium #2). Following 48 more hours, the supernatant was collected.

It should be noted that a similar protocol was used for transient transfection of COS7 cells with the B2L construct, including using the same construct and conditions.

For large scale transient transfection of COS7 cells with pcDNA3-B2S (DNA coding for B2S variant in the pcDNA3 vector), the FuGENE 6 transfection reagent was used (Roche, Switzerland). Cells were transfected with pcDNA3B2S in the presence or absence of pAdVAntage (Promega); the latter construct in some cases may increase the amount of protein obtained.

Cell culture media for the different steps of transfection is listed in Table 9 below.

TABLE 9

| Medium | | |
|---|---|---|
| No. | Content | Vendor |
| 1 | 10% FBS + P/S/A + Glutamine | FBS - SH30071.03 HyClone, |
| | in DMEM | USA |
| | | P/S/A - #03-033-1 Biological |
| | | Industries, Israel |
| | | DMEM - #01-055-1A |
| | | Biological Industries, Israel |
| | | Glutamine - #030201 |
| | | Biological Industries, Israel |
| 2 | P/S/A + Glutamine in DMEM | |
| 3 | DMEM | |

It should be noted that 18 μl of FUGENE was used for every 6 μg of DNA (per T175 flask, having a surface area of 175 cm²).

About 1.5×10⁶ cells were plated one day prior to transfection in about 20 ml medium (DMEM+10% FBS+P/S antibiotics) per T175 flask (without pAdvantage; with pAdvantage about 2×10e6 cells were plated). FuGENE 6 Transfection Reagent was warmed to ambient temperature and mixed prior to use. 18 μl of FuGENE was diluted into medium DMEM to a final volume of 250 μl, after which 4 μg pcDNA3-B2S and (when present) 3 μg vector from pAdVAntage were added. The contents were gently mixed and incubated at room temperature (RT) for 15 minutes. 250 μl of the complex mixture was added dropwise to the cells and swirled. The cells were incubated overnight in the incubator. After about 24 h, transfected cells were washed with DMEM and incubated with 25 ml fresh DMEM (+glutamine, without serum). After 48 more hours, the supernatant was collected.

Small scale transient transfection of 293T cells with pcDNA3-B2S+pAdvantage in the presence of the FuGENE 6 transfection reagent (Roche, Switzerland) was effected using the media listed in Table 10, below.

TABLE 10

| Medium | | |
|---|---|---|
| No. | Content | Vendor |
| 1 | 10% FCS + P/S/A + Glutamine | FCS - #04001-A Biological |
| | in DMEM | Industries, Israel |
| | | P/S/A - #03-033-1 Biological |
| | | Industries, Israel |
| | | DMEM - #01-055-1A |
| | | Biological Industries, Israel |
| | | Glutamine - #030201 |
| | | Biological Industries, Israel |
| 2 | DMEM + Glutamine | |
| 3 | DMEM | |

It should be noted that 3 μl of FUGENE was used for every 1.5 μg of pcDNA3 B2S DNA (per 6 well plate). One day before transfection, two 6 well plates were plated with 200,000 cells per well (in 2 ml medium). FuGENE 6 Transfection Reagent was warmed to ambient temperature and mixed prior to use. 3 μl of FuGENE was diluted into medium DMEM 100 μl, after which 1.5 μg pcDNA3-B2S and 1.5 μg pAdVAntage vector were added. The contents were gently mixed and incubated at room temperature (RT) for 15 minutes. 100 μl of the complex mixture was added dropwise to the cells and swirled. The cells were incubated overnight in the incubator. After about 24 h, transfected cells were washed with DMEM and incubated with 2 ml fresh DMEM (+glutamine). After 48 more hours, the supernatant was collected.

Stable transfection of 293T cells and CHO cells—For stable transfection of 293T cells with pIRESpuro-B2L vector, the FuGENE 6 transfection reagent was used (Roche, Switzerland). The stable transfection was similar to the transient transfection described above, only 5 μg/ml puromycin were used for the selection process. Culture media for the different steps of transfection is described in Table 11, below.

TABLE 11

| Medium | | |
|---|---|---|
| No. | Content | Vendor |
| 1 | 10% FCS + P/S/A + Glutamine | FBS - SH30071.03 HyClone, |
| | in DMEM | USA |
| | | P/S/A - #03-033-1 Biological |
| | | Industries, Israel |
| | | DMEM - #01-055-1A |
| | | Biological Industries, Israel |
| | | Glutamine - #030201 |
| | | Biological Industries, Israel |
| 2 | 10% FCS + P/S/A + Glutamine | |
| | in DMEM + 5 μg/ml Puromycin | |
| 3 | DMEM + Glutamine | |
| 4 | DMEM | |

It should be noted that 6 μl of FUGENE was used for every 2 μg of DNA (per 6 well plate).

One day prior to transfection, two 6 well plates were plated with 500,000 cells per well (in 2 ml medium #1). To start the process, the FuGENE 6 Transfection Reagent was warmed to ambient temperature and mixed prior to use. 6 μl of FuGENE were diluted into 100 μl DMEM (volume per each well). Next, 2 micrograms of DNA were added to each tube. The contents were gently mixed and incubated at room temperature (RT) for 15 minutes. 100 μl of the complex mixture was added dropwise to the cells and swirled. The cells were incubated overnight in the incubator. Following about 24 h, transfected cells were split and transfered into selection medium.

The following protocol was used for the stable transfection of CHO-dhfr(−) B11 cells (kind gift of Prof. Chasin, The Hebrew University, Jerusalem, Israel) with B2L splice variant, by using LipoFectamine as the transfection reagent (Invitrogen Cat: 50470 Lot: 1167874). The cells were grown in IMDM+HT+10% FBS (HT-Hypoxantine Thymidine #030851B Biological Industries, Israel. IMDM-Iscove's Modified Dulbecco's Medium #010581A, Biological Industries, Israel).

The process began as follows. The DNA was filtered for sterilization and OD was determined. Transfections included the following cells and vectors: pcDNA3-B2L+pSVE2-DHFR into CHO-dhfr(−) B11 cells; and pcDNA3+pSVE2-DHFR into CHO-dhfr(−)B11 cells (mock).

The transfections were performed with a molar ratio of 10:1 between expression and selection vectors; for each transfection in a 6 well plate, a total amount of 2 μg DNA was used.

The procedure was performed as follows. The day prior to transfection, 300,000 cells were seeded per well in 6-well plates with 2 ml medium (see above). The cells were incubated at 37° C. in a CO₂ incubator for 18-24 hours. For each well in a transfection, 2 μg of DNA were diluted into 100 μl of medium without serum. For each well in a transfection, 10 μl of Lipofectamine reagent (GIBCO-BRL) were diluted into 100 μl of medium without serum (90 μl medium).

The DNA and the lipofectamine were mixed by pipetting up and down, followed by 15 min incubation at room temperature. The cells were washed with medium without serum, to remove serum, followed by adding 0.8 ml medium without serum to each well, drop wise, 200 μl per well, followed by swirling to mix and incubating for 4 hours at 37° C.

The lipofectamine mix was washed from the cells, followed by adding medium with serum and incubating for 48-72 hours. Following 72 h, the medium was changed into medium with dialyzed FBS (without HT), which is the first selection condition.

After selection of the resistant cells, the medium was changed to IMDM supplemented with dialyzed FBS and 1 mg/ml G418, which is the second selection condition.

Next, MTX (Methotrexate #M8407, SIGMA, Israel) amplification of B11-/B2L was performed, using the cells from the above procedure. Amplification of cells from the above transfection (selected in dFBS+1 mg/ml G418 as the second selection condition) was performed in MTX (100 nM-1000 nM) as follows.

The following media were used: growth medium including IMDM+10% dFBS+1 mg/ml G418; and freezing Medium including fresh medium (IMDM) with 20% serum and 10% DMSO.

The amplification procedure was performed as follows. Amplification started at 100 nM MTX, continued to 250, 500 and finished at 1000 nM MTX. 500,000 cells were transferred at each amplification stage. Cells were grown at least one week in each amplification stage. First, a cell culture was grown to about 90% confluence. Culture medium was removed, followed by rinsing twice the cell layer briefly with Trypsin-EDTA solution (Biological Ind 03-052-1), leaving about 1 ml in the flask. Next, cells were observed under a microscope until cell layer is dispersed (about 5-15 min). 10 ml fresh medium was added to the flask and cells were aspirated by gently pipetting. A cell count was made from an aliquot, followed by transferring 500,000 cells to medium containing 100 nM MTX. Flasks that became confluent in less than 7 days were reseeded at the same concentration. If ells created clones, they were reseeded again at the same concentration to disperse the cells.

When a flask was about 70%-80% confluent, the medium was replaced with fresh medium. When a flask was confluent (and at least 7 days at the same MTX concentration), aliquots were removed from the sup for testing, and 500,000 cells were seeded at the next concentration. The remaining cells were frozen. This process was repeated with first 500 nm and then in 1000 nM MTX.

To ensure that true clones were obtained, the cells were cloned by limiting dilutions. The process was as follows.

Cells were grown in a TC flask in the presence of medium containing IMDM+10% dFBS+1 mg/mlG418+100 nm MTX to about 70% confluence, trypsinized and resuspended in growth medium. The cell suspension was mixed and cell density was counted, preferably in the range of 0.2-1.2×10⁶ cells/ml.

For an initial limiting dilution, cells were diluted with growth medium (10 ml medium IMDM+10% dFBS+1 mg/mlG418+100 nm MTX) to obtain cell suspensions of 5 cell/well, and were then seeded in 96-wells plates, 150 μl per well.

Confirmation of clonality was then performed as follows. After 12 weeks, cells were examined with a microscope for wells containing cells (protocloning). After the colonies became visible (in about 4 weeks), the number of colonies per plate was counted to statistically calculate the probability of clonality.

Clones were screened after the cells in the wells were grown, in order to detect the presence of the recombinant product by ELISA (or equivalent). Positive clones were transferred into a well of a 24-well plate containing 1 ml growth media per well.

The limiting dilution process was performed again as follows, to obtain a clear limiting dilution. Cells were seeded at concentration of 0.3 cell/well and were grown in the presence of medium containing 10% dFBS+1 mg/mlG418+100 nM MTX. The number of wells having cells were then counted to determine whether the limiting dilution had in fact occurred at the desired concentration, according to a statistical estimation.

### Example 4E

Expression Analysis of Transiently and Stably Transfected ErbB-2 Variants

Expression of ErbB-2 variants in the various mammalian cell systems described hereinabove was tested by ELISA and Western Blotting.

Materials and Experimental Procedures

ELISA—A commercial ELISA for ErbB-2 was used to quantify B2L levels during expression and in purification fractions. Extracellular portion of ErbB-2 (p-185 standard, previously described) was used as standard. ELISA kit was obtained from Bender MedSystems GmbH (Austria) and prepared according to manufacturer instructions.

Western Blotting—see Example 5 below.

Results

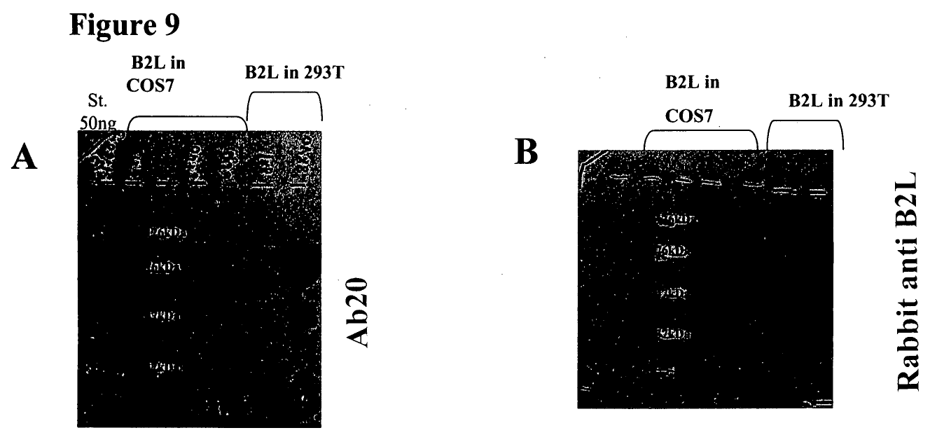

FIGS. 9a-b demonstrate Western blot analysis of transiently expressed B2L variant in COS7 and in 293T cell lines. FIG. 9a shows the expression of B2L using the a general anti p185 antibody (Ab-20), while FIG. 9b shows the specificity of the production of the variant B2L using anti B2L antibody generated as described in Example 3. Without wishing to be limited by a single hypothesis, the difference in sizes between lanes 4 and 5 is derived from the fact that lane 4 corresponds to the secreted protein and therefore it is probably glycosylated while lane 5 corresponds to the intracellular protein. Regarding the upper band, it is only seen with the rabbit polyclonal antibodies, so it could have a number of causes (which were not further examined), including non-specific interactions (for example due to excessive loading of the lane and/or presence of excessive amount of antibody).

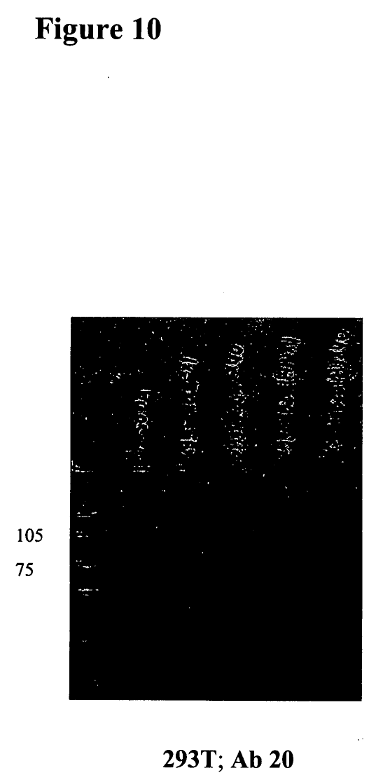

The secretion of the B2L protein following large scale transient transfection into 293T cells is demonstrated in FIG. 10, showing western blotting using Ab-20. 1-fold, or 40-fold TCA concentration of the supernatants of the B2L transfected cells or of the mock control were analyzed, as indicated. It was estimated that 87% of total B2L is secreted in 293T cells.

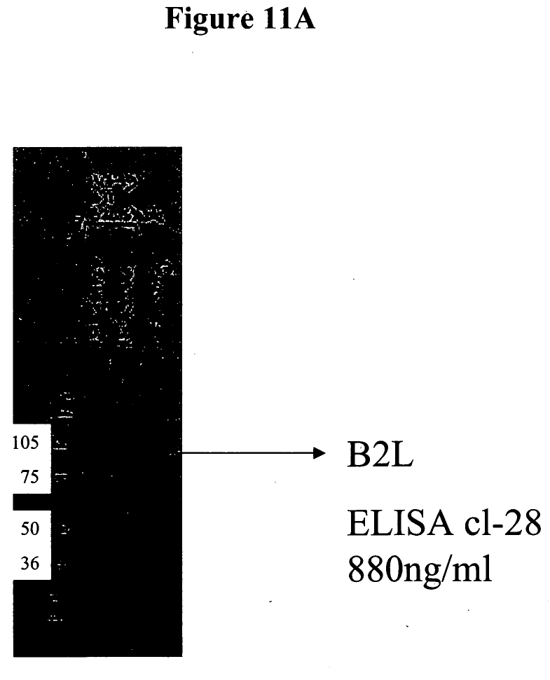

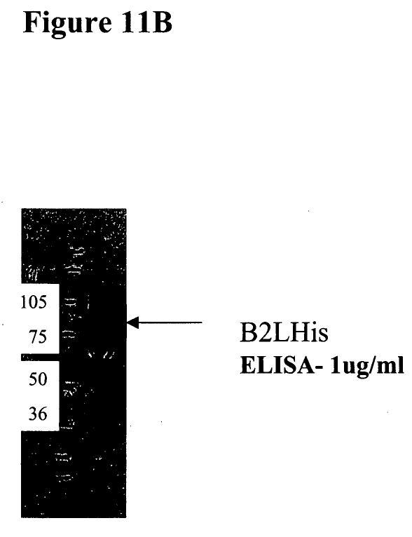

Stable expression of B2L with and without His tag was demonstrated following stable transfection of CHO or 293T cells, respectively, and is shown in FIGS. 11a and 11b, respectively. His-tag was always used at the C-terminus. Both western blots were performed using Ab-20 antibody. ELISA quantitation of the recombinant protein was carried out and showed expression levels of ˜1 μg/ml.

Interestingly, expression analysis of stably expressed B2S variant showed that it was expressed to a significantly lower extent than the B2L variant (data not shown).

Without wishing to be limited by a single hypothesis, it is possible that stable transfection with the B2S variant was less efficient, and/or that this variant may have caused the cells to die and/or show significantly reduced growth.

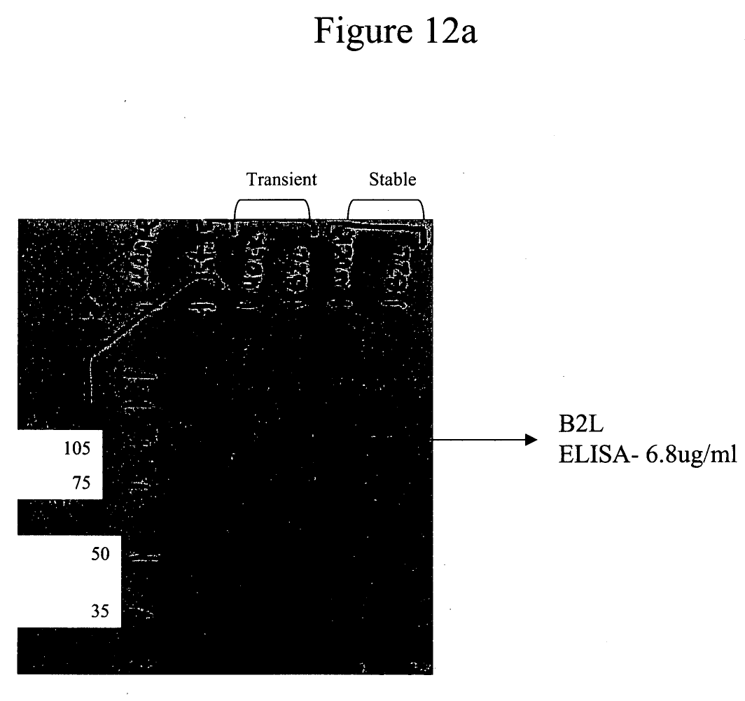

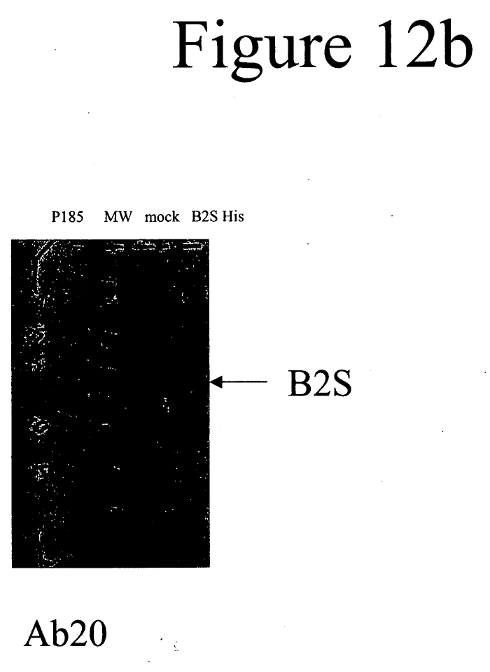

FIG. 12a shows a Western blot analysis of stable expression (with pIRESpuro vector) in 293T pools of B2L, using Ab 20. FIG. 12a clearly shows that the B2L variant was stably expressed at a high level in 293T pools. An ELISA assay showed that the level of B2L in the conditioned medium was 6.8 μg/ml. A Western blot analysis was also performed for stably transfected B2S-his in 293T, using Ab 20. Similar results were obtained (FIG. 12b).

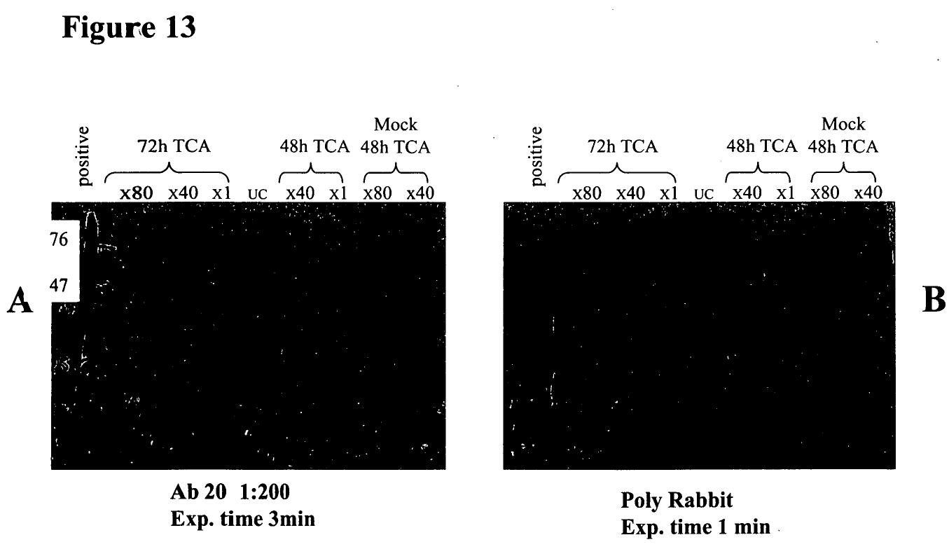

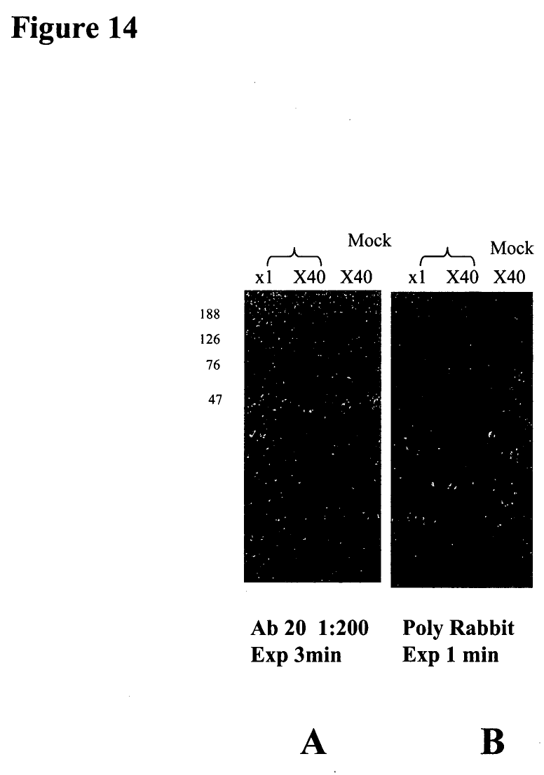

The time course of transient expression of B2S variant in COS7 cells was analyzed in a small scale transfection assay. As is demonstrated in FIGS. 13a-b, the expression was detected 48 and 72 hours post transfection. FIG. 13a shows the western blot analysis of B2S transient expression using Ab-20 antibody, while FIG. 13b shows western blot analysis using the specific polyclonal rabbit anti-B2S antibody. The first lane in the blots in FIGS. 13a-b is a positive control, which is a bacterially expressed B2S. The lane marked as “UC” is an unconcentrated conditioned medium. 1-fold, 40-fold and 80-fold TCA concentrations of the transfected cells or the mock control cell extracts are shown, as indicated. Expression is clearly detected 48 hrs and 72 hrs post transfection. A large scale transient expression of B2S in COS7 cells was analysed and the results are shown in FIGS. 14a-b. FIG. 14a shows the Western blot analysis of B2S transient expression using Ab-20 antibody, while FIG. 14b shows Western blot analysis using the specific polyclonal rabbit anti-B2S antibody. 1-fold or 40-fold TCA concentrated lysates of the transfected cells or the mock are shown, as indicated, although clear bands are only shown for 40-fold concentrated medium.

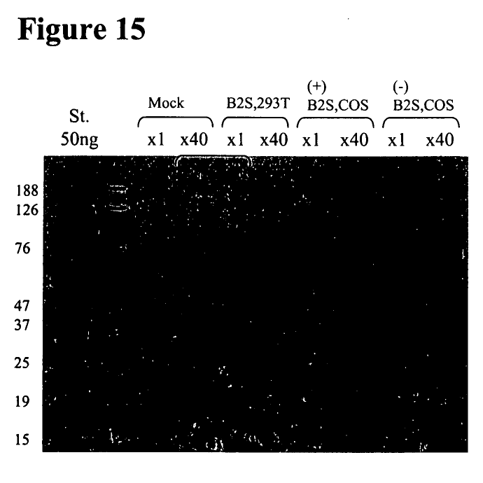

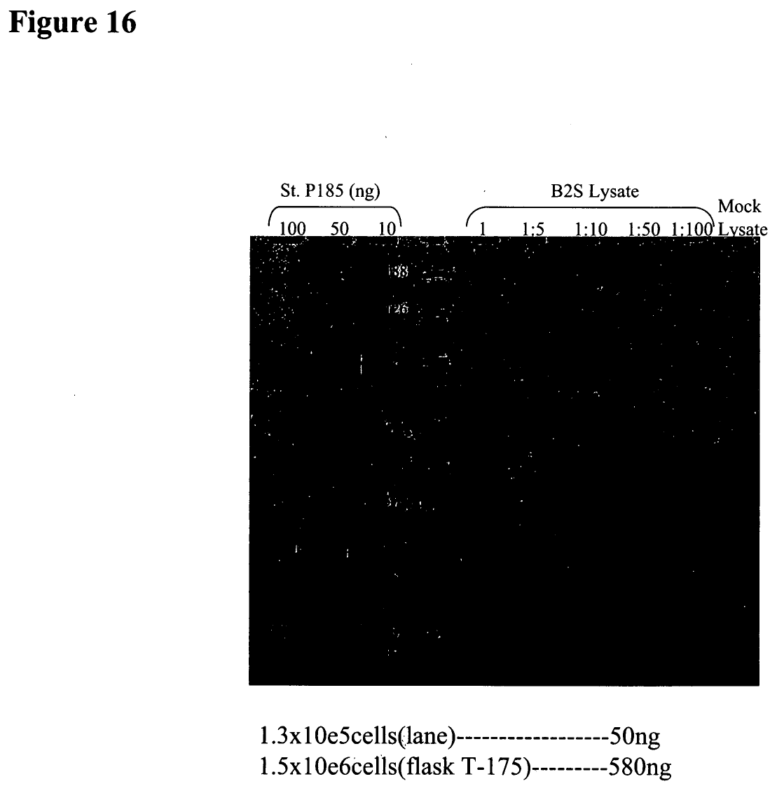

The transient expression of the B2S variant in supernatants of the 293T cells or COS7 cells as well as transient intracellular expression of B2S in COS7 cells was further analyzed, and the results are shown in FIGS. 15 and 16, respectively. The expression of B2S variant in the supernatant from different transient transfection experiments was analysed both in 293T and COS7 cells. FIG. 15 shows a Western blot, using the Ab-20 antibody, which demonstrates the secretion of the recombinant B2S protein into the supernatants of both cell lines. 1-fold and 40 fold TCA concentration of the supernatant of the B2S transfected cells or the mock control are shown, as indicated. The “mock” features mock-transfected COS7 cells. The cells were transfected also using the pAdVantage vector (Promega); however, the use of this vector did not appear to significantly change the levels of expression as is indicated by comparing lanes 7-8 to lanes 9-10 in FIG. 15.

The intracellular expression of the B2S variant was demonstrated following transient transfection of the COS7 cells, and the results are shown in FIG. 16. The expression of B2S in cell lysates with or without dilution is shown, as compared to mock lysate and to standard P185 control [i.e., extracellular portion of ErbB-2 gene product, sp185-Her2 from Bender MedSystems GmbH (Austria)], as indicated. The undiluted lysate sample in lane 5 was estimated to contain approximately 50 ng of the B2S variant protein. These results show that a significant portion of the protein was not secreted.

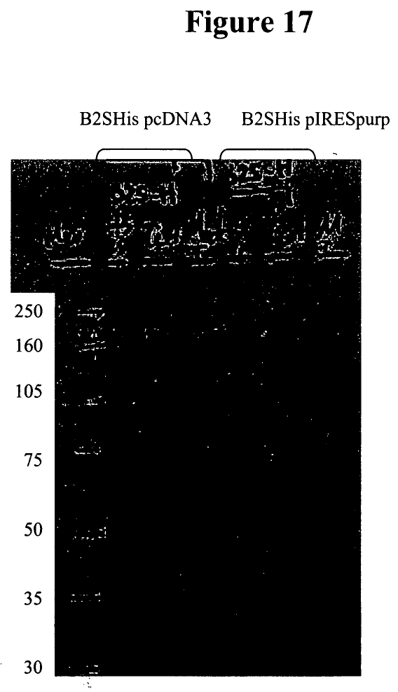

FIG. 17 shows western blot analysis of transiently expressed recombinant His-tagged B2S in 293T cells, using Ab-20 antibody. Both pcDNA3 and pIRES puro3 expression vectors encoding the His-B2S were transiently transfected into the 293T cells, and the specific expression is shown in FIG. 17, as compared to mock.

### Example 4F

B2L Purification Using Immunoaffinity

Proteins expressed in 293T were purified by immunoaffinity as described in greater detail below. The purifying antibody used was Herceptin.

Preparing a herceptin column—Coupling the Herceptin antibodies to CNBr activated Media was effected using the buffers listed in Table 12, below.

TABLE 12

| Buffer | Content |
|---|---|
| Medium washing | 1 mM HCl; Coupling Buffer: 0.25M NaHCO₃ pH |
| buffer | 9.0 + 0.5M NaCl |
| Blocking Buffer | 0.1M Tris-HCl pH 8.0 |
| Acidic wash buffer | 0.1M Acetate buffer pH 4 + 0.5M NaCl |
| Alkaline wash buffer | 0.1M Tris-HCl pH 8.0 + 0.5M NaCl |

Other materials included Herceptin: powder manufactured by Roche, reconstituted in ddH₂O 21 mg/ml and stored at −20° C.; CNBr activated Sepharose 4 FF (cat#17-0981-01).

The antibody solution to be coupled was mixed or dialyzed with coupling buffer. Various dilutions of samples were saved for analysis (sample #I-control 5 μl). The antibody:bead ratio used was 5:1 (mg/ml) or lower. In the present example the antibody amount added was 42 mg, antibody solution volume was 2.0 ml, and coupling buffer volume was 10 ml.

The matrix powder was suspended in 1 matrix volume of cold 1 mM HCl, mixed for 5 min and the liquid was removed (each 10 ml volume of matrix gel used 3 g dry powder). The washing step was repeated 5 times. The final matrix-volume was determined following the centrifugation of the suspension, and was estimated as 3.0 gr.

The washed beads were mixed with ±2 volume of coupling buffer.

The antibody solution was mixed with the matrix. A sample of the supernatant solution was kept for analysis (sample #2-Loaded 8.5 μl). The reaction was performed for 2-4 hrs at R.T (room temperature) or overnight at 4° C., after which all of the uncoupled solution was drained from the column. A sample from the flow-through was kept for the analysis. (sample #3-Uncoupled 8.5 μl). The unbound ligand was washed away with at least 7 volumes of coupling buffer.

To block the non-reacted groups, the drained medium was transferred to 0.1 M Tris HCl pH 8.0 or 1 M ethanolamine pH 8.0 for 2 hrs. Next, the matrix was washed as follows: 1 bed vol. of acidic wash buffer than 1 bed vol. of alkaline wash buffer. This cycle was repeated 4 times. Some of the beads were saved for analysis (sample #4-coupled 25 μl). The beads were stored in PBS containing 0.05% Azide.

Purification of B2L and Mock by Immunoaffinity Columns—B2L variant and mock-transfected cell material were subjected to immunoaffinity purification using a two column protocol as follows. Buffers are listed in Tables 13-14 below, which desribe the buffer content for the herceptin column and Mono Q column, respectively.

TABLE 13
First column

| Buffer | Content |
|---|---|
| Equilibration buffer | PBS (Biological industries) |
| Wash buffer: | PBS 0.5M NaCl |
| Elution buffer | glycine-HCl 100 mM, pH 2.8 TRIS 1M, pH 8.5 |

B2L and Mock mammalian supernatants were thawed and loaded on the Herceptin column described above using FPLC at a flow rate of 0.7 ml/min at 4C. The beads were washed with the wash buffer until OD was 0.01 mAu, followed by washing with PBS. Elution was preformed with 0.1M glycine pH 3.0 buffer, after which the pH value was adjusted with 10% Tris 1M pH 8.5 to pH 7.0.

TABLE 14

| Buffer | Content |
|---|---|
| Equilibration and wash buffer | 20 mM phosphate buffer pH 7.5 |
| Elution buffer | 20 mM phosphate buffer pH 7.5 with |
| | 1M NaCl |

The sample was diluted in water until the measured conductivity reached 3 mS/cm (which is the conductivity needed for binding to the Mono Q column resin, measured in milli Siemens units). The sample was loaded with a flow rate of 1 ml/min, after which B2L protein was eluted in a salt gradient 15 cv with a flow of 0.5 ml/min.

The purified proteins were then examined by gel electrophoresis, followed by Coomassie staining (described in greater detail below), which revealed highly purified proteins with only one major band at the expected molecular weight.

### Example 4G

B2L Purification by Conventional Chromatography

Proteins expressed exogenously in 293T cells were purified by conventional chromatography as described in greater detail below.

The procedure was effected as follows. First, total protein extraction was performed. A suspension of cell medium was centrifuged in 1500×g/10 min/4° C. using SLA-3000 Rotor, following the filtration of the superratant using 0.221 μm filter [Millipore filters, 0.22 μm (Cat# SCGP U 11 RE)]. Aliquots were stored at −70° C. Prior to use, the protein sample was thawed in a water bath, and then diluted to 3.6 mS (milli Siemens). Sample pH was adjusted to 5.2 with 0.5M citric acid, and the sample was filtered through a 0.22 μm filter. All buffers, eluants and samples were kept on ice throughout the process.

B2L was purified using a first step of a cation exchange column a second step of a Con-A column and a third step of a Mono Q column. Buffers used in the two purification steps were as listed in Table 15, below.

TABLE 15

| Buffer | Content |
|---|---|
| Buffer A: | 20 mM citrate-phosphate buffer, pH 5.2 |
| Buffer B | buffer A + 1M NaCl |
| Buffer C | 20 mM Tris.HCl, 0.5M NaCl, 1 mM MnCl2, 1 mM |
| | CaCl2 pH 7.4 |
| Buffer D | Buffer C without NaCl |
| Buffer E | 0.5M methyl-alpha-D-mannoside, 20 mM Tris- |
| | HCl pH 7.4 |
| Buffer F | 20 mM phosphate buffer pH 8.0 |
| Buffer G | 20 mM phosphate buffer + 1M NaCl pH 7.2 |

The protein sample was loaded on FPLC on 10 ml SP-Sepharose beads (cation exchanger column material), previously loaded with 3 CV (column volumes) of buffer B and equilibrated with 15 CV of buffer A, with a flow rate of 10 ml/min. The column was then washed with buffer A at the rate of 1 ml/min, until O.D280ₙₘ was less than 0.001. This process was followed by elution with a step gradient of buffer B 0-100%, as follows: (Flow=0.5 m/min; 1 ml fraction) 30% B 10CV, as the B2L protein elutes in the step of 30% buffer B, followed by washing with 100% Buffer B 10CV to remove any contaminants. SDS-PAGE analysis was done to detect the fractions containing the B2L Variant protein. Variant containing fractions were combined and stored overnight at 4° C.

The protein sample was then loaded on 1 ml Con-A sepharose beads previously equilibrated with 15 CV of buffer C (flow during equilibration was 1 ml/min), followed by incubation overnight in a rocking tube at 4° C. After incubation the beads were packed in 1 ml column, which was washed with buffer C at the rate of 1 ml/min, until O.D280ₙₘ was less than 0.001. The column was then washed with buffer D, following elution with step gradient of buffer E100%, as follows: (Flow=0.2 ml/min; 1 ml fraction), 100% E/10CV, which was maintained if necessary until the B2L protein peak was reduced to less than 10% of its maximal height. SDS-PAGE analysis was done to detect the fractions containing the B2L Variant protein. Variant containing fractions were combined and stored overnight at 4° C.

The protein sample was then loaded on FPLC on 1 ml Mono Q beads previously loaded with 3 CV of buffer G and equilibrated with 15 CV of buffer F, Flow=1 ml/min. The beads were then washed with buffer F at the rate of 1 ml/min, until O.D280ₙₘ is less then 0.001, following elution with a gradient of buffer A 0-100% 15 CV, as follows: (Flow=0.2 m/min; 1 ml fraction).

SDS-PAGE analysis was done to detect the fractions containing the B2L variant protein.

Variant containing fractions were combined and stored overnight at 4° C. or at −70° C.

FIGS. 18a-b show the results of the above-described immunoaffinity purification of B2L variant protein, expressed in 293T cells (stable pool of 293T cells), as a Coomassie stained gel and a Western blot (using antibody Ab 20) respectively (protocols are described below with regard to Example 5).

As can be seen, the supernatant had a clear single band on the Western blot but many bands were present in the Coomassie stained gel. Elution pool I provided sufficient clean protein to be visible on the Coomassie stained gel as a single band, with a very strong signal on the Western blot. Elution pool II provided a weaker signal.

### Example 5

### Recognition of ErbB-2 B2L and B2S Variants by Specific Anti ErbB-2 Antibodies

Recognition of ErbB-2 variants was effected by Western blotting.

Materials and Experimental Procedures

Antibodies—anti-B2L and anti-B2S antibodies were prepared as described in Example 3 above. Monoclonal antibody, Ab-20, recognizing the extracellular portion of human ErbB-2 was from Neo Markers (Clone Designation is L87+2ERB19).

Western blot analysis—SDS-PAGE was performed as follows: The proteins (either non-purified bacterial preparations for the Western blot, or purified preparations as described with regard to Example 4) were resuspended in 30 μl 1×SDS-sample buffer containing 50 mM DTT (crude preparation). Following warming for 10 min and subsequent centrifugation, samples were loaded on Nu-PAGE BIS TRIS gel buffer system (12%, Invitrogen).

Following electrophoresis (150V, 90 minutes), gels were washed with cold transfer buffer for 15 min, followed by transfer with Nitrocellulose membranes for 60 min at 30 V using Invitrogen's transfer buffer and X-Cell II blot module. Following transfer, blots were blocked with PBST-5% skim milk (0.05% Tween-20 in PBS buffer) for at least 60 min at room temperature or overnight at 4° C. Following blocking, blots were incubated with antibodies (either the previously described anti-splice variant antibodies or a commercially available Ab 20 antibody) at ˜1 [symbol] g/ml for 1-3 hrs, washed with 0.05% Tween 20 in PBS, incubated with respective peroxidase-conjugated antibodies, washed with PBS-Tween-20 solution, followed by ECL (either Super Signal Chemiluminescence Substrate, Pierce, or EZ-ECL, Biological Industries) including exposure to X-ray film.

Coomassie staining—see Example 4.

Results

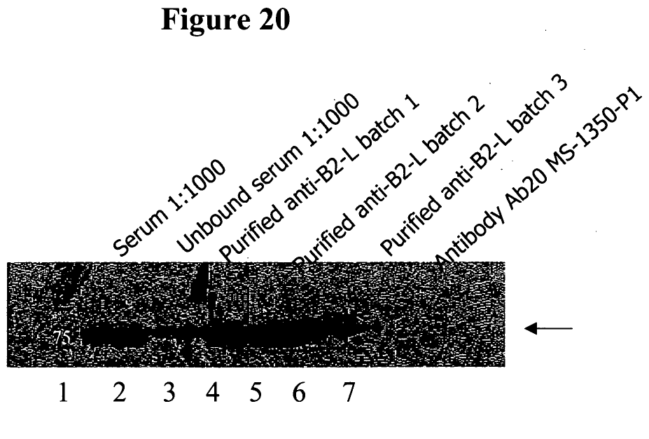

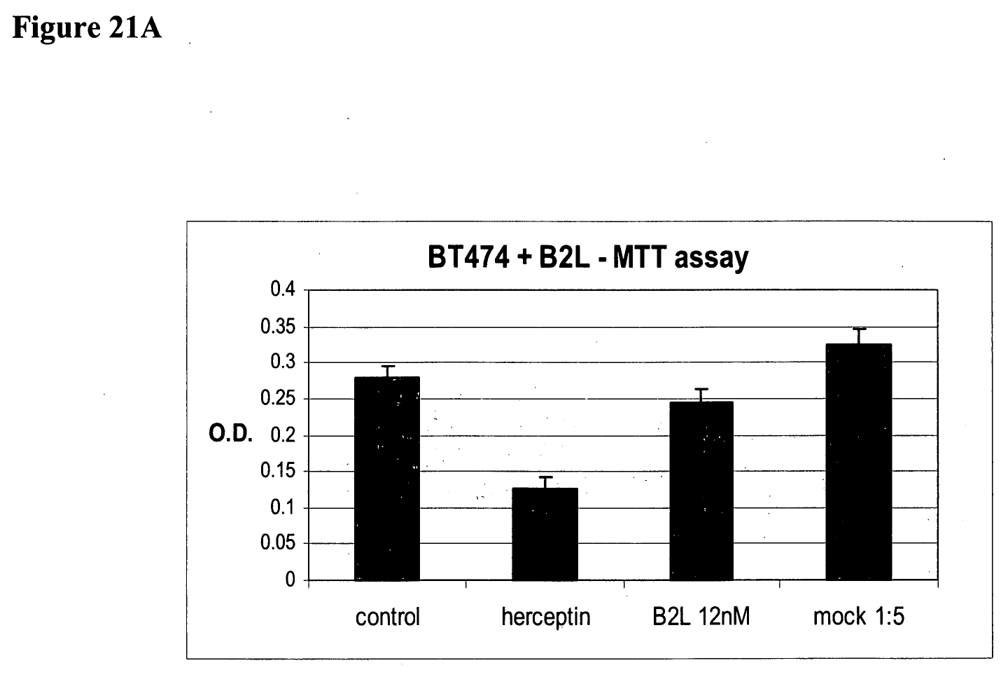

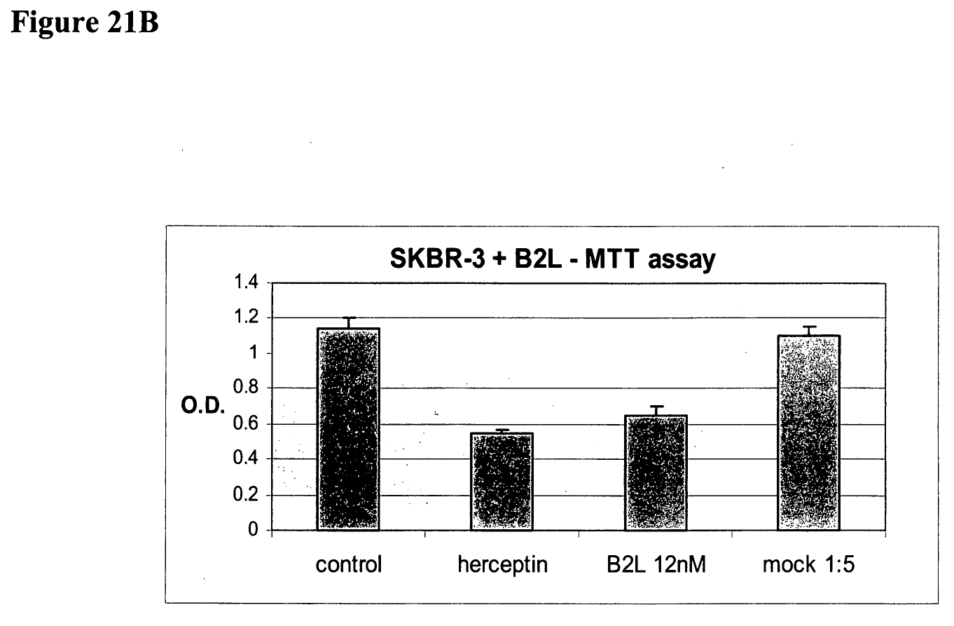

The results are shown in FIGS. 19 and 20. Briefly, FIG. 19 shows the performance of purified anti-B2S antibodies, in comparison to herceptin (anti-ErbB-2 antibody). FIG. 20 shows the purified B2L antibody performance. The anti-B2S and B2L antibodies clearly demonstrate specific binding with one significant band. Molecular weight markers are shown at the far left.

### Example 6

### Functional Activity Assays of the ErbB-2 B2L Variant

The functional activity of the long (B2L) ErbB-2 variant according of the present invention was tested with cell-based assays testing proliferation.

Cells—The following breast carcinoma-derived cell lines were used: BT-474 (ATCC HTB-20, Manassas, Va.) and T-47D (ATCC HTB-133), which were propagated in Dulbecco's modified Eagle's medium (DMEM, Biological Industries, Beit Haemek, Israel) containing 10% dialyzed fetal bovine serum (dFBS, Hyclone); SK-BR-3 (ATCC HTB-30) and MCF-7 (ATCC HTB-22), which were propagated in McCoy's 5A and MEM-eagle media (Biological Industries, Beit Haemek, Israel), respectively, supplemented with 10% dFBS. Cells were grown at 37° C. in an atmosphere containing 5% CO₂. Note, SKBR3 and BT474 overexpress wild-type ErbB2, while MCF-7 and T-47D have low expression levels of wild-type ErbB2 (see for example Moasser et al., Cancer research 61, 7184-7188, 2001).

MTT assay—Cells were seeded into 96-well microtiter plates at a density of 10⁴ cells per well and allowed to adhere overnight. For SK-BR-3 and BT-474, the media were then replaced with low-serum media (1% dFBS) containing the following additions: 20 μg/ml Herceptin (Trastuzumab, Roche, Switzerland), which is the humanized anti-ErbB-2 antibody (Roch), 20 μg/ml of a non-specific antibody as a negative control, or conditioned medium containing secreted B2L protein of HEK293 cells transfected with a B2L-pIRES-puro DNA construct. Conditioned medium that was collected from HEK293 cells transfected with a mock-construct was added as a negative control at the same dilutions. Cell proliferation was monitored for 6 days using the MTT reagent (Thiazolyl blue, Sigma). In brief, 10 μl of MTT reagent (5 mg/ml) were added to 100 μl of medium in the well. Following 4 hrs of incubation the medium was aspirated and 100 μl of 100% DMSO were added to each well. After 2 hrs, the optical density (O.D.) of each well was determined using a microplate reader set to 490 nm.

For MCF-7 and T-47D, after the cells were allowed to adhere overnight, the medium was removed and replaced with medium containing 0.1% dFBS. Following a 6 hrs period of serum starvation, the cells were treated with different dilutions of conditioned medium containing B2L or mock for 1 hr, followed by the addition of 1 nM Heregulin-1 (HRG1-131 EGF domain, R&D Systems, Minneapolis, Minn.). Cells were incubated for 3 days at 37° C. and their relative growth was measured using MTT reagent (described above).

Results

The results of the MTT experiments are shown in FIGS. 21a-b. As expected, herceptin inhibited the proliferation of BT474 and SKBR3 (see FIGS. 21a-b, respectively). Such effective inhibition was exprected as these cell-lines overexpress ErbB-2 and depend on it for their growth. The conditioned media containing the B2L protein (12 nM) showed inhibitory activity when added to these cells, whereas the mock media at the appropriate dilution did not show such inhibitory activity. The effect was more strongly seen in FIG. 21b with the SKBR3 cell line, in which the degree of inhibition was similar to that seen with Herceptin. Again, mock medium had no effect on cell growth.

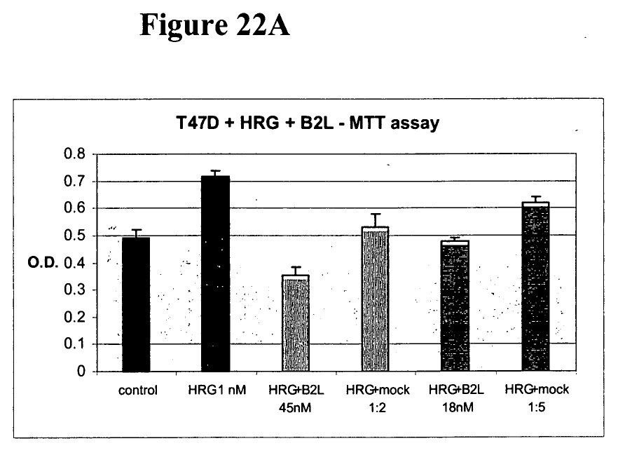

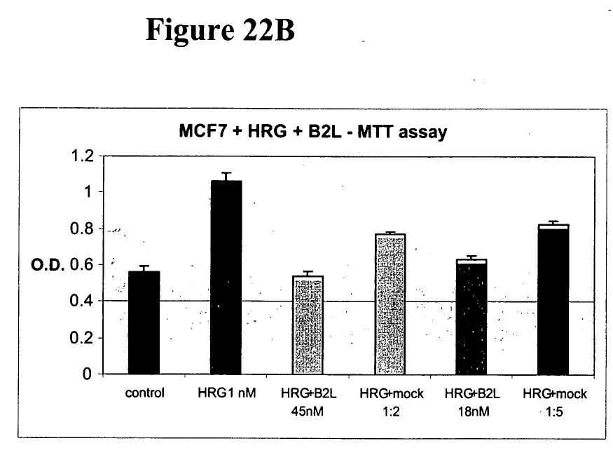

FIGS. 22a-b shows a ligand-dependent increase in proliferation following the addition of Heregulin-1 (HRG) to T47D and MCF7 cell lines (FIGS. 22a and 22b, respectively). As is evident, addition of conditioned medium containing the B2L protein had inhibitory activity on the proliferation of these cells, relative to the mock media at the appropriate dilution. It should be noted that the mock-transfected media (media from mock-transfected cells) had a small inhibitory effect on HRG-induced proliferation by itself, but this effect was much smaller than the inhibitory effect seen with B2L and HRG together. This inhibitory effect could be attributed to an unknown inhibitory factor released from 293T cells.

Altogether, these results suggest that B2L has an inhibitory activity on the proliferation of ErbB-2-overexpressing cells and Heregulin-induced cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An isolated polynucleotide comprising a nucleic acid sequence encoding at least an active portion of an ErbB-2 polypeptide including an amino acid sequence being at least 70% homologous to SEQ ID NO:5, as determined using the BlastP software of the National Center of Biotechnology Information (NCBI) using default parameters.

2. The isolated polynucleotide of claim 1, wherein said ErbB-2 polypeptide is as set forth in SEQ ID NO: 2, 5 or 14.

3. The isolated polynucleotide of claim 1, wherein said nucleic acid sequence is as set forth in SEQ ID NO. 1, 7 or 13.

4. The isolated polynucleotide of claim 1, wherein said active portion of said polypeptide is as set forth in SEQ ID NO:5.

5. The isolated polynucleotide of claim 4, wherein said active portion of said polypeptide is encoded by nucleotide coordinates 2097-2320 of SEQ ID NO:1.

6. An isolated polynucleotide comprising a nucleic acid sequence encoding at least an active portion of an ErbB-2 polypeptide including an amino acid sequence being at least 69% homologous to SEQ ID NO:6, as determined using the BlastP software of the National Center of Biotechnology Information (NCBI) using default parameters.

7. The isolated polynucleotide of claim 6, wherein said ErbB-2 polypeptide is as set forth in SEQ ID NO:4, 6 or 16.

8. The isolated polynucleotide of claim 6, wherein said nucleic acid sequence is as set forth in SEQ ID NO. 3, 8 or 15.

9. The isolated polynucleotide of claim 6, wherein said active portion of said polypeptide is as set forth in SEQ ID NO:6.

10. The isolated polynucleotide of claim 9, wherein said active portion of said polypeptide is encoded by nucleotide coordinates 1664-1944 of SEQ ID NO:3.

11. A nucleic acid construct comprising the isolated polynucleotide of claim 1.

12. The nucleic acid construct of claim 11, further comprising a promoter for regulating transcription of the isolated polynucleotide in sense or antisense orientation.

13. The nucleic acid construct of claim 11, further comprising a positive and a negative selection marker for selecting for homologous recombination events.

14. A host cell comprising the nucleic acid construct of claim 11.

15. A nucleic acid construct comprising the isolated polynucleotide of claim 6.

16. The nucleic acid construct of claim 15, further comprising a promoter for regulating transcription of the isolated polynucleotide in sense or antisense orientation.

17. The nucleic acid construct of claim 15, further comprising a positive and a negative selection markers for selecting for homologous recombination events.

18. A host cell comprising the nucleic acid construct of claim 15.

19. An isolated polypeptide as set forth in SEQ ID NO:2, 4 or 10 or an active portion thereof.

20. An isolated polypeptide comprising an ErbB-2 polypeptide including an amino acid sequence being at least 71% homologous to SEQ ID NO:5, as determined using the BlastP software of the National Center of Biotechnology information (NCBI) using default parameters or an active portion thereof.

21. The isolated polypeptide of claim 20, wherein said amino acid sequence is as set forth in SEQ ID NO:2, 5 or 14.

22. The isolated polypeptide of claim 20, wherein said active portion of the polypeptide is as set forth is SEQ ID NO:5.

23. An isolated polypeptide comprising an ErbB-2 polypeptide including an amino acid sequence being at least 69% homologous to SEQ ID NO:6, as determined using the BlastP software of the National Center of Biotechnology Information (NCBI) using default parameters.

24. An isolated polypeptide of ErbB-2 variant I (SEQ ID NO: 2), comprising a first amino acid sequence being at least 90% homologous to amino acids 1-648 of wild type ErbB-2 (GenBank Accession No: gi: AAA75493), which also encompasses amino acids 1-648 of the sequence as set forth in SEQ ID NO:2, and a second amino acid sequence being at least about 70%, optionally at least about 80%, preferably at least about 85%, more preferably at least about 90% and most preferably at least about 95% homologous to a polypeptide having the sequence RLAWTPGCTLHCPSLPHWMLGGHCCREGTP (SEQ ID NO: 5), wherein said first and said second amino acid sequences are contiguous and in a sequential order.

25. An isolated polypeptide of a tail of ErbB-2 variant I (SEQ ID NO: 2), comprising a polypeptide having the sequence RLAWTPGCTLHCPSLPHWMLGGHCCREGTP (SEQ ID NO: 5).

26. An isolated polypeptide of a tail of ErbB-2 variant I (SEQ ID NO: 2), comprising a polypeptide being at least 70%, optionally at least about 80%, preferably at least about 85%, more preferably at least about 90% and most preferably at least about 95% homologous to the sequence RLAWTPGCTLHCPSLPHWMLGGHCCREGTP (SEQ ID NO: 5).

27. An isolated polypeptide of a bridge portion of SEQ ID NO:2, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise AR, having a structure as follows (numbering according to SEQ ID NO:2): a sequence starting from any of amino acid number 648-x to 648; and ending at any of amino acid numbers 649+((n−2)−x), in which x varies from 0 to n−2, with the proviso that the value ((n−2)−x) is not allowed to be larger than 29.

28. An isolated polypeptide of the bridge portion of SEQ ID NO:2, comprising a polypeptide being at least 70%, optionally at least about 80%, preferably at least about 85%, more preferably at least about 90% and most preferably at least about 95% homologous to polypeptide of claim 27.

29. An isolated polypeptide of ErbB-2 variant II (SEQ ID NO: 4), comprising a first amino acid sequence being at least 90% homologous to amino acids 1-504 of wild type ErbB-2 (GenBank Accession No: gi: AAA75493), which also encompasses amino acids 1-504 of the sequence as set forth in SEQ ID NO: 4, and a second amino acid sequence being at least about 70%, optionally at least about 80%, preferably at least about 85%, more preferably at least about 90% and most preferably at least about 95% homologous to a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6), wherein said first and said second amino acid sequences are contiguous and in a sequential order.

30. An isolated polypeptide of a tail of ErbB-2 variant II (SEQ ID NO: 4), comprising a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6).

31. An isolated polypeptide of a tail of ErbB-2 variant II (SEQ ID NO: 4), comprising a polypeptide being at least 70%, optionally at least about 80%, preferably at least about 85%, more preferably at least about 90% and most preferably at least about 95% homologous to the sequence GKTGSPVCALPICQHTAVPRGPWQQRSWTCADCPSLCTLLDSAQLWLAWPL GMASLAGSYLPWHPSLPLCF (SEQ ID NO: 6).

32. An isolated polypeptide of a bridge portion of SEQ ID NO:4, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise CG, having a structure as follows (numbering according to SEQ ID NO:4): a sequence starting from any of amino acid number 504-x to 504; and ending at any of amino acid numbers 505+((n−2)−x), in which x varies from 0 to n−2, with the proviso that the value ((n−2)−x) is not allowed to be larger than 70.

33. An isolated polypeptide of a bridge portion of SEQ ID NO:4, comprising a polypeptide being at least 70%, optionally at least about 80%, preferably at least about 85%, more preferably at least about 90% and most preferably at least about 95% homologous to polypeptide of claim 32.

34. An isolated polypeptide of ErbB-2 variant III (SEQ ID NO: 10), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 10, an amino acid sequence being at least about 70%, optionally at least about 80%, preferably at least about 85%, more preferably at least about 90% and most preferably at least about 95% homologous to a polypeptide having the sequence VSLCQQAGVQWYDLGSLQPLPPGFKQFSCLSLLSSWDYR (SEQ ID NO: 11), and a second amino acid sequence being at least 90% homologous to amino acids 384-1255 of wild type ErbB-2 (GenBank Accession No: gi: AAA75493), which also encompasses amino acids 423-1294 of the sequence as set forth in SEQ ID NO: 10, wherein said first amino acid is contiguous to said bridging polypeptide and said second amino acid sequence is contiguous to said bridging polypeptide, and wherein said first amino acid, said bridging polypeptide and said second amino acid sequence are in a sequential order.

35. An isolated polypeptide encoding for an edge portion of ErbB-2 variant III (SEQ ID NO: 10), comprising a polypeptide having the sequence VSLCQQAGVQWYDLGSLQPLPPGFKQFSCLSLLSSWDYR (SEQ ID NO: 11).

36. An isolated polypeptide encoding for a unique edge portion of SEQ ID NO:10, comprising a polypeptide being at least 70%, optionally at least about 80%, preferably at least about 85%, more preferably at least about 90% and most preferably at least about 95% homologous to the sequence VSLCQQAGVQWYDLGSLQPLPPGFKQFSCLSLLSSWDYR (SEQ ID NO: 11).

37. An isolated polypeptide encoding for a bridge portion of SEQ ID NO: 10, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino aids in length, wherein at least two amino acids comprise GV, having a structure as follows (numbering according to SEQ ID NO:10): a sequence starting from any of amino acid number 383-x to 383; and ending at any of amino acid numbers 384+((n−2)−x), in which x varies from 0 to n−2.

38. An isolated polypeptide encoding for bridge portion of a SEQ ID NO: 10, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RD, having a structure as follows (numbering according to SEQ ID NO:10): a sequence starting from any of amino acid number 421-x to 421; and ending at any of amino acid numbers 422+((n−2)−x), in which x varies from 0 to n−2.

39. An isolated polypeptide, comprising a polypeptide being at least 70%, optionally at least about 80%, preferably at least about 85%, more preferably at least about 90% and most preferably at least about 95% homologous to the any of the polypeptides of claim 37.

40. An isolated polypeptide of ErbB-2 variant IV (SEQ ID NO: 14), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 14, an amino acid sequence being at least about 70%, optionally at least about 80%, preferably at least about 85%, more preferably at least about 90% and most preferably at least about 95% homologous to a polypeptide having the sequence VSLCQQAGVQWYDLGSLQPLPPGFKQFSCLSLLSSWDYR (SEQ ID NO: 11), a second amino acid sequence being at least 90% homologous to amino acids 384-648 of wild type ErbB-2 (GenBank Accession No: gi: AAA75493), which also encompasses amino acids 423-687 of the sequence as set forth in SEQ ID NO: 14, followed by an amino acid sequence tail being at least about 70%, optionally at least about 80%, preferably at least about 85%, more preferably at least about 90% and most preferably at least about 95% homologous to a polypeptide having the sequence RLAWTP GCTLHCPSLPHWMLGGHCCREGTP (SEQ ID NO: 5), wherein said first amino acid is contiguous to said bridging polypeptide and said second amino acid sequence is contiguous to said bridging polypeptide, and wherein said first amino acid, said bridging polypeptide, said second amino acid and said amino acid sequence tail are in a sequential order.

41. An isolated polypeptide of ErbB-2 variant V (SEQ ID NO: 16), comprising a first amino acid sequence being at least 90% homologous to amino acids 1-383 of wild type ErbB-2 (GenBank Accession No: gi: AAA75493), which also encompasses amino acids 1-383 of the sequence as set forth in SEQ ID NO: 16, an amino acid sequence being at least about 70%, optionally at least about 80%, preferably at least about 85%, more preferably at least about 90% and most preferably at least about 95% homologous to a polypeptide having the sequence VSLCQQAGVQWYDLGSLQPLPPGFKQFSCLSLLSSWDYR (SEQ ID NO: 11), a second amino acid sequence being at least 90% homologous to amino acids 384-804 of wild type ErbB-2 (GenBank Accession No: gi: AAA75493), which also encompasses amino acids 423-543 of the sequence as set forth in SEQ ID NO: 16, followed by an amino acid sequence tail being at least about 70%, optionally at least about 80%, preferably at least about 85%, more preferably at least about 90% and most preferably at least about 95% homologous to a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6), wherein said first amino acid is contiguous to said bridging polypeptide and said second amino acid sequence is contiguous to said bridging polypeptide, and wherein said first amino acid, said bridging polypeptide, said second amino acid and said amino acid sequence tail are in a sequential order.

42. An isolated polypeptide of ErbB-2 variant VI (SEQ ID NO: 26), comprising a first amino acid sequence being at least 90% homologous to amino acids 1-340 of wild type ErbB-2 (GenBank Accession No: gi: AAA75493), which also encompasses amino ac ids 1-340 of the sequence as set forth in SEQ ID NO:26, and a second amino acid sequence being at least about 70%, optionally at least about 80%, preferably at least about 85%, more preferably at least about 90% and most preferably at least about 95% homologous to a polypeptide having the sequence GTQPPTLPRSSQSSSKCLRLWKRSQVTYTSQHGRTACLTSASSRTCK (SEQ ID NO: 28), wherein said first and said second amino acid sequences are contiguous and in a sequential order.

43. An isolated polypeptide of a tail of ErbB-2 variant VI (SEQ ID NO: 26), comprising a polypeptide having the sequence GTQPPTLPRSSQSSSKCLRLWKRSQVTYTSQHGRTACLTSASSRTCK (SEQ ID NO: 28).

44. An isolated polypeptide of a tail of ErbB-2 variant VI (SEQ ID NO: 26), comprising a polypeptide being at least 70%, optionally at least about 80%, preferably at least about 85%, more preferably at least about 90% and most preferably at least about 95% homologous to the sequence GTQPPTLPRSSQSSSKCLRLWKRSQVTYTSQHGRTACLTSASSRTCK (SEQ ID NO: 28).

45. An isolated polypeptide encoding for a bridge portion of SEQ ID NO:26, comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RG, having a structure as follows (numbering according to SEQ ID NO:26): a sequence starting from any of amino acid number 340-x to 340; and ending at any of amino acid numbers 341+((n−2)−x), in which x varies from 0 to n−2, with the proviso that the value ((n−2)−x) is not allowed to be larger than 46.

46. An isolated polypeptide encoding for a bridge portion of SEQ ID NO:26, comprising a polypeptide being at least 70%, optionally at least about 80%, preferably at least about 85%, more preferably at least about 90% and most preferably at least about 95% homologous to any of the polypeptides of claim 45.

47. An isolated polynucleotide sequence encoding the polypeptides of ErbB-2 variant I of claim 24, or fragments thereof.

48. An isolated polynucleotide of ErbB-2 variant I of claim 34, wherein said ErbB-2 variant I polypeptide is as set forth in SEQ ID NO: 2 or 5.

49. An isolated polynucleotide of ErbB-2 variant I, wherein said nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 1.

50. The isolated polynucleotide of ErbB-2 variant I, wherein said nucleic acid sequence is as set forth in SEQ ID NO: 1.

51. An isolated nucleic acid sequence encoding the polypeptides of ErbB-2 variant II of claim 29, or fragments thereof.

52. An isolated polynucleotide of ErbB-2 variant II of claim 38, wherein said ErbB-2 variant II polypeptide is as set forth in SEQ ID NO: 4 or 6.

53. An isolated polynucleotide of ErbB-2 variant II, wherein said nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 3.

54. The isolated polynucleotide of ErbB-2 variant II, wherein said nucleic acid sequence is as set forth in SEQ ID NO: 3.

55. An isolated nucleic acid sequence encoding the polypeptides of ErbB-2 variant III of claim 34, or fragments thereof.

56. An isolated polynucleotide of ErbB-2 variant III of claim 55, wherein said ErbB-2 variant III polypeptide is as set forth in SEQ ID NO: 10 or 11.

57. An isolated polynucleotide of ErbB-2 variant III, wherein said nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 9.

58. The isolated polynucleotide of ErbB-2 variant III, wherein said nucleic acid sequence is as set forth in SEQ ID NO: 9.

59. An isolated nucleic acid sequence encoding the said polypeptides of ErbB-2 variant IV or fragments thereof.

60. An isolated polynucleotide of ErbB-2 variant IV of claim 40, wherein said ErbB-2 variant IV polypeptide is as set forth in SEQ ID NO: 14.

61. An isolated polynucleotide of ErbB-2 variant IV, wherein said nucleic acid sequence comprises a sequence being at least about 70%, optionally at least about 80%, preferably at least about 85%, more preferably at least about 90% and most preferably at least about 95% homologous to a nucleic acid sequence is as set forth in SEQ ID NO: 13.

62. The isolated polynucleotide of ErbB-2 variant IV, wherein said nucleic acid sequence is as set forth in SEQ ID NO: 13.

63. An isolated nucleic acid sequence encoding the polypeptides of ErbB-2 variant V of claim 41, or fragments thereof.

64. An isolated polynucleotide of ErbB-2 variant V of claim 63, wherein said ErbB-2 variant V polypeptide is as set forth in SEQ ID NO: 16, 6 or 11.

65. An isolated polynucleotide of ErbB-2 variant IV, wherein said nucleic acid sequence comprises a sequence being at bast about 70%, optionally at least about 80%, preferably at least about 85%, more preferably at least about 90% and most preferably at least about 95% homologous to a nucleic acid sequence is as set forth in SEQ ID NO: 15.

66. The isolated polynucleotide of ErbB-2 variant V, wherein said nucleic acid sequence is as set forth in SEQ ID NO: 15.

67. An isolated nucleic acid sequence encoding the polypeptides of ErbB-2 variant VI of claim 41, or fragments thereof.

68. An isolated polynucleotide of ErbB-2 variant VI of claim 67, wherein said ErbB-2 variant VI polypeptide is as set forth in SEQ ID NO: 26 or 28.

69. An isolated polynucleotide of ErbB-2 variant VI, wherein said nucleic acid sequence comprises a sequence being at least about 70%, optionally at bast about 80%, preferably at least about 85%, more preferably at least about 90% and most preferably at least about 95% homologous to a nucleic acid sequence is as set forth in SEQ ID NO: 25.

70. The isolated polynucleotide of ErbB-2 variant VI, wherein said nucleic acid sequence is as set forth in SEQ ID NO: 25.

71. An antibody or an antibody fragment being capable of specifically binding an ErbB-2 polypeptide including an amino acid sequence being at least 71% homologous to SEQ ID NO:5, as determined using the BlastP software of the National Center of Biotechnology Information (NCBI) using default parameters.

72. The antibody or the antibody fragment of claim 71, further comprising a functional moiety.

73. The antibody or the antibody fragment of claim 72, wherein said functional moiety is selected from the group consisting of a detectable moiety and a cytotoxic agent.

74. The antibody or antibody fragment of claim 71, wherein said polypeptide is as set forth in SEQ ID NO:2, 5 or 14.

75. A pharmaceutical composition comprising a therapeutically effective amount of the antibody or antibody fragment of claim 71, and a pharmaceutically acceptable carrier.

76. An antibody or an antibody fragment being capable of specifically binding an ErbB-2 polypeptide including an amino acid sequence being at least 69% homologous to SEQ ID NO:6, as determined using the BlastP software of the National Center of Biotechnology Information (NCBI) using default parameters.

77. The antibody or the antibody fragment of claim 76, further comprising a functional moiety.

78. The antibody or the antibody fragment of claim 77, wherein said functional moiety is selected from the group consisting of a detectable moiety and a cytotoxic drug.

79. The antibody or antibody fragment of claim 76, wherein said polypeptide is as set forth in SEQ ID NO:4, 6 or 16.

80. A pharmaceutical composition comprising a therapeutically effective amount of the antibody or antibody fragment of claim 76, and a pharmaceutically acceptable carrier.

81. An antibody or an antibody fragment being capable of specifically binding an ErbB-2 variant polypeptide of claim 24.

82. The antibody or the antibody fragment of claim 81, further comprising a functional moiety.

83. The antibody or the antibody fragment of claim 82, wherein said functional moiety is selected from the group consisting of a detectable moiety and a cytotoxic drug.

84. A pharmaceutical composition comprising a therapeutically effective amount of the antibody or antibody fragment of claim 81, and a pharmaceutically acceptable carrier.

85. An antibody or an antibody fragment being capable of specifically binding a polypeptide of claim 19.

86. The antibody or the antibody fragment of claim 85, further comprising a functional moiety.

87. The antibody or the antibody fragment of claim 86, wherein said functional moiety is selected from the group consisting of a detectable moiety and a cytotoxic drug.

88. A pharmaceutical composition comprising a therapeutically effective amount of the antibody or antibody fragment of claim 85, and a pharmaceutically acceptable carrier.

89. A display library comprising a plurality of display vehicles each displaying at least 6 consecutive amino acids derived from an ErbB-2 polypeptide including an amino acid sequence being at least 71% homologous to SEQ ID NO:5 as determined using the BlastP software of the National Center of Biotechnology Information (NCBI) using default parameters.

90. A display library comprising a plurality of display vehicles each displaying at least 6 consecutive amino acids derived from an ErbB-2 polypeptide including an amino acid sequence being at least 69% homologous to SEQ ID NO:6 as determined using the BlastP software of the National Center of Biotechnology Information (NCBI) using default parameters.

91. An oligonucleotide specifically hybridizable with a nucleic acid sequence encoding an ErbB-2 polypeptide including an amino acid being at least 71% homologous to SEQ ID NO:5, as determined using the BlastP software of the National Center of Biotechnology Information (NCBI) using default parameters.

92. The oligonucleotide of claim 91, wherein said nucleic acid sequence is as set forth in SEQ ID NO: 1, 7 or 13.

93. The oligonucleotide of claim 91, wherein said oligonucleotide is a single or double stranded.

94. The oligonucleotide of claim 91, wherein said oligonucleotide is at least 10 bases long.

95. The oligonucleotide of claim 91, wherein said oligonucleotide is hybridizable in either sense or antisense orientation.

96. An oligonucleotide specifically hybridizable with a nucleic acid sequence encoding an ErbB-2 polypeptide including an amino acid being at least 69% homologous to SEQ ID NO:6, as determined using the BlastP software of the National Center of Biotechnology Information (NCBI) using default parameters.

97. The oligonucleotide of claim 96, wherein said nucleic acid sequence is as set forth in SEQ ID NO:3, 8 or 15.

98. The oligonucleotide of claim 96, wherein said oligonucleotide is a single or double stranded.

99. The oligonucleotide of claim 96, wherein said oligonucleotide is at least 10 bases long.

100. The oligonucleotide of claim 96, wherein said oligonucleotide is hybridizable in either sense or antisense orientation.

101. The oligonucleotide of claim 96, attached to a solid support.

102. The oligonucleotide of claim 101, wherein said solid support is a bead.

103. The oligonucleotide of claim 102, wherein said solid support is a chip.

104. The oligonucleotide of claim 96, further comprising a detectable moiety attached thereto, such as a chromogenic moiety, a fluorogenic moiety, a radioactive moiety and a light-emitting moiety.

105. A set of at least two oligonucleotides each of said oligonucleotides being specifically hybridizable with a nucleic acid sequence encoding an ErbB-2 polypeptide including an amino acid being at least 71% homologous to SEQ ID NO:5, as determined using the BlastP software of the National Center of Biotechnology Information (NCBI) using default parameters, at least one pair of said oligonucleotides being designed for directing an exponential amplification of at least 15 contigous nucleotides of said nucleic acid sequence in an exponential amplification reaction such as a polymerase chain reaction (PCR).

106. A kit comprising packaging material, a vials stand being packaged by said packaging material and at least one vial in said vials stand in which said set of at least two oligonucleotides of claim 105 is contained.

107. An exponential amplification reaction product obtained by reacting in an exponential amplification reaction a pair of oligonucleotides each of said oligonucleotides being specifically hybridizable with a nucleic acid sequence encoding an ErbB-2 polypeptide including an amino acid being at least 71% homologous to SEQ ID NO:5, as determined using the BlastP software of the National Center of Biotechnology Information (NCBI) using default parameters, with said nucleid acid sequence in an exponential amplification reaction, said pair of oligonucleotides being designed for directing exponential amplification of at least 15 contigous nucleotides of said nucleic acid sequence.

108. A pharmaceutical composition comprising a therapeutically effective amount of at least an active portion of an ErbB-2 polypeptide including an amino acid sequence being at least 71% homologous to SEQ ID NO:5, as determined using the BlastP software of the National Center of Biotechnology Information (NCBI) using default parameters.

109. A pharmaceutical composition comprising a therapeutically effective amount of ErbB-2 variant polypeptide of claim 24.

110. The pharmaceutical composition of claim 109, wherein said polypeptide is as set forth in SEQ ID NO:2, 5 or 14.

111. The pharmaceutical composition of claim 109, wherein said active portion of said polypeptide is as set forth in SEQ ID NO:5.

112. The pharmaceutical composition of claim 111, wherein said active portion of said polypeptide is encoded by nucleotide coordinates 2097-2320 of SEQ ID NO: 1.

113. A pharmaceutical composition comprising a therapeutically effective amount of at least an active portion of an ErbB-2 polypeptide including an amino acid sequence being at least 69% homologous to SEQ ID NO:6, as determined using the BlastP software of the National Center of Biotechnology Information (NCBI) using default parameters.

114. The pharmaceutical composition of claim 113, wherein said polypeptide is as set forth in SEQ ID NO:4, 6 or 16.

115. The pharmaceutical composition of claim 113, wherein said active portion of said polypeptide is as set forth in SEQ ID NO:6.

116. The pharmaceutical composition of claim 115, wherein said active portion of said polypeptide is encoded by nucleotide coordinates 1664-1944 of SEQ ID NO:3.

117. A method of diagnosing predisposition to, or presence, or prognosis, or monitoring the progression of, or a responsiveness to treatment, of ErbB-related cancer in a subject, the method comprising determining a level of a polypeptide at least 71% homologous to SEQ ID NO:5, as determined using the Blastn software of the National Center of Biotechnology information (NCBI) using default parameters or of a polynucleotide encoding said polypeptide in a biological sample obtained from the subject, wherein said level of said polynucleotide or said level of said polypeptide level is correlatable with predisposition to, or presence or absence of the cancer, thereby diagnosing predisposition to, or presence of ErbB-related cancer in the subject.

118. A method of diagnosing predisposition to, or presence, or prognosis, or monitoring the progression of, or a responsiveness to treatment of, ErbB-2 related cancer in a subject, the method comprising determining a level of a polypeptide of claim 24, or of a polynucleotide encoding said polypeptide in a biological sample obtained from the subject, wherein said level of said polynucleotide or said level of said polypeptide level is correlatable with predisposition to, or presence or absence of the cancer, thereby diagnosing predisposition to, or presence of ErbB-related cancer in the subject.

119. The method of claim 117, wherein said ErbB-related cancer comprises one or both of ovarian cancer or breast cancer.

120. The method of claim 117, wherein said determining level of said polypeptide is effected is effected via an assay selected from the group consisting of immunohistochemistry, ELISA, RIA, Western blot analysis, FACS analysis, an immunofluorescence assay, and a light emission immunoassay.

121. The method of claim 117, wherein said determining level of said polynucleotide is effected via an assay selected from the group consisting of PCR, RT-PCR, chip hybridization, RNase protection, in-situ hybridization, primer extension, Southern blot, Northern blot and dot blot analysis.

122. The method of claim 117, wherein said polynucleotide is selected from the group consisting of SEQ ID NOs: 1, 7 and 13.

123. The method of claim 117, wherein said polypeptide is selected from the group consisting of SEQ ID NOs: 2, 5 and 14.

124. A method of diagnosing predisposition to, or presence of ErbB-related cancer in a subject, the method comprising determining a level of a polypeptide at least 69% homologous to SEQ ID NO:6, as determined using the Blastn software of the National Center of Biotechnology information (NCBI) using default parameters or of a polynucleotide encoding said polypeptide in a biological sample obtained from the subject, wherein said level of said polynucleotide or said level of said polypeptide level is correlatable with predisposition to, or presence or absence of the cancer, thereby diagnosing predisposition to, or presence of ErbB-related cancer in the subject.

125. The method of claim 124, wherein said determining level of said polypeptide is effected is effected via an assay selected from the group consisting of immunohistochemistry, ELISA, RIA, Western blot analysis, FACS analysis, an immunofluorescence assay, and a light emission immunoassay.

126. The method of claim 124, wherein said determining level of said polynucleotide is effected via an assay selected from the group consisting of PCR, RT-PCR, chip hybridization, RNase protection, in-situ hybridization, primer extension, Southern blot, Northern blot and dot blot analysis.

127. The method of claim 124, wherein said polynucleotide is selected from the group consisting of SEQ ID NOs: 3, 8 and 15.

128. The method of claim 124, wherein said polypeptide is selected from the group consisting of SEQ ID NOs: 4, 6 and 16.

129. A method of treating ErbB-related cancer in a subject, the method comprising specifically upregulating in the subject expression of an ErbB-2 polypeptide including an amino acid sequence being at least 71% homologous to SEQ ID NO:5 as determined using the Blastp software of the National Center of Biotechnology information (NCBI) using default parameters.

130. The method of claim 129, wherein said upregulating expression of said polypeptide is effected by:
(i) administering said polypeptide to the subject; and/or
(ii) administering an expressible polynucleotide encoding said polypeptide to the subject.

131. A method of treating ErbB-related cancer in a subject, the method comprising specifically upregulating in the subject expression of an ErbB-2 polypeptide including an amino acid sequence being at least 69% homologous to SEQ ID NO:6, as determined using the Blastp software of the National Center of Biotechnology information (NCBI) using default parameters.

132. The method of claim 131, wherein said upregulating expression of said polypeptide is effected by:
(i) administering said polypeptide to the subject; and/or
(ii) administering an expressible polynucleotide encoding said polypeptide to the subject.

133. A method of treating ErbB-related cancer in a subject, the method comprising specifically downregulating in the subject expression of an ErbB-2 polypeptide including an amino acid sequence being at least 71% homologous to SEQ ID NO:5 as determined using the Blastp software of the National Center of Biotechnology information (NCBI) using default parameters.

134. The method of claim 133, wherein specifically down-regulating expression of said polypeptide is effected by providing to the subject an antibody directed at an amino acid sequence set forth in SEQ ID NO:5.

135. The method of claim 133, wherein specifically down-regulating expression of said polypeptide is effected by providing to the subject an oligonucleotide capable of specifically inactivating said polynucleotide.

136. The method of claim 135, wherein said oligonucleotide is directed at a nucleic acid sequence set forth in SEQ ID NO:7.

137. A method of treating ErbB-related cancer in a subject, the method comprising specifically downregulating in the subject expression of an ErbB-2 polypeptide including an amino acid sequence being at least 69% homologous to SEQ ID NO:6 as determined using the Blastp software of the National Center of Biotechnology information (NCBI) using default parameters.

138. The method of claim 137, wherein specifically down-regulating expression of said polypeptide is effected by providing to the subject an antibody directed at an amino acid sequence set forth in SEQ ID NO:6.

139. The method of claim 137, wherein specifically down-regulating expression of said polypeptide is effected by providing to the subject an oligonucleotide capable of specifically inactivating said polynucleotide.

140. The method of claim 139, wherein said oligonucleotide is directed at a nucleic acid sequence set forth in SEQ ID NO:8.

141. A kit for diagnosing ErbB-related cancer or a predisposition thereto in a subject, the kit comprising the antibody or antibody fragment of claim 24 and reagents for detecting hybridization of the antibody or antibody fragment.

142. The kit of claim 141, wherein detecting hybridization of the antibody or antibody fragment is effected by an assay selected from the group consisting of immunohistochemistry, ELISA, RIA, Western blot analysis, FACS analysis, an immunofluorescence assay, and a light emission immunoassay.

143. The kit of claim 141, wherein said antibody or antibody fragment is coupled to an enzyme.

144. The kit of claim 141, wherein said antibody or antibody fragment is coupled to a detectable moiety selected from the group consisting of a chromogenic moiety, a fluorogenic moiety, a radioactive moiety and a light-emitting moiety.

145. A kit for diagnosing ErbB-related cancer or a predisposition thereto in a subject, the kit comprising the antibody or antibody fragment of claim 29 and reagents for detecting hybridization of the antibody or antibody fragment.

146. The kit of claim 145, wherein detecting hybridization of the antibody or antibody fragment is effected by an assay selected from the group consisting of immunohistochemistry, ELISA, RIA, Western blot analysis, FACS analysis, an immunofluorescence assay, and a light emission immunoassay.

147. The kit of claim 145, wherein said antibody or antibody fragment is coupled to an enzyme.

148. The kit of claim 145, wherein said antibody or antibody fragment is coupled to a detectable moiety selected from the group consisting of a chromogenic moiety, a fluorogenic moiety, a radioactive moiety and a light-emitting moiety.

149. A kit for diagnosing ErbB-related cancer or a predisposition thereto in a subject, the kit comprising the oligonucleotide of claim 91 and at least one reagent for detecting hybridization of the oligonucleotide.

150. The kit of claim 149, wherein said at least one reagent is selected suitable for detecting hybridization via an assay selected from the group consisting of PCR, RT-PCR, chip hybridization, RNase protection, in-situ hybridization, primer extension, Southern blot, Northern blot and dot blot analysis.

151. A kit for diagnosing ErbB-related cancer or a predisposition thereto in a subject, the kit comprising the oligonucleotide of claim 96 and at least one reagent for detecting hybridization of the oligonucleotide.

152. The kit of claim 151, wherein said at least one reagent is selected suitable for detecting hybridization via an assay selected from the group consisting of PCR, RT-PCR, chip hybridization, RNase protection, in-situ hybridization, primer extension, Southern blot, Northern blot and dot blot analysis.

153. An isolated polynucleotide comprising a nucleic acid sequence encoding at least an active portion of an ErbB-2 polypeptide including an amino acid sequence being at least 89% homologous to SEQ ID NO: 11, as determined using the BlastP software of the National Center of Biotechnology Information (NCBI) using default parameters.

154. The isolated polynucleotide of claim 153, wherein said ErbB-2 polypeptide is as set forth in SEQ ID NO:10, 11, 14 or 16.

155. The isolated polynucleotide of claim 153, wherein said nucleic acid sequence is as set forth in SEQ ID NO. 9, 12, 13 or 15.

156. The isolated polynucleotide of claim 153, wherein said active portion of said polypeptide is as set forth in SEQ ID NO:11.

157. The isolated polynucleotide of claim 156, wherein said active portion of said polypeptide is encoded by nucleotide coordinates 1299-1415 of SEQ ID NO:9.

158. An isolated polypeptide comprising an ErbB-2 polypeptide including an amino acid sequence being at least 89% homologous to SEQ ID NO: 11, as determined using the BlastP software of the National Center of Biotechnology information (NCBI) using default parameters or an active portion thereof.

159. The isolated polypeptide of claim 158, wherein said amino acid sequence is as set forth in SEQ ID NO:10, 11, 14 or 16.

160. The isolated polypeptide of claim 158, wherein said active portion of the polypeptide is as set forth is SEQ ID NO:11.

161. An isolated polypeptide comprising an amino acid sequence as set forth in SEQ ID NO:1, 11, 14 or 16.

162. An antibody or an antibody fragment being capable of specifically binding an ErbB-2 polypeptide including an amino acid sequence being at least 89% homologous to SEQ ID NO:11, as determined using the BlastP software of the National Center of Biotechnology Information (NCBI) using default parameters.

163. An oligonucleotide specifically hybridizable with a nucleic acid sequence encoding an ErbB-2 polypeptide including an amino acid being at least 89% homologous to SEQ ID NO:12, as determined using the BlastP software of the National Center of Biotechnology Information (NCBI) using default parameters.

164. An isolated polynucleotide comprising a nucleic acid sequence encoding an ErbB-2 polypeptide including an amino acid sequence at least 70% homologous to SEQ ID NO:28, as determined using the protein Blast search software for short, nearly exact matches of the National Center of Biotechnology Information (NCBI) using default parameters.

165. The isolated polynucleotide of claim 164, wherein said ErbB-2 polypeptide is as set forth in SEQ ID NO:26.

166. The isolated polynucleotide of claim 164, wherein said nucleic acid sequence is as set forth in SEQ ID NO:25.

167. An isolated polynucleotide as set forth in SEQ ID NO:25 or an active portion thereof.

168. An isolated polynucleotide encoding at least an active portion of an ErbB-2 polypeptide including an inositol phosphate binding domain.

169. The isolated polynucleotide of claim 168, wherein said ErbB-2 polypeptide is as set forth in SEQ ID NO:26.

170. The isolated polynucleotide of claim 168, wherein said active portion is encoded by nucleic acid sequence coordinates 1171-1314 of SEQ ID NO:25.

171. The isolated polynucleotide of claim 168, wherein said inositol phosphate binding domain is encoded by nucleic acid sequence coordinates 1171-1314 of SEQ ID NO:25.

172. The isolated polynucleotide of claim 168, wherein said inositol phosphate binding domain is a pleckstrin homology domain.

173. An isolated polypeptide comprising an ErbB-2 polypeptide including an amino acid sequence being at least 70% homologous to SEQ ID NO:28, as determined using the protein Blast search software for short, nearly exact matches of the National Center of Biotechnology Information (NCBI) using default parameters.

174. The isolated polypeptide of claim 173, wherein said ErbB-2 polypeptide is as set forth in SEQ ID NO:26.

175. A nucleic acid construct comprising the isolated polypeptide of claim 164.

176. A host cell comprising the nucleic acid construct of claim 175.

177. An antibody or an antibody fragment being capable of specifically binding a polypeptide sequence at least 70% homologous to SEQ ID NO:28, as determined using the protein Blast search software for short, nearly exact matches of the National Center of Biotechnology Information (NCBI) using default parameters.

178. The antibody or antibody fragment of claim 177, wherein said polypeptide is as set forth in SEQ ID NO:26.

179. An oligonucleotide specifically hybridizable with a nucleic acid sequence encoding a polypeptide at least 70% homologous to SEQ ID NO:28, as determined using the protein Blast search software for short, nearly exact matches of the National Center of Biotechnology Information (NCBI) using default parameters.

180. The oligonucleotide of claim 179 is as set forth in SEQ ID NO:31 or 32.

181. The oligonucleotide of claim 179, wherein said oligonucleotide is a single or double stranded.

182. The oligonucleotide of claim 179, wherein said oligonucleotide is at least 10 bases long.

183. The oligonucleotide of claim 179, wherein said oligonucleotide is hybridizable in either sense or antisense orientation.

184. A kit for diagnosing ErbB-related cancer or a predisposition thereto in a subject, the kit comprising the oligonucleotide of claim 182 and at least one reagent for detecting hybridization of the oligonucleotide.

185. The kit of claim 184, wherein said at least one reagent is selected suitable for detecting hybridization via an assay selected from the group consisting of PCR, RT-PCR, chip hybridization, RNase protection, in-situ hybridization, primer extension, Southern blot, Northern blot and dot blot analysis.

186. A kit for diagnosing ErbB-related cancer or a predisposition thereto in a subject, the kit comprising the antibody or antibody fragment of claim 180 and reagents for detecting hybridization of the antibody or antibody fragment.

187. The kit of claim 186, wherein detecting hybridization of the antibody or antibody fragment is effected by an assay selected from the group consisting of immunohistochemistry, ELISA, RIA, Western blot analysis, FACS analysis, an immunofluorescence assay, and a light emission immunoassay.

188. The kit of claim 186, wherein said antibody or antibody fragment is coupled to an enzyme.

189. The kit of claim 186, wherein said antibody or antibody fragment is coupled to a detectable moiety selected from the group consisting of a chromogenic moiety, a fluorogenic moiety, a radioactive moiety and a light-emitting moiety.

190. A method of diagnosing predisposition to, or presence of ErbB-related cancer in a subject, the method comprising determining a level of a polypeptide at least 70% homologous to SEQ ID NO:28, as determined using the protein Blast search software for short, nearly exact matches of the National Center of Biotechnology Information (NCBI) using default parameters or of a polynucleotide encoding said polypeptide in a biological sample obtained from the subject, wherein said level of said polynucleotide or said level of said polypeptide level is correlatable with predisposition to, or presence or absence of the cancer, thereby diagnosing predisposition to, or presence of ErbB-related cancer in the subject.

191. The method of claim 190, wherein said determining level of said polypeptide is effected is effected via an assay selected from the group consisting of immunohistochemistry, ELISA, RIA, Western blot analysis, FACS analysis, an immunofluorescence assay, and a light emission immunoassay.

192. The method of claim 190, wherein said determining level of said polynucleotide is effected via an assay selected from the group consisting of PCR, RT-PCR, chip hybridization, RNase protection, in-situ hybridization, primer extension, Southern blot, Northern blot and dot blot analysis.

193. The method of claim 190, wherein said polynucleotide is as set forth in SEQ ID NO:25.

194. The method of claim 190, wherein said polypeptide is as set forth in SEQ ID NO:26.

195. A method of treating ErbB-related cancer in a subject, the method comprising specifically upregulating in the subject expression of a polypeptide at least 70% homologous to SEQ ID NO:28, as determined using the protein Blast search software for short, nearly exact matches of the National Center of Biotechnology Information (NCBI) using default parameters.

196. The method of claim 195, wherein said upregulating expression of said polypeptide is effected by:
(i) administering said polypeptide to the subject; and/or
(ii) administering an expressible polynucleotide encoding said polypeptide to the subject.

197. A biomarker for detecting breast cancer, comprising ErbB-2-long variant I or IV sequence or a fragment thereof.

198. A biomarker for detecting breast cancer, comprising ErbB-2-short variant II or V sequence or a fragment thereof.

199. The biomarker of claim 197, wherein said fragment comprises a tail of ErbB-2-long variant, comprising a polypeptide having the sequence RLAWTPGCTLHCPSLPHWMLGGHCCREGTP (SEQ ID NO: 5) or a polynucleotide encoding said polypeptide.

200. The biomarker of claim 197, wherein said fragment comprises a tail of ErbB-2-short variant, comprising a polypeptide having the sequence GKTGSPVCALPICQHTAVPRGPWQQRSWTCADCPSLCTLLDSAQLWLAWPL GMASLAGSYLPWHPSLPLCF (SEQ ID NO: 6) or a polynucleotide encoding said polypeptide.

201. A primer pair for use in detecting the biomarker of claim 197, comprising a primer pair capable of amplifying ErbB-2 variants or a fragment thereof.

202. The primer pair of claim 201, comprising ErbB-2-long variant forward primer: TGTGAGGGACACAGGCAAAGT (SEQ ID NO: 48); and ErbB-2-long variant—Reverse primer: CCCACCATCCCCAGTTAAGAA (SEQ ID NO: 49).

203. The primer pair of claim 201, comprising ErbB-2-short variant—forward primer: CAGCGTTCTTGGACTTGTGC (SEQ ID NO: 51); and ErbB-2-short variant—Reverse primer: CCAGCTAGAGAAGCCATGCC (SEQ ID NO: 52).

204. An amplicon obtained through the use of a primer pair according to claim 201.

205. The amplicon of claim 204, comprising ErbB-2-long variant amplicon:
(SEQ ID NO 50)
TGTGAGGGACACAGGCAAAGTTCAATTCCTTGGAAGTCAAGGGAGACTGA
GAAGAGTACAGCTGCAGCACTGAGGGAGTGATGAATTCTTAACTGGGGAT
GGTGGG.

206. The amplicon of claim 204, comprising ErbB-2-short variant amplicon:
(SEQ ID NO 53)
CAGCGTTCTTGGACTTGTGCAGACTGCCCGTCTCTGTGCACCCTTCTTGAC
TCAGCACAGCTCTGGCTGGCTTGGCCTCTTGGCATGGCTTCTCTAGCTGG.

207. An assay for detecting breast cancer, comprising detecting overexpression of ErbB-2-long or short variants or a fragment thereof.

208. The assay of claim 207, wherein said assay comprises a NAT-based technology.

209. A method for detecting breast cancer, comprising detecting overexpression of the ErbB-2 variants or a fragment thereof.


SEQUENCE LISTING (53 sequences)

SEQ ID NO: 1 - DNA, 2320 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccaggttggcctggaccccaggatgtacccttcattgcccttcactcccccactggatgctgggtggtcactgctgtagggaggggaccccctgacatatgtcccttcccacccactcttccactgtggaacctcctgtcattttccacttcaccaagtgacagaggacctgctcagatgctgaggggaggggactgcaaggaaagatggctaggaaacccagtccct

SEQ ID NO: 2 - protein, 678 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRARLAWTPGCTLHCPSLPHWMLGGHCCREGTP

SEQ ID NO: 3 - DNA, 1944 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtggtaagacagggagcccagtgtgcgcactccccatctgccagcacacagcagtgcccagggggccctggcagcagcgttcttggacttgtgcagactgcccgtctctgtgcacccttcttgactcagcacagctctggctggcttggcctcttggcatggcttctctagctgggtcctacctgccttggcatccttccctccccctctgtttctgaaatctcagaactcttcctctccctacatcggccccacctgtccccacccctccagcccacagccat

SEQ ID NO: 4 - protein, 575 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECGKTGSPVCALPICQHTAVPRGPWQQRSWTCADCPSLCTLLDSAQLWLAWPLGMASLAGSYLPWHPSLPLCF

SEQ ID NO: 5 - protein, 30 aa, Homo sapiens
RLAWTPGCTLHCPSLPHWMLGGHCCREGTP

SEQ ID NO: 6 - protein, 71 aa, Homo sapiens
GKTGSPVCALPICQHTAVPRGPWQQRSWTCADCPSLCTLLDSAQLWLAWPLGMASLAGSYLPWHPSLPLCF

SEQ ID NO: 7 - DNA, 224 bp, Homo sapiens
gttggcctggaccccaggatgtacccttcattgcccttcactcccccactggatgctgggtggtcactgctgtagggaggggaccccctgacatatgtcccttcccacccactcttccactgtggaacctcctgtcattttccacttcaccaagtgacagaggacctgctcagatgctgaggggaggggactgcaaggaaagatggctaggaaacccagtccct

SEQ ID NO: 8 - DNA, 281 bp, Homo sapiens
gtaagacagggagcccagtgtgcgcactccccatctgccagcacacagcagtgcccagggggccctggcagcagcgttcttggacttgtgcagactgcccgtctctgtgcacccttcttgactcagcacagctctggctggcttggcctcttggcatggcttctctagctgggtcctacctgccttggcatccttccctccccctctgtttctgaaatctcagaactcttcctctccctacatcggccccacctgtccccacccctccagcccacagccat

SEQ ID NO: 9 - DNA, 4647 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggagtctcactctgtcagcaggctggagtgcagtggtacgatcttggctcactgcaacctctgcctcctggattcaagcaattctcctgcctcagtctcctgagtagctgggactacagggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 10 - protein, 1294 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGVSLCQQAGVQWYDLGSLQPLPPGFKQFSCLSLLSSWDYRDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 11 - protein, 39 aa, Homo sapiens
VSLCQQAGVQWYDLGSLQPLPPGFKQFSCLSLLSSWDYR

SEQ ID NO: 12 - DNA, 117 bp, Homo sapiens
agtctcactctgtcagcaggctggagtgcagtggtacgatcttggctcactgcaacctctgcctcctggattcaagcaattctcctgcctcagtctcctgagtagctgggactacag

SEQ ID NO: 13 - DNA, 2437 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggagtctcactctgtcagcaggctggagtgcagtggtacgatcttggctcactgcaacctctgcctcctggattcaagcaattctcctgcctcagtctcctgagtagctgggactacagggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccaggttggcctggaccccaggatgtacccttcattgcccttcactcccccactggatgctgggtggtcactgctgtagggaggggaccccctgacatatgtcccttcccacccactcttccactgtggaacctcctgtcattttccacttcaccaagtgacagaggacctgctcagatgctgaggggaggggactgcaaggaaagatggctaggaaacccagtccct

SEQ ID NO: 14 - protein, 717 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGVSLCQQAGVQWYDLGSLQPLPPGFKQFSCLSLLSSWDYRDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRARLAWTPGCTLHCPSLPHWMLGGHCCREGTP

SEQ ID NO: 15 - DNA, 2061 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggagtctcactctgtcagcaggctggagtgcagtggtacgatcttggctcactgcaacctctgcctcctggattcaagcaattctcctgcctcagtctcctgagtagctgggactacagggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtggtaagacagggagcccagtgtgcgcactccccatctgccagcacacagcagtgcccagggggccctggcagcagcgttcttggacttgtgcagactgcccgtctctgtgcacccttcttgactcagcacagctctggctggcttggcctcttggcatggcttctctagctgggtcctacctgccttggcatccttccctccccctctgtttctgaaatctcagaactcttcctctccctacatcggccccacctgtccccacccctccagcccacagccat

SEQ ID NO: 16 - protein, 614 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGVSLCQQAGVQWYDLGSLQPLPPGFKQFSCLSLLSSWDYRDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECGKTGSPVCALPICQHTAVPRGPWQQRSWTCADCPSLCTLLDSAQLWLAWPLGMASLAGSYLPWHPSLPLCF

SEQ ID NO: 17 - DNA, 22 bp, Artificial sequence
  Single strand DNA oligonucleotide
gtctctgcggtggttggcattc

SEQ ID NO: 18 - DNA, 22 bp, Artificial sequence
  Single strand DNA oligonucleotide
gagcggccaagtccctgtgtac

SEQ ID NO: 19 - DNA, 24 bp, Artificial sequence
  Single strand DNA oligonucleotide
agaatggctcagtgacctgttttg

SEQ ID NO: 20 - DNA, 24 bp, Artificial sequence
  Single strand DNA oligonucleotide
ctcttctcagtctcccttgacttc

SEQ ID NO: 21 - DNA, 24 bp, Artificial sequence
  Single strand DNA oligonucleotide
catttctgccggagagctttgatg

SEQ ID NO: 22 - DNA, 23 bp, Artificial sequence
  Single strand DNA oligonucleotide
caggtaggacccagctagagaag

SEQ ID NO: 23 - DNA, 25 bp, Artificial sequence
  Single strand DNA oligonucleotide
cagtgattataggctttcgctctaa

SEQ ID NO: 24 - DNA, 23 bp, Artificial sequence
  Single strand DNA oligonucleotide
cagggctattggttgaatgagta

SEQ ID NO: 25 - DNA, 1320 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagggacccagcctccaacactgccccgctccagccagagcagctccaagtgtttgagactctggaagagatcacaggttacctatacatctcagcatggccggacagcctgcctgacctcagcgtcttccagaacctgcaagtaatccggg

SEQ ID NO: 26 - protein, 387 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARGTQPPTLPRSSQSSSKCLRLWKRSQVTYTSQHGRTACLTSASSRTCK

SEQ ID NO: 27 - DNA, 144 bp, Homo sapiens
  misc_feature: Unique nucleotide sequence of variant 6
gggacccagcctccaacactgccccgctccagccagagcagctccaagtgtttgagactctggaagagatcacaggttacctatacatctcagcatggccggacagcctgcctgacctcagcgtcttccagaacctgcaagtaa

SEQ ID NO: 28 - protein, 47 aa, Homo sapiens
  misc_feature: Unique amino acid sequence of variant 6
GTQPPTLPRSSQSSSKCLRLWKRSQVTYTSQHGRTACLTSASSRTCK

SEQ ID NO: 29 - DNA, 20 bp, Artificial sequence
  Single strand DNA oligonucleotide
atggagctggcggccttgtg

SEQ ID NO: 30 - DNA, 26 bp, Artificial sequence
  Single strand DNA oligonucleotide
cacagtggaagagtgggtgggaaggg

SEQ ID NO: 31 - DNA, 26 bp, Artificial sequence
  Single strand DNA oligonucleotide
aagccctgtgcccgagggacccagcc

SEQ ID NO: 32 - DNA, 24 bp, Artificial sequence
  Single strand DNA oligonucleotide
gttggaggctgggtccctcgggca

SEQ ID NO: 33 - DNA, 21 bp, Artificial sequence
  Single strand DNA oligonucleotide
tgacactggcaaaacaatgca

SEQ ID NO: 34 - DNA, 21 bp, Artificial sequence
  Single strand DNA oligonucleotide
ggtccttttcaccagcaagct

SEQ ID NO: 35 - DNA, 94 bp, Artificial sequence
  HPRT1 real time PCR amplicon
tgacactggcaaaacaatgcagactttgctttccttggtcaggcagtataatccaaagatggtcaaggtcgcaagcttgctggtgaaaaggacc

SEQ ID NO: 36 - DNA, 19 bp, Artificial sequence
  Single strand DNA oligonucleotide
tgagagtgattcgcgtggg

SEQ ID NO: 37 - DNA, 21 bp, Artificial sequence
  Single strand DNA oligonucleotide
ccagggtacgaggctttcaat

SEQ ID NO: 38 - DNA, 91 bp, Artificial sequence
  PBGD real time PCR amplicon
tgagagtgattcgcgtgggtacccgcaagagccagcttgctcgcatacagacggacagtgtggtggcaacattgaaagcctcgtaccctgg

SEQ ID NO: 39 - DNA, 20 bp, Artificial sequence
  Single strand DNA oligonucleotide
tgggaacaagagggcatctg

SEQ ID NO: 40 - DNA, 22 bp, Artificial sequence
  Single strand DNA oligonucleotide
ccaccactgcatcaaattcatg

SEQ ID NO: 41 - DNA, 86 bp, Artificial sequence
  SDHA real time PCR amplicon
tgggaacaagagggcatctgctaaagtttcagattccatttctgctcagtatccagtagtggatcatgaatttgatgcagtggtgg

SEQ ID NO: 42 - DNA, 20 bp, Artificial sequence
  Single strand DNA oligonucleotide
gaggccgtcaccaagaacat

SEQ ID NO: 43 - DNA, 19 bp, Artificial sequence
  Single strand DNA oligonucleotide
ggacagccggtcagagctc

SEQ ID NO: 44 - DNA, 111 bp, Artificial sequence
  G6PD real time PCR amplicon
gaggccgtcaccaagaacattcacgagtcctgcatgagccagataggctggaaccgcatcatcgtggagaagcccttcgggagggacctgcagagctctgaccggctgtcc

SEQ ID NO: 45 - DNA, 19 bp, Artificial sequence
  Single strand DNA oligonucleotide
cccatatgtctcccgcctt

SEQ ID NO: 46 - DNA, 21 bp, Artificial sequence
  Single strand DNA oligonucleotide
acatggtctaagaggcagcca

SEQ ID NO: 47 - DNA, 93 bp, Artificial sequence
  ERBB-2-WT real time PCR amplicon
cccatatgtctcccgccttctgggcatctgcctgacatccacggtgcagctggtgacacagcttatgccctatggctgcctcttagaccatgt

SEQ ID NO: 48 - DNA, 21 bp, Artificial sequence
  Single strand DNA oligonucleotide
tgtgagggacacaggcaaagt

SEQ ID NO: 49 - DNA, 21 bp, Artificial sequence
  Single strand DNA oligonucleotide
cccaccatccccagttaagaa

SEQ ID NO: 50 - DNA, 106 bp, Artificial sequence
  ERBB-2-long real time PCR amplicon
tgtgagggacacaggcaaagttcaattccttggaagtcaagggagactgagaagagtacagctgcagcactgagggagtgatgaattcttaactggggatggtggg

SEQ ID NO: 51 - DNA, 20 bp, Artificial sequence
  Single strand DNA oligonucleotide
cagcgttcttggacttgtgc

SEQ ID NO: 52 - DNA, 20 bp, Artificial sequence
  Single strand DNA oligonucleotide
ccagctagagaagccatgcc

SEQ ID NO: 53 - DNA, 101 bp, Artificial sequence
  ERBB-2-short real time PCR amplicon
cagcgttcttggacttgtgcagactgcccgtctctgtgcacccttcttgactcagcacagctctggctggcttggcctcttggcatggcttctctagctgg